SEQUENCE LISTING (966 sequences)

SEQ ID NO: 1 - protein, 17 aa, Artificial sequence
  Synthetic peptide
NSNNLDSKVGGNYNYLY

SEQ ID NO: 2 - protein, 30 aa, Artificial sequence
  Synthetic peptide
IYSKHTPINLVRDLPQGFSALEPLVDLPIG

SEQ ID NO: 3 - protein, 31 aa, Artificial sequence
  Synthetic peptide
LPFFSNVTWFHAIHVSGTNGTKRFDNPVLPF

SEQ ID NO: 4 - protein, 25 aa, Artificial sequence
  Synthetic peptide
TPCNGVEGFNCYFPLQSYGFQPTNG

SEQ ID NO: 5 - protein, 30 aa, Artificial sequence
  Synthetic peptide
FQTLLALHRSYLTPGDSSSGWTAGAAAYYV

SEQ ID NO: 6 - protein, 15 aa, Artificial sequence
  Synthetic peptide
FEYVSQPFLMDLEGK

SEQ ID NO: 7 - protein, 15 aa, Artificial sequence
  Synthetic peptide
FNATRFASVYAWNRK

SEQ ID NO: 8 - protein, 15 aa, Artificial sequence
  Synthetic peptide
LPFFSNVTWFHAIHV

SEQ ID NO: 9 - protein, 15 aa, Artificial sequence
  Synthetic peptide
PFFSNVTWFHAIHVS

SEQ ID NO: 10 - protein, 15 aa, Artificial sequence
  Synthetic peptide
NGVEGFNCYFPLQSY

SEQ ID NO: 11 - protein, 15 aa, Artificial sequence
  Synthetic peptide
GVEGFNCYFPLQSYG

SEQ ID NO: 12 - protein, 9 aa, Artificial sequence
  Synthetic peptide
SPRRARSVA

SEQ ID NO: 13 - protein, 9 aa, Artificial sequence
  Synthetic peptide
SKVGGNYNY

SEQ ID NO: 14 - protein, 9 aa, Artificial sequence
  Synthetic peptide
HVSGTNGTK

SEQ ID NO: 15 - protein, 9 aa, Artificial sequence
  Synthetic peptide
VTWFHAIHV

SEQ ID NO: 16 - protein, 9 aa, Artificial sequence
  Synthetic peptide
YSKHTPINL

SEQ ID NO: 17 - protein, 9 aa, Artificial sequence
  Synthetic peptide
WTAGAAAYY

SEQ ID NO: 18 - protein, 130 aa, Artificial sequence
  Synthetic peptide
MAKLSTDELLDAFKEMTLLELSDFVKKFEETFEVTAAAPVAVAAAGAAPAGAAVEAAEEQSEFDVILEAAGDKKIGVIKVVREIVSGLGLKEAKDLVDGAPKPLLEKVAKEAADEAKAKLEAAGATVTVK

SEQ ID NO: 19 - protein, 5 aa, Artificial sequence
  Synthetic peptide
EAAAK

SEQ ID NO: 20 - protein, 5 aa, Artificial sequence
  Synthetic peptide
GPGPG

SEQ ID NO: 21 - protein, 331 aa, Artificial sequence
  Synthetic peptide
MAKLSTDELLDAFKEMTLLELSDFVKKFEETFEVTAAAPVAVAAAGAAPAGAAVEAAEEQSEFDVILEAAGDKKIGVIKVVREIVSGLGLKEAKDLVDGAPKPLLEKVAKEAADEAKAKLEAAGATVTVKEAAAKFEYVSQPFLMDLEGKGPGPGFNATRFASVYAWNRKAAYSPRRARSVAKKNSNNLDSKVGGNYNYLYKKIYSKHTPINLVRDLPQGFSALEPLVDLPIGKKLPFFSNVTWFHAIHVSGTNGTKRFDNPVLPFKKTPCNGVEGFNCYFPLQSYGFQPTNGKKFQTLLALHRSYLTPGDSSSGWTAGAAAYYVHHHHHH

SEQ ID NO: 22 - protein, 325 aa, Artificial sequence
  Synthetic peptide
MAKLSTDELLDAFKEMTLLELSDFVKKFEETFEVTAAAPVAVAAAGAAPAGAAVEAAEEQSEFDVILEAAGDKKIGVIKVVREIVSGLGLKEAKDLVDGAPKPLLEKVAKEAADEAKAKLEAAGATVTVKEAAAKFEYVSQPFLMDLEGKGPGPGFNATRFASVYAWNRKAAYSPRRARSVAKKNSNNLDSKVGGNYNYLYKKIYSKHTPINLVRDLPQGFSALEPLVDLPIGKKLPFFSNVTWFHAIHVSGTNGTKRFDNPVLPFKKTPCNGVEGFNCYFPLQSYGFQPTNGKKFQTLLALHRSYLTPGDSSSGWTAGAAAYYV

SEQ ID NO: 23 - protein, 190 aa, Artificial sequence
  Synthetic peptide
FEYVSQPFLMDLEGKGPGPGFNATRFASVYAWNRKAAYSPRRARSVAKKNSNNLDSKVGGNYNYLYKKIYSKHTPINLVRDLPQGFSALEPLVDLPIGKKLPFFSNVTWFHAIHVSGTNGTKRFDNPVLPFKKTPCNGVEGFNCYFPLQSYGFQPTNGKKFQTLLALHRSYLTPGDSSSGWTAGAAAYYV

SEQ ID NO: 24 - protein, 12 aa, Artificial sequence
  Synthetic peptide
AALQIPFAMQMA

SEQ ID NO: 25 - protein, 16 aa, Artificial sequence
  Synthetic peptide
AALQIPFAMQMAYRFN

SEQ ID NO: 26 - protein, 45 aa, Artificial sequence
  Synthetic peptide
AALQIPFAMQMAYRFNGIGVTQNVLYENQKLIANQFNSAIGKIQD

SEQ ID NO: 27 - protein, 16 aa, Artificial sequence
  Synthetic peptide
ADQLTPTWRVYSTGSN

SEQ ID NO: 28 - protein, 17 aa, Artificial sequence
  Synthetic peptide
ADQLTPTWRVYSTGSNV

SEQ ID NO: 29 - protein, 18 aa, Artificial sequence
  Synthetic peptide
ADQLTPTWRVYSTGSNVF

SEQ ID NO: 30 - protein, 10 aa, Artificial sequence
  Synthetic peptide
ADSFVIRGDE

SEQ ID NO: 31 - protein, 28 aa, Artificial sequence
  Synthetic peptide
ADSFVIRGDEVRQIAPGQTGKIADYNYK

SEQ ID NO: 32 - protein, 29 aa, Artificial sequence
  Synthetic peptide
ADSFVIRGDEVRQIAPGQTGKIADYNYKL

SEQ ID NO: 33 - protein, 30 aa, Artificial sequence
  Synthetic peptide
ADSFVIRGDEVRQIAPGQTGKIADYNYKLP

SEQ ID NO: 34 - protein, 32 aa, Artificial sequence
  Synthetic peptide
ADSFVIRGDEVRQIAPGQTGKIADYNYKLPDD

SEQ ID NO: 35 - protein, 34 aa, Artificial sequence
  Synthetic peptide
ADSFVIRGDEVRQIAPGQTGKIADYNYKLPDDFT

SEQ ID NO: 36 - protein, 117 aa, Artificial sequence
  Synthetic peptide
ADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVL

SEQ ID NO: 37 - protein, 118 aa, Artificial sequence
  Synthetic peptide
ADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLS

SEQ ID NO: 38 - protein, 19 aa, Artificial sequence
  Synthetic peptide
ADYSVLYNSASFSTFKCYG

SEQ ID NO: 39 - protein, 23 aa, Artificial sequence
  Synthetic peptide
ADYSVLYNSASFSTFKCYGVSPT

SEQ ID NO: 40 - protein, 24 aa, Artificial sequence
  Synthetic peptide
ADYSVLYNSASFSTFKCYGVSPTK

SEQ ID NO: 41 - protein, 27 aa, Artificial sequence
  Synthetic peptide
ADYSVLYNSASFSTFKCYGVSPTKLND

SEQ ID NO: 42 - protein, 7 aa, Artificial sequence
  Synthetic peptide
AEHVNNS

SEQ ID NO: 43 - protein, 8 aa, Artificial sequence
  Synthetic peptide
AEHVNNSY

SEQ ID NO: 44 - protein, 9 aa, Artificial sequence
  Synthetic peptide
AEHVNNSYE

SEQ ID NO: 45 - protein, 19 aa, Artificial sequence
  Synthetic peptide
AENSVAYSNNSIAIPTNFT

SEQ ID NO: 46 - protein, 20 aa, Artificial sequence
  Synthetic peptide
AENSVAYSNNSIAIPTNFTI

SEQ ID NO: 47 - protein, 18 aa, Artificial sequence
  Synthetic peptide
AGAALQIPFAMQMAYRFN

SEQ ID NO: 48 - protein, 47 aa, Artificial sequence
  Synthetic peptide
AGAALQIPFAMQMAYRFNGIGVTQNVLYENQKLIANQFNSAIGKIQD

SEQ ID NO: 49 - protein, 52 aa, Artificial sequence
  Synthetic peptide
AGAALQIPFAMQMAYRFNGIGVTQNVLYENQKLIANQFNSAIGKIQDSLSST

SEQ ID NO: 50 - protein, 47 aa, Artificial sequence
  Synthetic peptide
AGAALQIPFAMQMAYRFNGIGVTQNVLYENQKLIANQFNSVIGKIQD

SEQ ID NO: 51 - protein, 53 aa, Artificial sequence
  Synthetic peptide
AGAALQIPFAMQMAYRFNGIGVTQNVLYENQKLIANQFNSVIGKIQDSLSSTA

SEQ ID NO: 52 - protein, 29 aa, Artificial sequence
  Synthetic peptide
AGSTPCNGVEGFNCYFPLQSYGFQPTNGV

SEQ ID NO: 53 - protein, 10 aa, Artificial sequence
  Synthetic peptide
AGTITSGWTF

SEQ ID NO: 54 - protein, 5 aa, Artificial sequence
  Synthetic peptide
AIHAD

SEQ ID NO: 55 - protein, 9 aa, Artificial sequence
  Synthetic peptide
AIHADQLTP

SEQ ID NO: 56 - protein, 14 aa, Artificial sequence
  Synthetic peptide
AIHADQLTPTWRVY

SEQ ID NO: 57 - protein, 19 aa, Artificial sequence
  Synthetic peptide
AIHADQLTPTWRVYSTGSN

SEQ ID NO: 58 - protein, 20 aa, Artificial sequence
  Synthetic peptide
AIHADQLTPTWRVYSTGSNV

SEQ ID NO: 59 - protein, 21 aa, Artificial sequence
  Synthetic peptide
AIHADQLTPTWRVYSTGSNVF

SEQ ID NO: 60 - protein, 22 aa, Artificial sequence
  Synthetic peptide
AIHADQLTPTWRVYSTGSNVFQ

SEQ ID NO: 61 - protein, 23 aa, Artificial sequence
  Synthetic peptide
AIHADQLTPTWRVYSTGSNVFQT

SEQ ID NO: 62 - protein, 37 aa, Artificial sequence
  Synthetic peptide
ALQIPFAMQMAYRFNGIGVTQNVLYENQKLIANQFNS

SEQ ID NO: 63 - protein, 5 aa, Artificial sequence
  Synthetic peptide
APATV

SEQ ID NO: 64 - protein, 18 aa, Artificial sequence
  Synthetic peptide
APATVCGPKKSTNLVKNK

SEQ ID NO: 65 - protein, 15 aa, Artificial sequence
  Synthetic peptide
ATVCGPKKSTNLVKN

SEQ ID NO: 66 - protein, 16 aa, Artificial sequence
  Synthetic peptide
ATVCGPKKSTNLVKNK

SEQ ID NO: 67 - protein, 24 aa, Artificial sequence
  Synthetic peptide
AWNSNNLDSKVGGNYNYLYRLFRK

SEQ ID NO: 68 - protein, 26 aa, Artificial sequence
  Synthetic peptide
AWNSNNLDSKVGGNYNYLYRLFRKSN

SEQ ID NO: 69 - protein, 8 aa, Artificial sequence
  Synthetic peptide
AYTNSFTR

SEQ ID NO: 70 - protein, 9 aa, Artificial sequence
  Synthetic peptide
AYTNSFTRG

SEQ ID NO: 71 - protein, 10 aa, Artificial sequence
  Synthetic peptide
AYTNSFTRGV

SEQ ID NO: 72 - protein, 11 aa, Artificial sequence
  Synthetic peptide
AYTNSFTRGVY

SEQ ID NO: 73 - protein, 12 aa, Artificial sequence
  Synthetic peptide
CGPKKSTNLVKN

SEQ ID NO: 74 - protein, 13 aa, Artificial sequence
  Synthetic peptide
CGPKKSTNLVKNK

SEQ ID NO: 75 - protein, 14 aa, Artificial sequence
  Synthetic peptide
CGPKKSTNLVKNKC

SEQ ID NO: 76 - protein, 5 aa, Artificial sequence
  Synthetic peptide
CLGDI

SEQ ID NO: 77 - protein, 7 aa, Artificial sequence
  Synthetic peptide
CLGDIAA

SEQ ID NO: 78 - protein, 23 aa, Artificial sequence
  Synthetic peptide
CNGVEGFNCYFPLQSYGFQPTNG

SEQ ID NO: 79 - protein, 29 aa, Artificial sequence
  Synthetic peptide
CVADYSVLYNSASFSTFKCYGVSPTKLND

SEQ ID NO: 80 - protein, 31 aa, Artificial sequence
  Synthetic peptide
CVIAWNSNNLDSKVGGNYNYLYRLFRKSNLK

SEQ ID NO: 81 - protein, 81 aa, Artificial sequence
  Synthetic peptide
CVIAWNSNNLDSKVGGNYNYLYRLFRKSNLKPFERDISTEIYQAGSTPCNGVEGFNCYFPLQSYGFQPTNGVGYQPYRVVV

SEQ ID NO: 82 - protein, 82 aa, Artificial sequence
  Synthetic peptide
CVIAWNSNNLDSKVGGNYNYLYRLFRKSNLKPFERDISTEIYQAGSTPCNGVEGFNCYFPLQSYGFQPTNGVGYQPYRVVVL

SEQ ID NO: 83 - protein, 86 aa, Artificial sequence
  Synthetic peptide
DFTGCVIAWNSNNLDSKVGGNYNYLYRLFRKSNLKPFERDISTEIYQAGSTPCNGVEGFNCYFPLQSYGFQPTNGVGYQPYRVVVL

SEQ ID NO: 84 - protein, 16 aa, Artificial sequence
  Synthetic peptide
DISTEIYQAGSTPCNG

SEQ ID NO: 85 - protein, 34 aa, Artificial sequence
  Synthetic peptide
DISTEIYQAGSTPCNGVEGFNCYFPLQSYGFQPT

SEQ ID NO: 86 - protein, 36 aa, Artificial sequence
  Synthetic peptide
DISTEIYQAGSTPCNGVEGFNCYFPLQSYGFQPTNG

SEQ ID NO: 87 - protein, 37 aa, Artificial sequence
  Synthetic peptide
DISTEIYQAGSTPCNGVEGFNCYFPLQSYGFQPTNGV

SEQ ID NO: 88 - protein, 43 aa, Artificial sequence
  Synthetic peptide
DISTEIYQAGSTPCNGVEGFNCYFPLQSYGFQPTNGVGYQPYR

SEQ ID NO: 89 - protein, 6 aa, Artificial sequence
  Synthetic peptide
DPQTLE

SEQ ID NO: 90 - protein, 7 aa, Artificial sequence
  Synthetic peptide
DPQTLEI

SEQ ID NO: 91 - protein, 29 aa, Artificial sequence
  Synthetic peptide
DSFVIRGDEVRQIAPGQTGKIADYNYKLP

SEQ ID NO: 92 - protein, 18 aa, Artificial sequence
  Synthetic peptide
DYSVLYNSASFSTFKCYG

SEQ ID NO: 93 - protein, 19 aa, Artificial sequence
  Synthetic peptide
EGFNCYFPLQSYGFQPTNG

SEQ ID NO: 94 - protein, 6 aa, Artificial sequence
  Synthetic peptide
EHVNNS

SEQ ID NO: 95 - protein, 7 aa, Artificial sequence
  Synthetic peptide
EHVNNSY

SEQ ID NO: 96 - protein, 8 aa, Artificial sequence
  Synthetic peptide
EHVNNSYE

SEQ ID NO: 97 - protein, 39 aa, Artificial sequence
  Synthetic peptide
EIYQAGSTPCNGVEGFNCYFPLQSYGFQPTNGVGYQPYR

SEQ ID NO: 98 - protein, 40 aa, Artificial sequence
  Synthetic peptide
EIYQAGSTPCNGVEGFNCYFPLQSYGFQPTNGVGYQPYRV

SEQ ID NO: 99 - protein, 19 aa, Artificial sequence
  Synthetic peptide
ENSVAYSNNSIAIPTNFTI

SEQ ID NO: 100 - protein, 9 aa, Artificial sequence
  Synthetic peptide
ERDISTEIY

SEQ ID NO: 101 - protein, 13 aa, Artificial sequence
  Synthetic peptide
ERDISTEIYQAGS

SEQ ID NO: 102 - protein, 18 aa, Artificial sequence
  Synthetic peptide
ERDISTEIYQAGSTPCNG

SEQ ID NO: 103 - protein, 38 aa, Artificial sequence
  Synthetic peptide
ERDISTEIYQAGSTPCNGVEGFNCYFPLQSYGFQPTNG

SEQ ID NO: 104 - protein, 39 aa, Artificial sequence
  Synthetic peptide
ERDISTEIYQAGSTPCNGVEGFNCYFPLQSYGFQPTNGV

SEQ ID NO: 105 - protein, 45 aa, Artificial sequence
  Synthetic peptide
ERDISTEIYQAGSTPCNGVEGFNCYFPLQSYGFQPTNGVGYQPYR

SEQ ID NO: 106 - protein, 47 aa, Artificial sequence
  Synthetic peptide
ERDISTEIYQAGSTPCNGVEGFNCYFPLQSYGFQPTNGVGYQPYRVV

SEQ ID NO: 107 - protein, 48 aa, Artificial sequence
  Synthetic peptide
ERDISTEIYQAGSTPCNGVEGFNCYFPLQSYGFQPTNGVGYQPYRVVV

SEQ ID NO: 108 - protein, 49 aa, Artificial sequence
  Synthetic peptide
ERDISTEIYQAGSTPCNGVEGFNCYFPLQSYGFQPTNGVGYQPYRVVVL

SEQ ID NO: 109 - protein, 52 aa, Artificial sequence
  Synthetic peptide
ERDISTEIYQAGSTPCNGVEGFNCYFPLQSYGFQPTNGVGYQPYRVVVLSFE

SEQ ID NO: 110 - protein, 9 aa, Artificial sequence
  Synthetic peptide
ESNKKFLPF

SEQ ID NO: 111 - protein, 10 aa, Artificial sequence
  Synthetic peptide
ESNKKFLPFQ

SEQ ID NO: 112 - protein, 23 aa, Artificial sequence
  Synthetic peptide
EVFNATRFASVYAWNRKRISNCV

SEQ ID NO: 113 - protein, 38 aa, Artificial sequence
  Synthetic peptide
FERDISTEIYQAGSTPCNGVEGFNCYFPLQSYGFQPTN

SEQ ID NO: 114 - protein, 39 aa, Artificial sequence
  Synthetic peptide
FERDISTEIYQAGSTPCNGVEGFNCYFPLQSYGFQPTNG

SEQ ID NO: 115 - protein, 46 aa, Artificial sequence
  Synthetic peptide
FERDISTEIYQAGSTPCNGVEGFNCYFPLQSYGFQPTNGVGYQPYR

SEQ ID NO: 116 - protein, 48 aa, Artificial sequence
  Synthetic peptide
FERDISTEIYQAGSTPCNGVEGFNCYFPLQSYGFQPTNGVGYQPYRVV

SEQ ID NO: 117 - protein, 50 aa, Artificial sequence
  Synthetic peptide
FERDISTEIYQAGSTPCNGVEGFNCYFPLQSYGFQPTNGVGYQPYRVVVL

SEQ ID NO: 118 - protein, 52 aa, Artificial sequence
  Synthetic peptide
FERDISTEIYQAGSTPCNGVEGFNCYFPLQSYGFQPTNGVGYQPYRVVVLSF

SEQ ID NO: 119 - protein, 27 aa, Artificial sequence
  Synthetic peptide
FFSNVTWFHAIHVSGTNGTKRFDNPVL

SEQ ID NO: 120 - protein, 28 aa, Artificial sequence
  Synthetic peptide
FFSNVTWFHAIHVSGTNGTKRFDNPVLP

SEQ ID NO: 121 - protein, 29 aa, Artificial sequence
  Synthetic peptide
FFSNVTWFHAIHVSGTNGTKRFDNPVLPF

SEQ ID NO: 122 - protein, 30 aa, Artificial sequence
  Synthetic peptide
FFSNVTWFHAIHVSGTNGTKRFDNPVLPFN

SEQ ID NO: 123 - protein, 31 aa, Artificial sequence
  Synthetic peptide
FFSNVTWFHAIHVSGTNGTKRFDNPVLPFND

SEQ ID NO: 124 - protein, 137 aa, Artificial sequence
  Synthetic peptide
FFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVF

SEQ ID NO: 125 - protein, 20 aa, Artificial sequence
  Synthetic peptide
FGAGAALQIPFAMQMAYRFN

SEQ ID NO: 126 - protein, 27 aa, Artificial sequence
  Synthetic peptide
FKIYSKHTPINLVRDLPQGFSALEPLV

SEQ ID NO: 127 - protein, 28 aa, Artificial sequence
  Synthetic peptide
FKIYSKHTPINLVRDLPQGFSALEPLVD

SEQ ID NO: 128 - protein, 29 aa, Artificial sequence
  Synthetic peptide
FKIYSKHTPINLVRDLPQGFSALEPLVDL

SEQ ID NO: 129 - protein, 30 aa, Artificial sequence
  Synthetic peptide
FKIYSKHTPINLVRDLPQGFSALEPLVDLP

SEQ ID NO: 130 - protein, 69 aa, Artificial sequence
  Synthetic peptide
FKIYSKHTPINLVRDLPQGFSALEPLVDLPIGINITRFQTLLALHRSYLTPGDSSSGWTAGAAAYYVGY

SEQ ID NO: 131 - protein, 70 aa, Artificial sequence
  Synthetic peptide
FKIYSKHTPINLVRDLPQGFSALEPLVDLPIGINITRFQTLLALHRSYLTPGDSSSGWTAGAAAYYVGYL

SEQ ID NO: 132 - protein, 31 aa, Artificial sequence
  Synthetic peptide
FNGIGVTQNVLYENQKLIANQFNSAIGKIQD

SEQ ID NO: 133 - protein, 12 aa, Artificial sequence
  Synthetic peptide
FPNITNLCPFGE

SEQ ID NO: 134 - protein, 21 aa, Artificial sequence
  Synthetic peptide
FPNITNLCPFGEVFNATRFAS

SEQ ID NO: 135 - protein, 28 aa, Artificial sequence
  Synthetic peptide
FPNITNLCPFGEVFNATRFASVYAWNRK

SEQ ID NO: 136 - protein, 35 aa, Artificial sequence
  Synthetic peptide
FPNITNLCPFGEVFNATRFASVYAWNRKRISNCVA

SEQ ID NO: 137 - protein, 36 aa, Artificial sequence
  Synthetic peptide
FPNITNLCPFGEVFNATRFASVYAWNRKRISNCVAD

SEQ ID NO: 138 - protein, 37 aa, Artificial sequence
  Synthetic peptide
FPNITNLCPFGEVFNATRFASVYAWNRKRISNCVADY

SEQ ID NO: 139 - protein, 39 aa, Artificial sequence
  Synthetic peptide
FPNITNLCPFGEVFNATRFASVYAWNRKRISNCVADYSV

SEQ ID NO: 140 - protein, 47 aa, Artificial sequence
  Synthetic peptide
FPNITNLCPFGEVFNATRFASVYAWNRKRISNCVADYSVLYNSASFS

SEQ ID NO: 141 - protein, 49 aa, Artificial sequence
  Synthetic peptide
FPNITNLCPFGEVFNATRFASVYAWNRKRISNCVADYSVLYNSASFSTF

SEQ ID NO: 142 - protein, 63 aa, Artificial sequence
  Synthetic peptide
FPNITNLCPFGEVFNATRFASVYAWNRKRISNCVADYSVLYNSASFSTFKCYGVSPTKLNDLC

SEQ ID NO: 143 - protein, 28 aa, Artificial sequence
  Synthetic peptide
FSNVTWFHAIHVSGTNGTKRFDNPVLPF

SEQ ID NO: 144 - protein, 29 aa, Artificial sequence
  Synthetic peptide
FSNVTWFHAIHVSGTNGTKRFDNPVLPFN

SEQ ID NO: 145 - protein, 136 aa, Artificial sequence
  Synthetic peptide
FSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVF

SEQ ID NO: 146 - protein, 13 aa, Artificial sequence
  Synthetic peptide
GAALQIPFAMQMA

SEQ ID NO: 147 - protein, 17 aa, Artificial sequence
  Synthetic peptide
GAALQIPFAMQMAYRFN

SEQ ID NO: 148 - protein, 39 aa, Artificial sequence
  Synthetic peptide
GAALQIPFAMQMAYRFNGIGVTQNVLYENQKLIANQFNS

SEQ ID NO: 149 - protein, 42 aa, Artificial sequence
  Synthetic peptide
GAALQIPFAMQMAYRFNGIGVTQNVLYENQKLIANQFNSAIG

SEQ ID NO: 150 - protein, 43 aa, Artificial sequence
  Synthetic peptide
GAALQIPFAMQMAYRFNGIGVTQNVLYENQKLIANQFNSAIGK

SEQ ID NO: 151 - protein, 46 aa, Artificial sequence
  Synthetic peptide
GAALQIPFAMQMAYRFNGIGVTQNVLYENQKLIANQFNSAIGKIQD

SEQ ID NO: 152 - protein, 46 aa, Artificial sequence
  Synthetic peptide
GAALQIPFAMQMAYRFNGIGVTQNVLYENQKLIANQFNSVIGKIQD

SEQ ID NO: 153 - protein, 9 aa, Artificial sequence
  Synthetic peptide
GAEHVNNSY

SEQ ID NO: 154 - protein, 10 aa, Artificial sequence
  Synthetic peptide
GAEHVNNSYE

SEQ ID NO: 155 - protein, 19 aa, Artificial sequence
  Synthetic peptide
GAENSVAYSNNSIAIPTNF

SEQ ID NO: 156 - protein, 20 aa, Artificial sequence
  Synthetic peptide
GAENSVAYSNNSIAIPTNFT

SEQ ID NO: 157 - protein, 21 aa, Artificial sequence
  Synthetic peptide
GAENSVAYSNNSIAIPTNFTI

SEQ ID NO: 158 - protein, 22 aa, Artificial sequence
  Synthetic peptide
GAENSVAYSNNSIAIPTNFTIS

SEQ ID NO: 159 - protein, 19 aa, Artificial sequence
  Synthetic peptide
GAGAALQIPFAMQMAYRFN

SEQ ID NO: 160 - protein, 8 aa, Artificial sequence
  Synthetic peptide
GDCLGDIA

SEQ ID NO: 161 - protein, 39 aa, Artificial sequence
  Synthetic peptide
GINITRFQTLLALHRSYLTPGDSSSGWTAGAAAYYVGYL

SEQ ID NO: 162 - protein, 11 aa, Artificial sequence
  Synthetic peptide
GPKKSTNLVKN

SEQ ID NO: 163 - protein, 12 aa, Artificial sequence
  Synthetic peptide
GPKKSTNLVKNK

SEQ ID NO: 164 - protein, 13 aa, Artificial sequence
  Synthetic peptide
GPKKSTNLVKNKC

SEQ ID NO: 165 - protein, 27 aa, Artificial sequence
  Synthetic peptide
GSTPCNGVEGFNCYFPLQSYGFQPTNG

SEQ ID NO: 166 - protein, 20 aa, Artificial sequence
  Synthetic peptide
GVTQNVLYENQKLIANQFNS

SEQ ID NO: 167 - protein, 27 aa, Artificial sequence
  Synthetic peptide
GVTQNVLYENQKLIANQFNSAIGKIQD

SEQ ID NO: 168 - protein, 104 aa, Artificial sequence
  Synthetic peptide
GVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREF

SEQ ID NO: 169 - protein, 106 aa, Artificial sequence
  Synthetic peptide
GVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVF

SEQ ID NO: 170 - protein, 18 aa, Artificial sequence
  Synthetic peptide
HADQLTPTWRVYSTGSNV

SEQ ID NO: 171 - protein, 6 aa, Artificial sequence
  Synthetic peptide
HAPATV

SEQ ID NO: 172 - protein, 7 aa, Artificial sequence
  Synthetic peptide
HAPATVC

SEQ ID NO: 173 - protein, 26 aa, Artificial sequence
  Synthetic peptide
IAWNSNNLDSKVGGNYNYLYRLFRKS

SEQ ID NO: 174 - protein, 29 aa, Artificial sequence
  Synthetic peptide
IAWNSNNLDSKVGGNYNYLYRLFRKSNLK

SEQ ID NO: 175 - protein, 21 aa, Artificial sequence
  Synthetic peptide
IGVTQNVLYENQKLIANQFNS

SEQ ID NO: 176 - protein, 24 aa, Artificial sequence
  Synthetic peptide
IGVTQNVLYENQKLIANQFNSAIG

SEQ ID NO: 177 - protein, 25 aa, Artificial sequence
  Synthetic peptide
IGVTQNVLYENQKLIANQFNSAIGK

SEQ ID NO: 178 - protein, 27 aa, Artificial sequence
  Synthetic peptide
IGVTQNVLYENQKLIANQFNSAIGKIQ

SEQ ID NO: 179 - protein, 28 aa, Artificial sequence
  Synthetic peptide
IGVTQNVLYENQKLIANQFNSAIGKIQD

SEQ ID NO: 180 - protein, 33 aa, Artificial sequence
  Synthetic peptide
IGVTQNVLYENQKLIANQFNSAIGKIQDSLSST

SEQ ID NO: 181 - protein, 25 aa, Artificial sequence
  Synthetic peptide
IGVTQNVLYENQKLIANQFNSVIGK

SEQ ID NO: 182 - protein, 28 aa, Artificial sequence
  Synthetic peptide
IGVTQNVLYENQKLIANQFNSVIGKIQD

SEQ ID NO: 183 - protein, 7 aa, Artificial sequence
  Synthetic peptide
IHADQLT

SEQ ID NO: 184 - protein, 18 aa, Artificial sequence
  Synthetic peptide
IHADQLTPTWRVYSTGSN

SEQ ID NO: 185 - protein, 19 aa, Artificial sequence
  Synthetic peptide
IHADQLTPTWRVYSTGSNV

SEQ ID NO: 186 - protein, 10 aa, Artificial sequence
  Synthetic peptide
ISNCVADYSV

SEQ ID NO: 187 - protein, 5 aa, Artificial sequence
  Synthetic peptide
ISTEI

SEQ ID NO: 188 - protein, 36 aa, Artificial sequence
  Synthetic peptide
ISTEIYQAGSTPCNGVEGFNCYFPLQSYGFQPTNGV

SEQ ID NO: 189 - protein, 40 aa, Artificial sequence
  Synthetic peptide
ISTEIYQAGSTPCNGVEGFNCYFPLQSYGFQPTNGVGYQP

SEQ ID NO: 190 - protein, 42 aa, Artificial sequence
  Synthetic peptide
ISTEIYQAGSTPCNGVEGFNCYFPLQSYGFQPTNGVGYQPYR

SEQ ID NO: 191 - protein, 28 aa, Artificial sequence
  Synthetic peptide
ITNLCPFGEVFNATRFASVYAWNRKRIS

SEQ ID NO: 192 - protein, 36 aa, Artificial sequence
  Synthetic peptide
ITRFQTLLALHRSYLTPGDSSSGWTAGAAAYYVGYL

SEQ ID NO: 193 - protein, 4 aa, Artificial sequence
  Synthetic peptide
ITSG

SEQ ID NO: 194 - protein, 6 aa, Artificial sequence
  Synthetic peptide
ITSGWT

SEQ ID NO: 195 - protein, 7 aa, Artificial sequence
  Synthetic peptide
ITSGWTF

SEQ ID NO: 196 - protein, 30 aa, Artificial sequence
  Synthetic peptide
IYKTPPIKDCGGFNFSQILPDPSKPSKRSF

SEQ ID NO: 197 - protein, 26 aa, Artificial sequence
  Synthetic peptide
IYKTPPIKDFGGFNFSQILPDPSKPS

SEQ ID NO: 198 - protein, 27 aa, Artificial sequence
  Synthetic peptide
IYKTPPIKDFGGFNFSQILPDPSKPSK

SEQ ID NO: 199 - protein, 30 aa, Artificial sequence
  Synthetic peptide
IYKTPPIKDFGGFNFSQILPDPSKPSKRSF

SEQ ID NO: 200 - protein, 34 aa, Artificial sequence
  Synthetic peptide
IYKTPPIKDFGGFNFSQILPDPSKPSKRSFIEDL

SEQ ID NO: 201 - protein, 20 aa, Artificial sequence
  Synthetic peptide
IYSKHTPINLVRDLPQGFSA

SEQ ID NO: 202 - protein, 25 aa, Artificial sequence
  Synthetic peptide
IYSKHTPINLVRDLPQGFSALEPLV

SEQ ID NO: 203 - protein, 26 aa, Artificial sequence
  Synthetic peptide
IYSKHTPINLVRDLPQGFSALEPLVD

SEQ ID NO: 204 - protein, 27 aa, Artificial sequence
  Synthetic peptide
IYSKHTPINLVRDLPQGFSALEPLVDL

SEQ ID NO: 205 - protein, 28 aa, Artificial sequence
  Synthetic peptide
IYSKHTPINLVRDLPQGFSALEPLVDLP

SEQ ID NO: 206 - protein, 68 aa, Artificial sequence
  Synthetic peptide
IYSKHTPINLVRDLPQGFSALEPLVDLPIGINITRFQTLLALHRSYLTPGDSSSGWTAGAAAYYVGYL

SEQ ID NO: 207 - protein, 6 aa, Artificial sequence
  Synthetic peptide
KKSTNL

SEQ ID NO: 208 - protein, 9 aa, Artificial sequence
  Synthetic peptide
KKSTNLVKN

SEQ ID NO: 209 - protein, 10 aa, Artificial sequence
  Synthetic peptide
KKSTNLVKNK

SEQ ID NO: 210 - protein, 11 aa, Artificial sequence
  Synthetic peptide
KKSTNLVKNKC

SEQ ID NO: 211 - protein, 29 aa, Artificial sequence
  Synthetic peptide
KQIYKTPPIKDCGGFNFSQILPDPSKPSK

SEQ ID NO: 212 - protein, 32 aa, Artificial sequence
  Synthetic peptide
KQIYKTPPIKDCGGFNFSQILPDPSKPSKRSF

SEQ ID NO: 213 - protein, 28 aa, Artificial sequence
  Synthetic peptide
KQIYKTPPIKDFGGFNFSQILPDPSKPS

SEQ ID NO: 214 - protein, 29 aa, Artificial sequence
  Synthetic peptide
KQIYKTPPIKDFGGFNFSQILPDPSKPSK

SEQ ID NO: 215 - protein, 32 aa, Artificial sequence
  Synthetic peptide
KQIYKTPPIKDFGGFNFSQILPDPSKPSKRSF

SEQ ID NO: 216 - protein, 8 aa, Artificial sequence
  Synthetic peptide
KRISNCVA

SEQ ID NO: 217 - protein, 10 aa, Artificial sequence
  Synthetic peptide
KRISNCVADY

SEQ ID NO: 218 - protein, 16 aa, Artificial sequence
  Synthetic peptide
KTPPIKDCGGFNFSQI

SEQ ID NO: 219 - protein, 24 aa, Artificial sequence
  Synthetic peptide
KTPPIKDCGGFNFSQILPDPSKPS

SEQ ID NO: 220 - protein, 25 aa, Artificial sequence
  Synthetic peptide
KTPPIKDCGGFNFSQILPDPSKPSK

SEQ ID NO: 221 - protein, 26 aa, Artificial sequence
  Synthetic peptide
KTPPIKDCGGFNFSQILPDPSKPSKR

SEQ ID NO: 222 - protein, 28 aa, Artificial sequence
  Synthetic peptide
KTPPIKDCGGFNFSQILPDPSKPSKRSF

SEQ ID NO: 223 - protein, 14 aa, Artificial sequence
  Synthetic peptide
KTPPIKDFGGFNFS

SEQ ID NO: 224 - protein, 15 aa, Artificial sequence
  Synthetic peptide
KTPPIKDFGGFNFSQ

SEQ ID NO: 225 - protein, 16 aa, Artificial sequence
  Synthetic peptide
KTPPIKDFGGFNFSQI

SEQ ID NO: 226 - protein, 22 aa, Artificial sequence
  Synthetic peptide
KTPPIKDFGGFNFSQILPDPSK

SEQ ID NO: 227 - protein, 24 aa, Artificial sequence
  Synthetic peptide
KTPPIKDFGGFNFSQILPDPSKPS

SEQ ID NO: 228 - protein, 25 aa, Artificial sequence
  Synthetic peptide
KTPPIKDFGGFNFSQILPDPSKPSK

SEQ ID NO: 229 - protein, 26 aa, Artificial sequence
  Synthetic peptide
KTPPIKDFGGFNFSQILPDPSKPSKR

SEQ ID NO: 230 - protein, 28 aa, Artificial sequence
  Synthetic peptide
KTPPIKDFGGFNFSQILPDPSKPSKRSF

SEQ ID NO: 231 - protein, 22 aa, Artificial sequence
  Synthetic peptide
LGAENSVAYSNNSIAIPTNFTI

SEQ ID NO: 232 - protein, 41 aa, Artificial sequence
  Synthetic peptide
LKPFERDISTEIYQAGSTPCNGVEGFNCYFPLQSYGFQPTN

SEQ ID NO: 233 - protein, 42 aa, Artificial sequence
  Synthetic peptide
LKPFERDISTEIYQAGSTPCNGVEGFNCYFPLQSYGFQPTNG

SEQ ID NO: 234 - protein, 47 aa, Artificial sequence
  Synthetic peptide
LKPFERDISTEIYQAGSTPCNGVEGFNCYFPLQSYGFQPTNGVGYQP

SEQ ID NO: 235 - protein, 8 aa, Artificial sequence
  Synthetic peptide
LPDPSKPS

SEQ ID NO: 236 - protein, 32 aa, Artificial sequence
  Synthetic peptide
LPFFSNVTWFHAIHVSGTNGTKRFDNPVLPFN

SEQ ID NO: 237 - protein, 33 aa, Artificial sequence
  Synthetic peptide
LPFFSNVTWFHAIHVSGTNGTKRFDNPVLPFND

SEQ ID NO: 238 - protein, 139 aa, Artificial sequence
  Synthetic peptide
LPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVF

SEQ ID NO: 239 - protein, 141 aa, Artificial sequence
  Synthetic peptide
LPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKN

SEQ ID NO: 240 - protein, 13 aa, Artificial sequence
  Synthetic peptide
LTPTWRVYSTGSN

SEQ ID NO: 241 - protein, 14 aa, Artificial sequence
  Synthetic peptide
LTPTWRVYSTGSNV

SEQ ID NO: 242 - protein, 7 aa, Artificial sequence
  Synthetic peptide
NATRFAS

SEQ ID NO: 243 - protein, 14 aa, Artificial sequence
  Synthetic peptide
NATRFASVYAWNRK

SEQ ID NO: 244 - protein, 21 aa, Artificial sequence
  Synthetic peptide
NATRFASVYAWNRKRISNCVA

SEQ ID NO: 245 - protein, 4 aa, Artificial sequence
  Synthetic peptide
NCTE

SEQ ID NO: 246 - protein, 22 aa, Artificial sequence
  Synthetic peptide
NCVADYSVLYNSASFSTFKCYG

SEQ ID NO: 247 - protein, 30 aa, Artificial sequence
  Synthetic peptide
NCVADYSVLYNSASFSTFKCYGVSPTKLND

SEQ ID NO: 248 - protein, 31 aa, Artificial sequence
  Synthetic peptide
NCVADYSVLYNSASFSTFKCYGVSPTKLNDL

SEQ ID NO: 249 - protein, 32 aa, Artificial sequence
  Synthetic peptide
NCVADYSVLYNSASFSTFKCYGVSPTKLNDLC

SEQ ID NO: 250 - protein, 4 aa, Artificial sequence
  Synthetic peptide
NENG

SEQ ID NO: 251 - protein, 5 aa, Artificial sequence
  Synthetic peptide
NENGT

SEQ ID NO: 252 - protein, 7 aa, Artificial sequence
  Synthetic peptide
NENGTIT

SEQ ID NO: 253 - protein, 8 aa, Artificial sequence
  Synthetic peptide
NENGTITD

SEQ ID NO: 254 - protein, 37 aa, Artificial sequence
  Synthetic peptide
NITRFQTLLALHRSYLTPGDSSSGWTAGAAAYYVGYL

SEQ ID NO: 255 - protein, 26 aa, Artificial sequence
  Synthetic peptide
NLCPFGEVFNATRFASVYAWNRKRIS

SEQ ID NO: 256 - protein, 43 aa, Artificial sequence
  Synthetic peptide
NLKPFERDISTEIYQAGSTPCNGVEGFNCYFPLQSYGFQPTNG

SEQ ID NO: 257 - protein, 50 aa, Artificial sequence
  Synthetic peptide
NLKPFERDISTEIYQAGSTPCNGVEGFNCYFPLQSYGFQPTNGVGYQPYR

SEQ ID NO: 258 - protein, 52 aa, Artificial sequence
  Synthetic peptide
NLKPFERDISTEIYQAGSTPCNGVEGFNCYFPLQSYGFQPTNGVGYQPYRVV

SEQ ID NO: 259 - protein, 4 aa, Artificial sequence
  Synthetic peptide
NSAS

SEQ ID NO: 260 - protein, 5 aa, Artificial sequence
  Synthetic peptide
NSASF

SEQ ID NO: 261 - protein, 6 aa, Artificial sequence
  Synthetic peptide
NSASFS

SEQ ID NO: 262 - protein, 16 aa, Artificial sequence
  Synthetic peptide
NSNNLDSKVGGNYNYL

SEQ ID NO: 263 - protein, 18 aa, Artificial sequence
  Synthetic peptide
NSNNLDSKVGGNYNYLYR

SEQ ID NO: 264 - protein, 19 aa, Artificial sequence
  Synthetic peptide
NSNNLDSKVGGNYNYLYRL

SEQ ID NO: 265 - protein, 22 aa, Artificial sequence
  Synthetic peptide
NSNNLDSKVGGNYNYLYRLFRK

SEQ ID NO: 266 - protein, 23 aa, Artificial sequence
  Synthetic peptide
NSNNLDSKVGGNYNYLYRLFRKS

SEQ ID NO: 267 - protein, 4 aa, Artificial sequence
  Synthetic peptide
NTSN

SEQ ID NO: 268 - protein, 5 aa, Artificial sequence
  Synthetic peptide
NTSNE

SEQ ID NO: 269 - protein, 6 aa, Artificial sequence
  Synthetic peptide
NTSNEV

SEQ ID NO: 270 - protein, 121 aa, Artificial sequence
  Synthetic peptide
NVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLS

SEQ ID NO: 271 - protein, 79 aa, Artificial sequence
  Synthetic peptide
PAQEKNFTTAPAICHDGKAHFPREGVFVSNGTHWFVTQRNFYEPQIITTDNTFVSGNCDVVIGIVNNTVYDPLQPELDS

SEQ ID NO: 272 - protein, 25 aa, Artificial sequence
  Synthetic peptide
PCNGVEGFNCYFPLQSYGFQPTNGV

SEQ ID NO: 273 - protein, 7 aa, Artificial sequence
  Synthetic peptide
PDPSKPS

SEQ ID NO: 274 - protein, 8 aa, Artificial sequence
  Synthetic peptide
PDPSKPSK

SEQ ID NO: 275 - protein, 40 aa, Artificial sequence
  Synthetic peptide
PFERDISTEIYQAGSTPCNGVEGFNCYFPLQSYGFQPTNG

SEQ ID NO: 276 - protein, 40 aa, Artificial sequence
  Synthetic peptide
PIGINITRFQTLLALHRSYLTPGDSSSGWTAGAAAYYVGY

SEQ ID NO: 277 - protein, 41 aa, Artificial sequence
  Synthetic peptide
PIGINITRFQTLLALHRSYLTPGDSSSGWTAGAAAYYVGYL

SEQ ID NO: 278 - protein, 10 aa, Artificial sequence
  Synthetic peptide
PKKSTNLVKN

SEQ ID NO: 279 - protein, 11 aa, Artificial sequence
  Synthetic peptide
PKKSTNLVKNK

SEQ ID NO: 280 - protein, 11 aa, Artificial sequence
  Synthetic peptide
PNITNLCPFGE

SEQ ID NO: 281 - protein, 18 aa, Artificial sequence
  Synthetic peptide
PNITNLCPFGEVFNATRF

SEQ ID NO: 282 - protein, 24 aa, Artificial sequence
  Synthetic peptide
PNITNLCPFGEVFNATRFASVYAW

SEQ ID NO: 283 - protein, 26 aa, Artificial sequence
  Synthetic peptide
PNITNLCPFGEVFNATRFASVYAWNR

SEQ ID NO: 284 - protein, 27 aa, Artificial sequence
  Synthetic peptide
PNITNLCPFGEVFNATRFASVYAWNRK

SEQ ID NO: 285 - protein, 28 aa, Artificial sequence
  Synthetic peptide
PNITNLCPFGEVFNATRFASVYAWNRKR

SEQ ID NO: 286 - protein, 33 aa, Artificial sequence
  Synthetic peptide
PNITNLCPFGEVFNATRFASVYAWNRKRISNCV

SEQ ID NO: 287 - protein, 34 aa, Artificial sequence
  Synthetic peptide
PNITNLCPFGEVFNATRFASVYAWNRKRISNCVA

SEQ ID NO: 288 - protein, 37 aa, Artificial sequence
  Synthetic peptide
PNITNLCPFGEVFNATRFASVYAWNRKRISNCVADYS

SEQ ID NO: 289 - protein, 38 aa, Artificial sequence
  Synthetic peptide
PNITNLCPFGEVFNATRFASVYAWNRKRISNCVADYSV

SEQ ID NO: 290 - protein, 52 aa, Artificial sequence
  Synthetic peptide
PNITNLCPFGEVFNATRFASVYAWNRKRISNCVADYSVLYNSASFSTFKCYG

SEQ ID NO: 291 - protein, 59 aa, Artificial sequence
  Synthetic peptide
PNITNLCPFGEVFNATRFASVYAWNRKRISNCVADYSVLYNSASFSTFKCYGVSPTKLN

SEQ ID NO: 292 - protein, 60 aa, Artificial sequence
  Synthetic peptide
PNITNLCPFGEVFNATRFASVYAWNRKRISNCVADYSVLYNSASFSTFKCYGVSPTKLND

SEQ ID NO: 293 - protein, 61 aa, Artificial sequence
  Synthetic peptide
PNITNLCPFGEVFNATRFASVYAWNRKRISNCVADYSVLYNSASFSTFKCYGVSPTKLNDL

SEQ ID NO: 294 - protein, 187 aa, Artificial sequence
  Synthetic peptide
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFE

SEQ ID NO: 295 - protein, 5 aa, Artificial sequence
  Synthetic peptide
PQTLE

SEQ ID NO: 296 - protein, 6 aa, Artificial sequence
  Synthetic peptide
PQTLEI

SEQ ID NO: 297 - protein, 12 aa, Artificial sequence
  Synthetic peptide
PTWRVYSTGSNV

SEQ ID NO: 298 - protein, 8 aa, Artificial sequence
  Synthetic peptide
PVAIHADQ

SEQ ID NO: 299 - protein, 12 aa, Artificial sequence
  Synthetic peptide
PVAIHADQLTPT

SEQ ID NO: 300 - protein, 16 aa, Artificial sequence
  Synthetic peptide
PVAIHADQLTPTWRVY

SEQ ID NO: 301 - protein, 21 aa, Artificial sequence
  Synthetic peptide
PVAIHADQLTPTWRVYSTGSN

SEQ ID NO: 302 - protein, 22 aa, Artificial sequence
  Synthetic peptide
PVAIHADQLTPTWRVYSTGSNV

SEQ ID NO: 303 - protein, 23 aa, Artificial sequence
  Synthetic peptide
PVAIHADQLTPTWRVYSTGSNVF

SEQ ID NO: 304 - protein, 16 aa, Artificial sequence
  Synthetic peptide
QTQTNSPRRARSVASQ

SEQ ID NO: 305 - protein, 17 aa, Artificial sequence
  Synthetic peptide
QTQTNSPRRARSVASQS

SEQ ID NO: 306 - protein, 19 aa, Artificial sequence
  Synthetic peptide
QTQTNSPRRARSVASQSII

SEQ ID NO: 307 - protein, 9 aa, Artificial sequence
  Synthetic peptide
QYGDCLGDI

SEQ ID NO: 308 - protein, 37 aa, Artificial sequence
  Synthetic peptide
RDISTEIYQAGSTPCNGVEGFNCYFPLQSYGFQPTNG

SEQ ID NO: 309 - protein, 44 aa, Artificial sequence
  Synthetic peptide
RDISTEIYQAGSTPCNGVEGFNCYFPLQSYGFQPTNGVGYQPYR

SEQ ID NO: 310 - protein, 48 aa, Artificial sequence
  Synthetic peptide
RDISTEIYQAGSTPCNGVEGFNCYFPLQSYGFQPTNGVGYQPYRVVVL

SEQ ID NO: 311 - protein, 7 aa, Artificial sequence
  Synthetic peptide
RDPQTLE

SEQ ID NO: 312 - protein, 8 aa, Artificial sequence
  Synthetic peptide
RDPQTLEI

SEQ ID NO: 313 - protein, 10 aa, Artificial sequence
  Synthetic peptide
RFPNITNLCP

SEQ ID NO: 314 - protein, 21 aa, Artificial sequence
  Synthetic peptide
RFPNITNLCPFGEVFNATRFA

SEQ ID NO: 315 - protein, 22 aa, Artificial sequence
  Synthetic peptide
RFPNITNLCPFGEVFNATRFAS

SEQ ID NO: 316 - protein, 29 aa, Artificial sequence
  Synthetic peptide
RFPNITNLCPFGEVFNATRFASVYAWNRK

SEQ ID NO: 317 - protein, 35 aa, Artificial sequence
  Synthetic peptide
RFPNITNLCPFGEVFNATRFASVYAWNRKRISNCV

SEQ ID NO: 318 - protein, 36 aa, Artificial sequence
  Synthetic peptide
RFPNITNLCPFGEVFNATRFASVYAWNRKRISNCVA

SEQ ID NO: 319 - protein, 37 aa, Artificial sequence
  Synthetic peptide
RFPNITNLCPFGEVFNATRFASVYAWNRKRISNCVAD

SEQ ID NO: 320 - protein, 38 aa, Artificial sequence
  Synthetic peptide
RFPNITNLCPFGEVFNATRFASVYAWNRKRISNCVADY

SEQ ID NO: 321 - protein, 40 aa, Artificial sequence
  Synthetic peptide
RFPNITNLCPFGEVFNATRFASVYAWNRKRISNCVADYSV

SEQ ID NO: 322 - protein, 41 aa, Artificial sequence
  Synthetic peptide
RFPNITNLCPFGEVFNATRFASVYAWNRKRISNCVADYSVL

SEQ ID NO: 323 - protein, 47 aa, Artificial sequence
  Synthetic peptide
RFPNITNLCPFGEVFNATRFASVYAWNRKRISNCVADYSVLYNSASF

SEQ ID NO: 324 - protein, 48 aa, Artificial sequence
  Synthetic peptide
RFPNITNLCPFGEVFNATRFASVYAWNRKRISNCVADYSVLYNSASFS

SEQ ID NO: 325 - protein, 63 aa, Artificial sequence
  Synthetic peptide
RFPNITNLCPFGEVFNATRFASVYAWNRKRISNCVADYSVLYNSASFSTFKCYGVSPTKLNDL

SEQ ID NO: 326 - protein, 64 aa, Artificial sequence
  Synthetic peptide
RFPNITNLCPFGEVFNATRFASVYAWNRKRISNCVADYSVLYNSASFSTFKCYGVSPTKLNDLC

SEQ ID NO: 327 - protein, 9 aa, Artificial sequence
  Synthetic peptide
RVYSTGSNV

SEQ ID NO: 328 - protein, 4 aa, Artificial sequence
  Synthetic peptide
SASF

SEQ ID NO: 329 - protein, 5 aa, Artificial sequence
  Synthetic peptide
SASFS

SEQ ID NO: 330 - protein, 25 aa, Artificial sequence
  Synthetic peptide
SFVIRGDEVRQIAPGQTGKIADYNY

SEQ ID NO: 331 - protein, 26 aa, Artificial sequence
  Synthetic peptide
SFVIRGDEVRQIAPGQTGKIADYNYK

SEQ ID NO: 332 - protein, 23 aa, Artificial sequence
  Synthetic peptide
SKHTPINLVRDLPQGFSALEPLV

SEQ ID NO: 333 - protein, 24 aa, Artificial sequence
  Synthetic peptide
SKHTPINLVRDLPQGFSALEPLVD

SEQ ID NO: 334 - protein, 8 aa, Artificial sequence
  Synthetic peptide
SNCVADYS

SEQ ID NO: 335 - protein, 9 aa, Artificial sequence
  Synthetic peptide
SNCVADYSV

SEQ ID NO: 336 - protein, 15 aa, Artificial sequence
  Synthetic peptide
SNNLDSKVGGNYNYL

SEQ ID NO: 337 - protein, 16 aa, Artificial sequence
  Synthetic peptide
SNNLDSKVGGNYNYLY

SEQ ID NO: 338 - protein, 17 aa, Artificial sequence
  Synthetic peptide
SNNLDSKVGGNYNYLYR

SEQ ID NO: 339 - protein, 18 aa, Artificial sequence
  Synthetic peptide
SNNLDSKVGGNYNYLYRL

SEQ ID NO: 340 - protein, 22 aa, Artificial sequence
  Synthetic peptide
SNNLDSKVGGNYNYLYRLFRKS

SEQ ID NO: 341 - protein, 23 aa, Artificial sequence
  Synthetic peptide
SNNLDSKVGGNYNYLYRLFRKSN

SEQ ID NO: 342 - protein, 25 aa, Artificial sequence
  Synthetic peptide
SNVTWFHAIHVSGTNGTKRFDNPVL

SEQ ID NO: 343 - protein, 26 aa, Artificial sequence
  Synthetic peptide
SNVTWFHAIHVSGTNGTKRFDNPVLP

SEQ ID NO: 344 - protein, 27 aa, Artificial sequence
  Synthetic peptide
SNVTWFHAIHVSGTNGTKRFDNPVLPF

SEQ ID NO: 345 - protein, 4 aa, Artificial sequence
  Synthetic peptide
SPTK

SEQ ID NO: 346 - protein, 7 aa, Artificial sequence
  Synthetic peptide
SPTKLND

SEQ ID NO: 347 - protein, 4 aa, Artificial sequence
  Synthetic peptide
SSTA

SEQ ID NO: 348 - protein, 5 aa, Artificial sequence
  Synthetic peptide
SSTAS

SEQ ID NO: 349 - protein, 41 aa, Artificial sequence
  Synthetic peptide
STEIYQAGSTPCNGVEGFNCYFPLQSYGFQPTNGVGYQPYR

SEQ ID NO: 350 - protein, 6 aa, Artificial sequence
  Synthetic peptide
STGSNV

SEQ ID NO: 351 - protein, 26 aa, Artificial sequence
  Synthetic peptide
STPCNGVEGFNCYFPLQSYGFQPTNG

SEQ ID NO: 352 - protein, 10 aa, Artificial sequence
  Synthetic peptide
TESNKKFLPF

SEQ ID NO: 353 - protein, 11 aa, Artificial sequence
  Synthetic peptide
TESNKKFLPFQ

SEQ ID NO: 354 - protein, 26 aa, Artificial sequence
  Synthetic peptide
TEVPVAIHADQLTPTWRVYSTGSNVF

SEQ ID NO: 355 - protein, 31 aa, Artificial sequence
  Synthetic peptide
TGCVIAWNSNNLDSKVGGNYNYLYRLFRKSN

SEQ ID NO: 356 - protein, 32 aa, Artificial sequence
  Synthetic peptide
TGCVIAWNSNNLDSKVGGNYNYLYRLFRKSNL

SEQ ID NO: 357 - protein, 33 aa, Artificial sequence
  Synthetic peptide
TGCVIAWNSNNLDSKVGGNYNYLYRLFRKSNLK

SEQ ID NO: 358 - protein, 34 aa, Artificial sequence
  Synthetic peptide
TGCVIAWNSNNLDSKVGGNYNYLYRLFRKSNLKP

SEQ ID NO: 359 - protein, 84 aa, Artificial sequence
  Synthetic peptide
TGCVIAWNSNNLDSKVGGNYNYLYRLFRKSNLKPFERDISTEIYQAGSTPCNGVEGFNCYFPLQSYGFQPTNGVGYQPYRVVVL

SEQ ID NO: 360 - protein, 86 aa, Artificial sequence
  Synthetic peptide
TGCVIAWNSNNLDSKVGGNYNYLYRLFRKSNLKPFERDISTEIYQAGSTPCNGVEGFNCYFPLQSYGFQPTNGVGYQPYRVVVLSF

SEQ ID NO: 361 - protein, 4 aa, Artificial sequence
  Synthetic peptide
TGSN

SEQ ID NO: 362 - protein, 5 aa, Artificial sequence
  Synthetic peptide
TGSNV

SEQ ID NO: 363 - protein, 5 aa, Artificial sequence
  Synthetic peptide
TITSG

SEQ ID NO: 364 - protein, 84 aa, Artificial sequence
  Synthetic peptide
TLDSKTQSLLIVNNATNVVIKVCEFQFCNDPFLGVYYHKNNKSWMESEFRVYSSANNCTFEYVSQPFLMDLEGKQGNFKNLREF

SEQ ID NO: 365 - protein, 24 aa, Artificial sequence
  Synthetic peptide
TNLCPFGEVFNATRFASVYAWNRK

SEQ ID NO: 366 - protein, 6 aa, Artificial sequence
  Synthetic peptide
TNLVKN

SEQ ID NO: 367 - protein, 7 aa, Artificial sequence
  Synthetic peptide
TNLVKNK

SEQ ID NO: 368 - protein, 6 aa, Artificial sequence
  Synthetic peptide
TNTSNE

SEQ ID NO: 369 - protein, 7 aa, Artificial sequence
  Synthetic peptide
TNTSNEV

SEQ ID NO: 370 - protein, 124 aa, Artificial sequence
  Synthetic peptide
TNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFE

SEQ ID NO: 371 - protein, 12 aa, Artificial sequence
  Synthetic peptide
TPTWRVYSTGSN

SEQ ID NO: 372 - protein, 16 aa, Artificial sequence
  Synthetic peptide
TQTNSPRRARSVASQS

SEQ ID NO: 373 - protein, 32 aa, Artificial sequence
  Synthetic peptide
TRFQTLLALHRSYLTPGDSSSGWTAGAAAYYV

SEQ ID NO: 374 - protein, 33 aa, Artificial sequence
  Synthetic peptide
TRFQTLLALHRSYLTPGDSSSGWTAGAAAYYVG

SEQ ID NO: 375 - protein, 34 aa, Artificial sequence
  Synthetic peptide
TRFQTLLALHRSYLTPGDSSSGWTAGAAAYYVGY

SEQ ID NO: 376 - protein, 35 aa, Artificial sequence
  Synthetic peptide
TRFQTLLALHRSYLTPGDSSSGWTAGAAAYYVGYL

SEQ ID NO: 377 - protein, 4 aa, Artificial sequence
  Synthetic peptide
TSNE

SEQ ID NO: 378 - protein, 85 aa, Artificial sequence
  Synthetic peptide
TTLDSKTQSLLIVNNATNVVIKVCEFQFCNDPFLGVYYHKNNKSWMESEFRVYSSANNCTFEYVSQPFLMDLEGKQGNFKNLREF

SEQ ID NO: 379 - protein, 87 aa, Artificial sequence
  Synthetic peptide
TTLDSKTQSLLIVNNATNVVIKVCEFQFCNDPFLGVYYHKNNKSWMESEFRVYSSANNCTFEYVSQPFLMDLEGKQGNFKNLREFVF

SEQ ID NO: 380 - protein, 11 aa, Artificial sequence
  Synthetic peptide
TVCGPKKSTNL

SEQ ID NO: 381 - protein, 15 aa, Artificial sequence
  Synthetic peptide
TVCGPKKSTNLVKNK

SEQ ID NO: 382 - protein, 11 aa, Artificial sequence
  Synthetic peptide
TWRVYSTGSNV

SEQ ID NO: 383 - protein, 18 aa, Artificial sequence
  Synthetic peptide
VADYSVLYNSASFSTFKC

SEQ ID NO: 384 - protein, 28 aa, Artificial sequence
  Synthetic peptide
VADYSVLYNSASFSTFKCYGVSPTKLND

SEQ ID NO: 385 - protein, 29 aa, Artificial sequence
  Synthetic peptide
VADYSVLYNSASFSTFKCYGVSPTKLNDL

SEQ ID NO: 386 - protein, 9 aa, Artificial sequence
  Synthetic peptide
VAIHADQLT

SEQ ID NO: 387 - protein, 10 aa, Artificial sequence
  Synthetic peptide
VAIHADQLTP

SEQ ID NO: 388 - protein, 11 aa, Artificial sequence
  Synthetic peptide
VAIHADQLTPT

SEQ ID NO: 389 - protein, 15 aa, Artificial sequence
  Synthetic peptide
VAIHADQLTPTWRVY

SEQ ID NO: 390 - protein, 20 aa, Artificial sequence
  Synthetic peptide
VAIHADQLTPTWRVYSTGSN

SEQ ID NO: 391 - protein, 21 aa, Artificial sequence
  Synthetic peptide
VAIHADQLTPTWRVYSTGSNV

SEQ ID NO: 392 - protein, 22 aa, Artificial sequence
  Synthetic peptide
VAIHADQLTPTWRVYSTGSNVF

SEQ ID NO: 393 - protein, 24 aa, Artificial sequence
  Synthetic peptide
VAIHADQLTPTWRVYSTGSNVFQT

SEQ ID NO: 394 - protein, 13 aa, Artificial sequence
  Synthetic peptide
VCGPKKSTNLVKN

SEQ ID NO: 395 - protein, 14 aa, Artificial sequence
  Synthetic peptide
VCGPKKSTNLVKNK

SEQ ID NO: 396 - protein, 15 aa, Artificial sequence
  Synthetic peptide
VCGPKKSTNLVKNKC

SEQ ID NO: 397 - protein, 6 aa, Artificial sequence
  Synthetic peptide
VNCTEV

SEQ ID NO: 398 - protein, 80 aa, Artificial sequence
  Synthetic peptide
VPAQEKNFTTAPAICHDGKAHFPREGVFVSNGTHWFVTQRNFYEPQIITTDNTFVSGNCDVVIGIVNNTVYDPLQPELDS

SEQ ID NO: 399 - protein, 23 aa, Artificial sequence
  Synthetic peptide
VPVAIHADQLTPTWRVYSTGSNV

SEQ ID NO: 400 - protein, 10 aa, Artificial sequence
  Synthetic peptide
VRDPQTLEIL

SEQ ID NO: 401 - protein, 11 aa, Artificial sequence
  Synthetic peptide
VRFPNITNLCP

SEQ ID NO: 402 - protein, 37 aa, Artificial sequence
  Synthetic peptide
VRFPNITNLCPFGEVFNATRFASVYAWNRKRISNCVA

SEQ ID NO: 403 - protein, 38 aa, Artificial sequence
  Synthetic peptide
VRFPNITNLCPFGEVFNATRFASVYAWNRKRISNCVAD

SEQ ID NO: 404 - protein, 39 aa, Artificial sequence
  Synthetic peptide
VRFPNITNLCPFGEVFNATRFASVYAWNRKRISNCVADY

SEQ ID NO: 405 - protein, 8 aa, Artificial sequence
  Synthetic peptide
VSPTKLND

SEQ ID NO: 406 - protein, 30 aa, Artificial sequence
  Synthetic peptide
VYADSFVIRGDEVRQIAPGQTGKIADYNYK

SEQ ID NO: 407 - protein, 32 aa, Artificial sequence
  Synthetic peptide
VYADSFVIRGDEVRQIAPGQTGKIADYNYKLP

SEQ ID NO: 408 - protein, 120 aa, Artificial sequence
  Synthetic peptide
VYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLS

SEQ ID NO: 409 - protein, 121 aa, Artificial sequence
  Synthetic peptide
VYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSF

SEQ ID NO: 410 - protein, 105 aa, Artificial sequence
  Synthetic peptide
VYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVF

SEQ ID NO: 411 - protein, 8 aa, Artificial sequence
  Synthetic peptide
VYSTGSNV

SEQ ID NO: 412 - protein, 17 aa, Artificial sequence
  Synthetic peptide
WNSNNLDSKVGGNYNYL

SEQ ID NO: 413 - protein, 18 aa, Artificial sequence
  Synthetic peptide
WNSNNLDSKVGGNYNYLY

SEQ ID NO: 414 - protein, 19 aa, Artificial sequence
  Synthetic peptide
WNSNNLDSKVGGNYNYLYR

SEQ ID NO: 415 - protein, 20 aa, Artificial sequence
  Synthetic peptide
WNSNNLDSKVGGNYNYLYRL

SEQ ID NO: 416 - protein, 21 aa, Artificial sequence
  Synthetic peptide
WNSNNLDSKVGGNYNYLYRLF

SEQ ID NO: 417 - protein, 22 aa, Artificial sequence
  Synthetic peptide
WNSNNLDSKVGGNYNYLYRLFR

SEQ ID NO: 418 - protein, 23 aa, Artificial sequence
  Synthetic peptide
WNSNNLDSKVGGNYNYLYRLFRK

SEQ ID NO: 419 - protein, 24 aa, Artificial sequence
  Synthetic peptide
WNSNNLDSKVGGNYNYLYRLFRKS

SEQ ID NO: 420 - protein, 25 aa, Artificial sequence
  Synthetic peptide
WNSNNLDSKVGGNYNYLYRLFRKSN

SEQ ID NO: 421 - protein, 10 aa, Artificial sequence
  Synthetic peptide
WRVYSTGSNV

SEQ ID NO: 422 - protein, 11 aa, Artificial sequence
  Synthetic peptide
WRVYSTGSNVF

SEQ ID NO: 423 - protein, 119 aa, Artificial sequence
  Synthetic peptide
YADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLS

SEQ ID NO: 424 - protein, 13 aa, Artificial sequence
  Synthetic peptide
YAWNRKRISNCVA

SEQ ID NO: 425 - protein, 15 aa, Artificial sequence
  Synthetic peptide
YAWNRKRISNCVADY

SEQ ID NO: 426 - protein, 16 aa, Artificial sequence
  Synthetic peptide
YFASTEKSNIIRGWIF

SEQ ID NO: 427 - protein, 102 aa, Artificial sequence
  Synthetic peptide
YFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREF

SEQ ID NO: 428 - protein, 104 aa, Artificial sequence
  Synthetic peptide
YFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVF

SEQ ID NO: 429 - protein, 71 aa, Artificial sequence
  Synthetic peptide
YFKIYSKHTPINLVRDLPQGFSALEPLVDLPIGINITRFQTLLALHRSYLTPGDSSSGWTAGAAAYYVGYL

SEQ ID NO: 430 - protein, 5 aa, Artificial sequence
  Synthetic peptide
YGDCL

SEQ ID NO: 431 - protein, 26 aa, Artificial sequence
  Synthetic peptide
YKTPPIKDCGGFNFSQILPDPSKPSK

SEQ ID NO: 432 - protein, 27 aa, Artificial sequence
  Synthetic peptide
YKTPPIKDCGGFNFSQILPDPSKPSKR

SEQ ID NO: 433 - protein, 29 aa, Artificial sequence
  Synthetic peptide
YKTPPIKDCGGFNFSQILPDPSKPSKRSF

SEQ ID NO: 434 - protein, 17 aa, Artificial sequence
  Synthetic peptide
YKTPPIKDFGGFNFSQI

SEQ ID NO: 435 - protein, 23 aa, Artificial sequence
  Synthetic peptide
YKTPPIKDFGGFNFSQILPDPSK

SEQ ID NO: 436 - protein, 25 aa, Artificial sequence
  Synthetic peptide
YKTPPIKDFGGFNFSQILPDPSKPS

SEQ ID NO: 437 - protein, 26 aa, Artificial sequence
  Synthetic peptide
YKTPPIKDFGGFNFSQILPDPSKPSK

SEQ ID NO: 438 - protein, 27 aa, Artificial sequence
  Synthetic peptide
YKTPPIKDFGGFNFSQILPDPSKPSKR

SEQ ID NO: 439 - protein, 29 aa, Artificial sequence
  Synthetic peptide
YKTPPIKDFGGFNFSQILPDPSKPSKRSF

SEQ ID NO: 440 - protein, 33 aa, Artificial sequence
  Synthetic peptide
YKTPPIKDFGGFNFSQILPDPSKPSKRSFIEDL

SEQ ID NO: 441 - protein, 6 aa, Artificial sequence
  Synthetic peptide
YNSASF

SEQ ID NO: 442 - protein, 18 aa, Artificial sequence
  Synthetic peptide
YQTQTNSPRRARSVASQS

SEQ ID NO: 443 - protein, 20 aa, Artificial sequence
  Synthetic peptide
YQTQTNSPRRARSVASQSII

SEQ ID NO: 444 - protein, 7 aa, Artificial sequence
  Synthetic peptide
YSTGSNV

SEQ ID NO: 445 - protein, 8 aa, Artificial sequence
  Synthetic peptide
YSTGSNVF

SEQ ID NO: 446 - protein, 81 aa, Artificial sequence
  Synthetic peptide
YVPAQEKNFTTAPAICHDGKAHFPREGVFVSNGTHWFVTQRNFYEPQIITTDNTFVSGNCDVVIGIVNNTVYDPLQPELDS

SEQ ID NO: 447 - protein, 9 aa, Artificial sequence
  Synthetic peptide
KNLREFVFK

SEQ ID NO: 448 - protein, 9 aa, Artificial sequence
  Synthetic peptide
VSNGTHWFV

SEQ ID NO: 449 - protein, 9 aa, Artificial sequence
  Synthetic peptide
GTHWFVTQR

SEQ ID NO: 450 - protein, 9 aa, Artificial sequence
  Synthetic peptide
ITSGWTFGA

SEQ ID NO: 451 - protein, 9 aa, Artificial sequence
  Synthetic peptide
QLTPTWRVY

SEQ ID NO: 452 - protein, 9 aa, Artificial sequence
  Synthetic peptide
QQLIRAAEI

SEQ ID NO: 453 - protein, 9 aa, Artificial sequence
  Synthetic peptide
KRSFIEDLL

SEQ ID NO: 454 - protein, 9 aa, Artificial sequence
  Synthetic peptide
TLADAGFIK

SEQ ID NO: 455 - protein, 9 aa, Artificial sequence
  Synthetic peptide
RSFIEDLLF

SEQ ID NO: 456 - protein, 9 aa, Artificial sequence
  Synthetic peptide
SQSIIAYTM

SEQ ID NO: 457 - protein, 9 aa, Artificial sequence
  Synthetic peptide
SVTTEILPV

SEQ ID NO: 458 - protein, 9 aa, Artificial sequence
  Synthetic peptide
YECDIPIGA

SEQ ID NO: 459 - protein, 9 aa, Artificial sequence
  Synthetic peptide
FKNLREFVF

SEQ ID NO: 460 - protein, 9 aa, Artificial sequence
  Synthetic peptide
YRFNGIGVT

SEQ ID NO: 461 - protein, 9 aa, Artificial sequence
  Synthetic peptide
NSIAIPTNF

SEQ ID NO: 462 - protein, 9 aa, Artificial sequence
  Synthetic peptide
RAAEIRASA

SEQ ID NO: 463 - protein, 9 aa, Artificial sequence
  Synthetic peptide
NLCPFGEVF

SEQ ID NO: 464 - protein, 9 aa, Artificial sequence
  Synthetic peptide
KPFERDIST

SEQ ID NO: 465 - protein, 9 aa, Artificial sequence
  Synthetic peptide
TPTWRVYST

SEQ ID NO: 466 - protein, 9 aa, Artificial sequence
  Synthetic peptide
GEVFNATRF

SEQ ID NO: 467 - protein, 9 aa, Artificial sequence
  Synthetic peptide
TISVTTEIL

SEQ ID NO: 468 - protein, 9 aa, Artificial sequence
  Synthetic peptide
NGVEGFNCY

SEQ ID NO: 469 - protein, 9 aa, Artificial sequence
  Synthetic peptide
EVFNATRFA

SEQ ID NO: 470 - protein, 9 aa, Artificial sequence
  Synthetic peptide
GVVFLHVTY

SEQ ID NO: 471 - protein, 9 aa, Artificial sequence
  Synthetic peptide
NRALTGIAV

SEQ ID NO: 472 - protein, 9 aa, Artificial sequence
  Synthetic peptide
TSNEVAVLY

SEQ ID NO: 473 - protein, 9 aa, Artificial sequence
  Synthetic peptide
TNSFTRGVY

SEQ ID NO: 474 - protein, 9 aa, Artificial sequence
  Synthetic peptide
WTFGAGAAL

SEQ ID NO: 475 - protein, 9 aa, Artificial sequence
  Synthetic peptide
NSFTRGVYY

SEQ ID NO: 476 - protein, 9 aa, Artificial sequence
  Synthetic peptide
IAIPTNFTI

SEQ ID NO: 477 - protein, 9 aa, Artificial sequence
  Synthetic peptide
VLPFNDGVY

SEQ ID NO: 478 - protein, 9 aa, Artificial sequence
  Synthetic peptide
LQPRTFLLK

SEQ ID NO: 479 - protein, 9 aa, Artificial sequence
  Synthetic peptide
ERDISTEIY

SEQ ID NO: 480 - protein, 9 aa, Artificial sequence
  Synthetic peptide
VRFPNITNL

SEQ ID NO: 481 - protein, 9 aa, Artificial sequence
  Synthetic peptide
IPTNFTISV

SEQ ID NO: 482 - protein, 9 aa, Artificial sequence
  Synthetic peptide
IITTDNTFV

SEQ ID NO: 483 - protein, 9 aa, Artificial sequence
  Synthetic peptide
INITRFQTL

SEQ ID NO: 484 - protein, 9 aa, Artificial sequence
  Synthetic peptide
FVSNGTHWF

SEQ ID NO: 485 - protein, 9 aa, Artificial sequence
  Synthetic peptide
SVYAWNRKR

SEQ ID NO: 486 - protein, 9 aa, Artificial sequence
  Synthetic peptide
TKRFDNPVL

SEQ ID NO: 487 - protein, 9 aa, Artificial sequence
  Synthetic peptide
GTITSGWTF

SEQ ID NO: 488 - protein, 9 aa, Artificial sequence
  Synthetic peptide
NYLYRLFRK

SEQ ID NO: 489 - protein, 9 aa, Artificial sequence
  Synthetic peptide
VLSFELLHA

SEQ ID NO: 490 - protein, 9 aa, Artificial sequence
  Synthetic peptide
QIITTDNTF

SEQ ID NO: 491 - protein, 9 aa, Artificial sequence
  Synthetic peptide
CNDPFLGVY

SEQ ID NO: 492 - protein, 9 aa, Artificial sequence
  Synthetic peptide
TPCNGVEGF

SEQ ID NO: 493 - protein, 9 aa, Artificial sequence
  Synthetic peptide
DIADTTDAV

SEQ ID NO: 494 - protein, 9 aa, Artificial sequence
  Synthetic peptide
FNATRFASV

SEQ ID NO: 495 - protein, 9 aa, Artificial sequence
  Synthetic peptide
QPTESIVRF

SEQ ID NO: 496 - protein, 9 aa, Artificial sequence
  Synthetic peptide
WMESEFRVY

SEQ ID NO: 497 - protein, 9 aa, Artificial sequence
  Synthetic peptide
YQPYRVVVL

SEQ ID NO: 498 - protein, 9 aa, Artificial sequence
  Synthetic peptide
NTSNEVAVL

SEQ ID NO: 499 - protein, 9 aa, Artificial sequence
  Synthetic peptide
SANNCTFEY

SEQ ID NO: 500 - protein, 9 aa, Artificial sequence
  Synthetic peptide
YLQPRTFLL

SEQ ID NO: 501 - protein, 9 aa, Artificial sequence
  Synthetic peptide
QLNRALTGI

SEQ ID NO: 502 - protein, 9 aa, Artificial sequence
  Synthetic peptide
QRNFYEPQI

SEQ ID NO: 503 - protein, 9 aa, Artificial sequence
  Synthetic peptide
IGIVNNTVY

SEQ ID NO: 504 - protein, 9 aa, Artificial sequence
  Synthetic peptide
FIEDLLFNK

SEQ ID NO: 505 - protein, 9 aa, Artificial sequence
  Synthetic peptide
VVFLHVTYV

SEQ ID NO: 506 - protein, 9 aa, Artificial sequence
  Synthetic peptide
GVEGFNCYF

SEQ ID NO: 507 - protein, 9 aa, Artificial sequence
  Synthetic peptide
VYAWNRKRI

SEQ ID NO: 508 - protein, 9 aa, Artificial sequence
  Synthetic peptide
QSAPHGVVF

SEQ ID NO: 509 - protein, 9 aa, Artificial sequence
  Synthetic peptide
TPINLVRDL

SEQ ID NO: 510 - protein, 9 aa, Artificial sequence
  Synthetic peptide
AALQIPFAM

SEQ ID NO: 511 - protein, 9 aa, Artificial sequence
  Synthetic peptide
LPFNDGVYF

SEQ ID NO: 512 - protein, 9 aa, Artificial sequence
  Synthetic peptide
FQPTNGVGY

SEQ ID NO: 513 - protein, 9 aa, Artificial sequence
  Synthetic peptide
FLHVTYVPA

SEQ ID NO: 514 - protein, 9 aa, Artificial sequence
  Synthetic peptide
KVTLADAGF

SEQ ID NO: 515 - protein, 9 aa, Artificial sequence
  Synthetic peptide
QKFNGLTVL

SEQ ID NO: 516 - protein, 9 aa, Artificial sequence
  Synthetic peptide
SWMESEFRV

SEQ ID NO: 517 - protein, 9 aa, Artificial sequence
  Synthetic peptide
FERDISTEI

SEQ ID NO: 518 - protein, 9 aa, Artificial sequence
  Synthetic peptide
VFKNIDGYF

SEQ ID NO: 519 - protein, 9 aa, Artificial sequence
  Synthetic peptide
NATRFASVY

SEQ ID NO: 520 - protein, 9 aa, Artificial sequence
  Synthetic peptide
GAAAYYVGY

SEQ ID NO: 521 - protein, 9 aa, Artificial sequence
  Synthetic peptide
VYADSFVIR

SEQ ID NO: 522 - protein, 9 aa, Artificial sequence
  Synthetic peptide
LAGTITSGW

SEQ ID NO: 523 - protein, 9 aa, Artificial sequence
  Synthetic peptide
GVYFASTEK

SEQ ID NO: 524 - protein, 9 aa, Artificial sequence
  Synthetic peptide
LPPLLTDEM

SEQ ID NO: 525 - protein, 9 aa, Artificial sequence
  Synthetic peptide
MPPLLTDEM

SEQ ID NO: 526 - protein, 9 aa, Artificial sequence
  Synthetic peptide
ASANLAATK

SEQ ID NO: 527 - protein, 9 aa, Artificial sequence
  Synthetic peptide
AQKFNGLTL

SEQ ID NO: 528 - protein, 9 aa, Artificial sequence
  Synthetic peptide
AQKFNGLTV

SEQ ID NO: 529 - protein, 9 aa, Artificial sequence
  Synthetic peptide
VQKFNGLTV

SEQ ID NO: 530 - protein, 9 aa, Artificial sequence
  Synthetic peptide
VFQTRAGCL

SEQ ID NO: 531 - protein, 9 aa, Artificial sequence
  Synthetic peptide
ITDAVDCAL

SEQ ID NO: 532 - protein, 9 aa, Artificial sequence
  Synthetic peptide
AEVQIDRLI

SEQ ID NO: 533 - protein, 9 aa, Artificial sequence
  Synthetic peptide
YQDVNCTEV

SEQ ID NO: 534 - protein, 9 aa, Artificial sequence
  Synthetic peptide
KRFDNPVLP

SEQ ID NO: 535 - protein, 9 aa, Artificial sequence
  Synthetic peptide
AAAYYVGYL

SEQ ID NO: 536 - protein, 9 aa, Artificial sequence
  Synthetic peptide
STQDLFLPF

SEQ ID NO: 537 - protein, 9 aa, Artificial sequence
  Synthetic peptide
YLYRLFRKS

SEQ ID NO: 538 - protein, 9 aa, Artificial sequence
  Synthetic peptide
ISSVLNDIL

SEQ ID NO: 539 - protein, 9 aa, Artificial sequence
  Synthetic peptide
PFFSNVTWF

SEQ ID NO: 540 - protein, 9 aa, Artificial sequence
  Synthetic peptide
IGAGICASY

SEQ ID NO: 541 - protein, 9 aa, Artificial sequence
  Synthetic peptide
FQFCNDPFL

SEQ ID NO: 542 - protein, 9 aa, Artificial sequence
  Synthetic peptide
HWFVTQRNF

SEQ ID NO: 543 - protein, 9 aa, Artificial sequence
  Synthetic peptide
AQALNTLVK

SEQ ID NO: 544 - protein, 9 aa, Artificial sequence
  Synthetic peptide
LPFFSNVTW

SEQ ID NO: 545 - protein, 9 aa, Artificial sequence
  Synthetic peptide
RVVVLSFEL

SEQ ID NO: 546 - protein, 9 aa, Artificial sequence
  Synthetic peptide
YTNSFTRGV

SEQ ID NO: 547 - protein, 9 aa, Artificial sequence
  Synthetic peptide
FTISVTTEI

SEQ ID NO: 548 - protein, 9 aa, Artificial sequence
  Synthetic peptide
VIKVCEFQF

SEQ ID NO: 549 - protein, 9 aa, Artificial sequence
  Synthetic peptide
KMSECVLGQ

SEQ ID NO: 550 - protein, 9 aa, Artificial sequence
  Synthetic peptide
NIDGYFKIY

SEQ ID NO: 551 - protein, 9 aa, Artificial sequence
  Synthetic peptide
ARDLICVQK

SEQ ID NO: 552 - protein, 9 aa, Artificial sequence
  Synthetic peptide
NQFNSVIGK

SEQ ID NO: 553 - protein, 9 aa, Artificial sequence
  Synthetic peptide
ARDLICAQK

SEQ ID NO: 554 - protein, 9 aa, Artificial sequence
  Synthetic peptide
NQFNSAIGK

SEQ ID NO: 555 - protein, 9 aa, Artificial sequence
  Synthetic peptide
AGAALQIPF

SEQ ID NO: 556 - protein, 9 aa, Artificial sequence
  Synthetic peptide
EFQFCNDPF

SEQ ID NO: 557 - protein, 9 aa, Artificial sequence
  Synthetic peptide
PYRVVVLSF

SEQ ID NO: 558 - protein, 9 aa, Artificial sequence
  Synthetic peptide
SVLNDILSR

SEQ ID NO: 559 - protein, 9 aa, Artificial sequence
  Synthetic peptide
LEPLVDLPI

SEQ ID NO: 560 - protein, 9 aa, Artificial sequence
  Synthetic peptide
YQDLNCTEV

SEQ ID NO: 561 - protein, 9 aa, Artificial sequence
  Synthetic peptide
VLNDILSRL

SEQ ID NO: 562 - protein, 9 aa, Artificial sequence
  Synthetic peptide
RQIAPGQTG

SEQ ID NO: 563 - protein, 9 aa, Artificial sequence
  Synthetic peptide
LTDEMIAQY

SEQ ID NO: 564 - protein, 9 aa, Artificial sequence
  Synthetic peptide
VTYVPAQEK

SEQ ID NO: 565 - protein, 9 aa, Artificial sequence
  Synthetic peptide
NVYADSFVI

SEQ ID NO: 566 - protein, 9 aa, Artificial sequence
  Synthetic peptide
ILDITPCSF

SEQ ID NO: 567 - protein, 9 aa, Artificial sequence
  Synthetic peptide
RASANLAAT

SEQ ID NO: 568 - protein, 9 aa, Artificial sequence
  Synthetic peptide
NYNYLYRLF

SEQ ID NO: 569 - protein, 9 aa, Artificial sequence
  Synthetic peptide
GLTVLPPLL

SEQ ID NO: 570 - protein, 9 aa, Artificial sequence
  Synthetic peptide
RLDKVEAEV

SEQ ID NO: 571 - protein, 9 aa, Artificial sequence
  Synthetic peptide
RFDNPVLPF

SEQ ID NO: 572 - protein, 9 aa, Artificial sequence
  Synthetic peptide
NQKLIANQF

SEQ ID NO: 573 - protein, 9 aa, Artificial sequence
  Synthetic peptide
QYGDCLGDI

SEQ ID NO: 574 - protein, 9 aa, Artificial sequence
  Synthetic peptide
FLPFQQFGR

SEQ ID NO: 575 - protein, 9 aa, Artificial sequence
  Synthetic peptide
LGAENSVAY

SEQ ID NO: 576 - protein, 9 aa, Artificial sequence
  Synthetic peptide
GQTGKIADY

SEQ ID NO: 577 - protein, 9 aa, Artificial sequence
  Synthetic peptide
TLKSFTVEK

SEQ ID NO: 578 - protein, 9 aa, Artificial sequence
  Synthetic peptide
AEIRASANL

SEQ ID NO: 579 - protein, 9 aa, Artificial sequence
  Synthetic peptide
TLLALHRSY

SEQ ID NO: 580 - protein, 9 aa, Artificial sequence
  Synthetic peptide
GLTDLPPLL

SEQ ID NO: 581 - protein, 9 aa, Artificial sequence
  Synthetic peptide
QAYTNSFTR

SEQ ID NO: 582 - protein, 9 aa, Artificial sequence
  Synthetic peptide
GAEHVNNSY

SEQ ID NO: 583 - protein, 9 aa, Artificial sequence
  Synthetic peptide
FTNVYADSF

SEQ ID NO: 584 - protein, 9 aa, Artificial sequence
  Synthetic peptide
AVDCALDPL

SEQ ID NO: 585 - protein, 9 aa, Artificial sequence
  Synthetic peptide
VGGNYNYLY

SEQ ID NO: 586 - protein, 9 aa, Artificial sequence
  Synthetic peptide
FVFKNIDGY

SEQ ID NO: 587 - protein, 9 aa, Artificial sequence
  Synthetic peptide
QIAPGQTGK

SEQ ID NO: 588 - protein, 9 aa, Artificial sequence
  Synthetic peptide
TNGVGYQPY

SEQ ID NO: 589 - protein, 9 aa, Artificial sequence
  Synthetic peptide
YYPDKVFRS

SEQ ID NO: 590 - protein, 9 aa, Artificial sequence
  Synthetic peptide
TRFASVYAW

SEQ ID NO: 591 - protein, 9 aa, Artificial sequence
  Synthetic peptide
RISNCVADY

SEQ ID NO: 592 - protein, 9 aa, Artificial sequence
  Synthetic peptide
LQIPFAMQM

SEQ ID NO: 593 - protein, 9 aa, Artificial sequence
  Synthetic peptide
ECSNFLLQY

SEQ ID NO: 594 - protein, 9 aa, Artificial sequence
  Synthetic peptide
GLTLLPPLL

SEQ ID NO: 595 - protein, 9 aa, Artificial sequence
  Synthetic peptide
IEDLLFNKV

SEQ ID NO: 596 - protein, 9 aa, Artificial sequence
  Synthetic peptide
IYQTSNFRV

SEQ ID NO: 597 - protein, 9 aa, Artificial sequence
  Synthetic peptide
TRFQTLLAL

SEQ ID NO: 598 - protein, 9 aa, Artificial sequence
  Synthetic peptide
KKFLPFQQF

SEQ ID NO: 599 - protein, 9 aa, Artificial sequence
  Synthetic peptide
LTDLPPLLT

SEQ ID NO: 600 - protein, 9 aa, Artificial sequence
  Synthetic peptide
LHSTQDLFL

SEQ ID NO: 601 - protein, 9 aa, Artificial sequence
  Synthetic peptide
YSSANNCTF

SEQ ID NO: 602 - protein, 9 aa, Artificial sequence
  Synthetic peptide
FCTQLNRAL

SEQ ID NO: 603 - protein, 9 aa, Artificial sequence
  Synthetic peptide
RVDFCGKGY

SEQ ID NO: 604 - protein, 9 aa, Artificial sequence
  Synthetic peptide
QNAQALNTL

SEQ ID NO: 605 - protein, 9 aa, Artificial sequence
  Synthetic peptide
LLALHRSYL

SEQ ID NO: 606 - protein, 9 aa, Artificial sequence
  Synthetic peptide
LVRDLPQGF

SEQ ID NO: 607 - protein, 9 aa, Artificial sequence
  Synthetic peptide
SSTASALGK

SEQ ID NO: 608 - protein, 9 aa, Artificial sequence
  Synthetic peptide
SEQAYTNSF

SEQ ID NO: 609 - protein, 9 aa, Artificial sequence
  Synthetic peptide
KCYGVSPTK

SEQ ID NO: 610 - protein, 9 aa, Artificial sequence
  Synthetic peptide
HADQLTPTW

SEQ ID NO: 611 - protein, 9 aa, Artificial sequence
  Synthetic peptide
VASQSIIAY

SEQ ID NO: 612 - protein, 9 aa, Artificial sequence
  Synthetic peptide
VYDPLQPEL

SEQ ID NO: 613 - protein, 9 aa, Artificial sequence
  Synthetic peptide
VFVSNGTHW

SEQ ID NO: 614 - protein, 9 aa, Artificial sequence
  Synthetic peptide
KSNLKPFER

SEQ ID NO: 615 - protein, 9 aa, Artificial sequence
  Synthetic peptide
LQTYVTQQL

SEQ ID NO: 616 - protein, 9 aa, Artificial sequence
  Synthetic peptide
AYSNNSIAI

SEQ ID NO: 617 - protein, 9 aa, Artificial sequence
  Synthetic peptide
FRVQPTESI

SEQ ID NO: 618 - protein, 9 aa, Artificial sequence
  Synthetic peptide
DLPQGFSAL

SEQ ID NO: 619 - protein, 9 aa, Artificial sequence
  Synthetic peptide
NSASFSTFK

SEQ ID NO: 620 - protein, 9 aa, Artificial sequence
  Synthetic peptide
CVADYSVLY

SEQ ID NO: 621 - protein, 9 aa, Artificial sequence
  Synthetic peptide
STECSNFLL

SEQ ID NO: 622 - protein, 9 aa, Artificial sequence
  Synthetic peptide
LIANQFNSV

SEQ ID NO: 623 - protein, 9 aa, Artificial sequence
  Synthetic peptide
QFNSVIGKI

SEQ ID NO: 624 - protein, 9 aa, Artificial sequence
  Synthetic peptide
GSFCTQLNR

SEQ ID NO: 625 - protein, 9 aa, Artificial sequence
  Synthetic peptide
KIADYNYKL

SEQ ID NO: 626 - protein, 9 aa, Artificial sequence
  Synthetic peptide
LITGRLQSL

SEQ ID NO: 627 - protein, 9 aa, Artificial sequence
  Synthetic peptide
SEFRVYSSA

SEQ ID NO: 628 - protein, 9 aa, Artificial sequence
  Synthetic peptide
KTSVDCTMY

SEQ ID NO: 629 - protein, 9 aa, Artificial sequence
  Synthetic peptide
LLFNKVTLA

SEQ ID NO: 630 - protein, 9 aa, Artificial sequence
  Synthetic peptide
VYSTGSNVF

SEQ ID NO: 631 - protein, 9 aa, Artificial sequence
  Synthetic peptide
RRARSVASQ

SEQ ID NO: 632 - protein, 9 aa, Artificial sequence
  Synthetic peptide
QIYKTPPIK

SEQ ID NO: 633 - protein, 9 aa, Artificial sequence
  Synthetic peptide
QYGSFCTQL

SEQ ID NO: 634 - protein, 9 aa, Artificial sequence
  Synthetic peptide
IANQFNSAI

SEQ ID NO: 635 - protein, 9 aa, Artificial sequence
  Synthetic peptide
FPQSAPHGV

SEQ ID NO: 636 - protein, 9 aa, Artificial sequence
  Synthetic peptide
SIIAYTMSL

SEQ ID NO: 637 - protein, 9 aa, Artificial sequence
  Synthetic peptide
TSVDCTMYI

SEQ ID NO: 638 - protein, 9 aa, Artificial sequence
  Synthetic peptide
IAQYTSALL

SEQ ID NO: 639 - protein, 9 aa, Artificial sequence
  Synthetic peptide
APGQTGKIA

SEQ ID NO: 640 - protein, 9 aa, Artificial sequence
  Synthetic peptide
GLTVMPPLL

SEQ ID NO: 641 - protein, 9 aa, Artificial sequence
  Synthetic peptide
KQIYKTPPI

SEQ ID NO: 642 - protein, 9 aa, Artificial sequence
  Synthetic peptide
KSTNLVKNK

SEQ ID NO: 643 - protein, 9 aa, Artificial sequence
  Synthetic peptide
GVYYPDKVF

SEQ ID NO: 644 - protein, 9 aa, Artificial sequence
  Synthetic peptide
GVLTESNKK

SEQ ID NO: 645 - protein, 9 aa, Artificial sequence
  Synthetic peptide
ECSNLLLQY

SEQ ID NO: 646 - protein, 9 aa, Artificial sequence
  Synthetic peptide
LSETKCTLK

SEQ ID NO: 647 - protein, 9 aa, Artificial sequence
  Synthetic peptide
VVNQNAQAL

SEQ ID NO: 648 - protein, 9 aa, Artificial sequence
  Synthetic peptide
YVPAQEKNF

SEQ ID NO: 649 - protein, 9 aa, Artificial sequence
  Synthetic peptide
FEYVSQPFL

SEQ ID NO: 650 - protein, 9 aa, Artificial sequence
  Synthetic peptide
GIYQTSNFR

SEQ ID NO: 651 - protein, 9 aa, Artificial sequence
  Synthetic peptide
KGIYQTSNF

SEQ ID NO: 652 - protein, 9 aa, Artificial sequence
  Synthetic peptide
YRLFRKSNL

SEQ ID NO: 653 - protein, 9 aa, Artificial sequence
  Synthetic peptide
YNSASFSTF

SEQ ID NO: 654 - protein, 9 aa, Artificial sequence
  Synthetic peptide
EYVSQPFLM

SEQ ID NO: 655 - protein, 9 aa, Artificial sequence
  Synthetic peptide
ALNTLVKQL

SEQ ID NO: 656 - protein, 9 aa, Artificial sequence
  Synthetic peptide
GVYYHKNNK

SEQ ID NO: 657 - protein, 9 aa, Artificial sequence
  Synthetic peptide
MIAQYTSAL

SEQ ID NO: 658 - protein, 9 aa, Artificial sequence
  Synthetic peptide
TFEYVSQPF

SEQ ID NO: 659 - protein, 9 aa, Artificial sequence
  Synthetic peptide
ASFSTFKCY

SEQ ID NO: 660 - protein, 9 aa, Artificial sequence
  Synthetic peptide
KVFRSSVLH

SEQ ID NO: 661 - protein, 9 aa, Artificial sequence
  Synthetic peptide
NFLLQYGSF

SEQ ID NO: 662 - protein, 9 aa, Artificial sequence
  Synthetic peptide
NLLLQYGSF

SEQ ID NO: 663 - protein, 9 aa, Artificial sequence
  Synthetic peptide
KPSKRSFIE

SEQ ID NO: 664 - protein, 9 aa, Artificial sequence
  Synthetic peptide
VLYENQKLI

SEQ ID NO: 665 - protein, 9 aa, Artificial sequence
  Synthetic peptide
STECSNLLL

SEQ ID NO: 666 - protein, 9 aa, Artificial sequence
  Synthetic peptide
CYGVSPTKL

SEQ ID NO: 667 - protein, 9 aa, Artificial sequence
  Synthetic peptide
VLHSTQDLF

SEQ ID NO: 668 - protein, 9 aa, Artificial sequence
  Synthetic peptide
YQTQTNSPR

SEQ ID NO: 669 - protein, 9 aa, Artificial sequence
  Synthetic peptide
KQLSSNFGA

SEQ ID NO: 670 - protein, 9 aa, Artificial sequence
  Synthetic peptide
STASALGKL

SEQ ID NO: 671 - protein, 9 aa, Artificial sequence
  Synthetic peptide
SVLYNSASF

SEQ ID NO: 672 - protein, 9 aa, Artificial sequence
  Synthetic peptide
RVYSTGSNV

SEQ ID NO: 673 - protein, 9 aa, Artificial sequence
  Synthetic peptide
EVFAQVKQI

SEQ ID NO: 674 - protein, 9 aa, Artificial sequence
  Synthetic peptide
EILPVSMTK

SEQ ID NO: 675 - protein, 9 aa, Artificial sequence
  Synthetic peptide
SVAYSNNSI

SEQ ID NO: 676 - protein, 9 aa, Artificial sequence
  Synthetic peptide
SLSSTASAL

SEQ ID NO: 677 - protein, 9 aa, Artificial sequence
  Synthetic peptide
YFPLQSYGF

SEQ ID NO: 678 - protein, 9 aa, Artificial sequence
  Synthetic peptide
RSVASQSII

SEQ ID NO: 679 - protein, 9 aa, Artificial sequence
  Synthetic peptide
FAMQMAYRF

SEQ ID NO: 680 - protein, 9 aa, Artificial sequence
  Synthetic peptide
VFAQVKQIY

SEQ ID NO: 681 - protein, 9 aa, Artificial sequence
  Synthetic peptide
RLFRKSNLK

SEQ ID NO: 682 - protein, 9 aa, Artificial sequence
  Synthetic peptide
RLQSLQTYV

SEQ ID NO: 683 - protein, 9 aa, Artificial sequence
  Synthetic peptide
YHKNNKSWM

SEQ ID NO: 684 - protein, 9 aa, Artificial sequence
  Synthetic peptide
ELDSSEQAY

SEQ ID NO: 685 - protein, 9 aa, Artificial sequence
  Synthetic peptide
LVKNKCVNF

SEQ ID NO: 686 - protein, 9 aa, Artificial sequence
  Synthetic peptide
HLMSFPQSA

SEQ ID NO: 687 - protein, 9 aa, Artificial sequence
  Synthetic peptide
LGQSKRVDF

SEQ ID NO: 688 - protein, 9 aa, Artificial sequence
  Synthetic peptide
KIYSKHTPI

SEQ ID NO: 689 - protein, 9 aa, Artificial sequence
  Synthetic peptide
ARSVASQSI

SEQ ID NO: 690 - protein, 9 aa, Artificial sequence
  Synthetic peptide
IPFAMQMAY

SEQ ID NO: 691 - protein, 9 aa, Artificial sequence
  Synthetic peptide
ESNKKFLPF

SEQ ID NO: 692 - protein, 9 aa, Artificial sequence
  Synthetic peptide
VLTESNKKF

SEQ ID NO: 693 - protein, 9 aa, Artificial sequence
  Synthetic peptide
GRLQSLQTY

SEQ ID NO: 694 - protein, 9 aa, Artificial sequence
  Synthetic peptide
ETKCTLKSF

SEQ ID NO: 695 - protein, 9 aa, Artificial sequence
  Synthetic peptide
TPGDSSSGW

SEQ ID NO: 696 - protein, 9 aa, Artificial sequence
  Synthetic peptide
GPKKSTNLV

SEQ ID NO: 697 - protein, 9 aa, Artificial sequence
  Synthetic peptide
FRKSNLKPF

SEQ ID NO: 698 - protein, 9 aa, Artificial sequence
  Synthetic peptide
LVKQLSSNF

SEQ ID NO: 699 - protein, 9 aa, Artificial sequence
  Synthetic peptide
YYHKNNKSW

SEQ ID NO: 700 - protein, 9 aa, Artificial sequence
  Synthetic peptide
TLDSKTQSL

SEQ ID NO: 701 - protein, 15 aa, Artificial sequence
  Synthetic peptide
ASVYAWNRKRISNCV

SEQ ID NO: 702 - protein, 15 aa, Artificial sequence
  Synthetic peptide
ATRFASVYAWNRKRI

SEQ ID NO: 703 - protein, 15 aa, Artificial sequence
  Synthetic peptide
CAQKFNGLTLLPPLL

SEQ ID NO: 704 - protein, 15 aa, Artificial sequence
  Synthetic peptide
CSNLLLQYGSFCTQL

SEQ ID NO: 705 - protein, 15 aa, Artificial sequence
  Synthetic peptide
CTFEYVSQPFLMDLE

SEQ ID NO: 706 - protein, 15 aa, Artificial sequence
  Synthetic peptide
DEMIAQYTSALLAGT

SEQ ID NO: 707 - protein, 15 aa, Artificial sequence
  Synthetic peptide
ECSNLLLQYGSFCTQ

SEQ ID NO: 708 - protein, 15 aa, Artificial sequence
  Synthetic peptide
EMIAQYTSALLAGTI

SEQ ID NO: 709 - protein, 15 aa, Artificial sequence
  Synthetic peptide
FASVYAWNRKRISNC

SEQ ID NO: 710 - protein, 15 aa, Artificial sequence
  Synthetic peptide
FLPFFSNVTWFHAIH

SEQ ID NO: 711 - protein, 15 aa, Artificial sequence
  Synthetic peptide
GGNYNYLYRLFRKSN

SEQ ID NO: 712 - protein, 15 aa, Artificial sequence
  Synthetic peptide
GNCDVVIGIVNNTVY

SEQ ID NO: 713 - protein, 15 aa, Artificial sequence
  Synthetic peptide
GNYNYLYRLFRKSNL

SEQ ID NO: 714 - protein, 15 aa, Artificial sequence
  Synthetic peptide
GYQPYRVVVLSFELL

SEQ ID NO: 715 - protein, 15 aa, Artificial sequence
  Synthetic peptide
IRAAEIRASANLAAT

SEQ ID NO: 716 - protein, 15 aa, Artificial sequence
  Synthetic peptide
KPFERDISTEIYQAG

SEQ ID NO: 717 - protein, 15 aa, Artificial sequence
  Synthetic peptide
KVGGNYNYLYRLFRK

SEQ ID NO: 718 - protein, 15 aa, Artificial sequence
  Synthetic peptide
LFLPFFSNVTWFHAI

SEQ ID NO: 719 - protein, 15 aa, Artificial sequence
  Synthetic peptide
LIRAAEIRASANLAA

SEQ ID NO: 720 - protein, 15 aa, Artificial sequence
  Synthetic peptide
LKPFERDISTEIYQA

SEQ ID NO: 721 - protein, 15 aa, Artificial sequence
  Synthetic peptide
LLLQYGSFCTQLNRA

SEQ ID NO: 722 - protein, 15 aa, Artificial sequence
  Synthetic peptide
LLQYGSFCTQLNRAL

SEQ ID NO: 723 - protein, 15 aa, Artificial sequence
  Synthetic peptide
LQYGSFCTQLNRALT

SEQ ID NO: 724 - protein, 15 aa, Artificial sequence
  Synthetic peptide
NATRFASVYAWNRKR

SEQ ID NO: 725 - protein, 15 aa, Artificial sequence
  Synthetic peptide
NLKPFERDISTEIYQ

SEQ ID NO: 726 - protein, 15 aa, Artificial sequence
  Synthetic peptide
NLLLQYGSFCTQLNR

SEQ ID NO: 727 - protein, 15 aa, Artificial sequence
  Synthetic peptide
PREGVFVSNGTHWFV

SEQ ID NO: 728 - protein, 15 aa, Artificial sequence
  Synthetic peptide
PTNFTISVTTEILPV

SEQ ID NO: 729 - protein, 15 aa, Artificial sequence
  Synthetic peptide
PYRVVVLSFELLHAP

SEQ ID NO: 730 - protein, 15 aa, Artificial sequence
  Synthetic peptide
QLIRAAEIRASANLA

SEQ ID NO: 731 - protein, 15 aa, Artificial sequence
  Synthetic peptide
QPYRVVVLSFELLHA

SEQ ID NO: 732 - protein, 15 aa, Artificial sequence
  Synthetic peptide
QQLIRAAEIRASANL

SEQ ID NO: 733 - protein, 15 aa, Artificial sequence
  Synthetic peptide
RAAEIRASANLAATK

SEQ ID NO: 734 - protein, 15 aa, Artificial sequence
  Synthetic peptide
RFASVYAWNRKRISN

SEQ ID NO: 735 - protein, 15 aa, Artificial sequence
  Synthetic peptide
RVVVLSFELLHAPAT

SEQ ID NO: 736 - protein, 15 aa, Artificial sequence
  Synthetic peptide
SKVGGNYNYLYRLFR

SEQ ID NO: 737 - protein, 15 aa, Artificial sequence
  Synthetic peptide
SNLKPFERDISTEIY

SEQ ID NO: 738 - protein, 15 aa, Artificial sequence
  Synthetic peptide
SNLLLQYGSFCTQLN

SEQ ID NO: 739 - protein, 15 aa, Artificial sequence
  Synthetic peptide
SVLYNSASFSTFKCY

SEQ ID NO: 740 - protein, 15 aa, Artificial sequence
  Synthetic peptide
SVYAWNRKRISNCVA

SEQ ID NO: 741 - protein, 15 aa, Artificial sequence
  Synthetic peptide
TDEMIAQYTSALLAG

SEQ ID NO: 742 - protein, 15 aa, Artificial sequence
  Synthetic peptide
TECSNLLLQYGSFCT

SEQ ID NO: 743 - protein, 15 aa, Artificial sequence
  Synthetic peptide
TFEYVSQPFLMDLEG

SEQ ID NO: 744 - protein, 15 aa, Artificial sequence
  Synthetic peptide
TQQLIRAAEIRASAN

SEQ ID NO: 745 - protein, 15 aa, Artificial sequence
  Synthetic peptide
TRFASVYAWNRKRIS

SEQ ID NO: 746 - protein, 15 aa, Artificial sequence
  Synthetic peptide
VFNATRFASVYAWNR

SEQ ID NO: 747 - protein, 15 aa, Artificial sequence
  Synthetic peptide
VGGNYNYLYRLFRKS

SEQ ID NO: 748 - protein, 15 aa, Artificial sequence
  Synthetic peptide
VLSFELLHAPATVCG

SEQ ID NO: 749 - protein, 15 aa, Artificial sequence
  Synthetic peptide
VTQQLIRAAEIRASA

SEQ ID NO: 750 - protein, 15 aa, Artificial sequence
  Synthetic peptide
VVLSFELLHAPATVC

SEQ ID NO: 751 - protein, 15 aa, Artificial sequence
  Synthetic peptide
YLYRLFRKSNLKPFE

SEQ ID NO: 752 - protein, 15 aa, Artificial sequence
  Synthetic peptide
YQPYRVVVLSFELLH

SEQ ID NO: 753 - protein, 15 aa, Artificial sequence
  Synthetic peptide
YRVVVLSFELLHAPA

SEQ ID NO: 754 - protein, 15 aa, Artificial sequence
  Synthetic peptide
YSVLYNSASFSTFKC

SEQ ID NO: 755 - protein, 15 aa, Artificial sequence
  Synthetic peptide
YVTQQLIRAAEIRAS

SEQ ID NO: 756 - protein, 15 aa, Artificial sequence
  Synthetic peptide
CPFGEVFNATRFASV

SEQ ID NO: 757 - protein, 15 aa, Artificial sequence
  Synthetic peptide
CSNFLLQYGSFCTQL

SEQ ID NO: 758 - protein, 15 aa, Artificial sequence
  Synthetic peptide
DLFLPFFSNVTWFHA

SEQ ID NO: 759 - protein, 15 aa, Artificial sequence
  Synthetic peptide
ECSNFLLQYGSFCTQ

SEQ ID NO: 760 - protein, 15 aa, Artificial sequence
  Synthetic peptide
ENQKLIANQFNSAIG

SEQ ID NO: 761 - protein, 15 aa, Artificial sequence
  Synthetic peptide
ENQKLIANQFNSVIG

SEQ ID NO: 762 - protein, 15 aa, Artificial sequence
  Synthetic peptide
EVFNATRFASVYAWN

SEQ ID NO: 763 - protein, 15 aa, Artificial sequence
  Synthetic peptide
FGEVFNATRFASVYA

SEQ ID NO: 764 - protein, 15 aa, Artificial sequence
  Synthetic peptide
FLLQYGSFCTQLNRA

SEQ ID NO: 765 - protein, 15 aa, Artificial sequence
  Synthetic peptide
FQTLLALHRSYLTPG

SEQ ID NO: 766 - protein, 15 aa, Artificial sequence
  Synthetic peptide
GCVIAWNSNNLDSKV

SEQ ID NO: 767 - protein, 15 aa, Artificial sequence
  Synthetic peptide
GEVFNATRFASVYAW

SEQ ID NO: 768 - protein, 15 aa, Artificial sequence
  Synthetic peptide
GINITRFQTLLALHR

SEQ ID NO: 769 - protein, 15 aa, Artificial sequence
  Synthetic peptide
GWTAGAAAYYVGYLQ

SEQ ID NO: 770 - protein, 15 aa, Artificial sequence
  Synthetic peptide
HSTQDLFLPFFSNVT

SEQ ID NO: 771 - protein, 15 aa, Artificial sequence
  Synthetic peptide
IPIGAGICASYQTQT

SEQ ID NO: 772 - protein, 15 aa, Artificial sequence
  Synthetic peptide
ITRFQTLLALHRSYL

SEQ ID NO: 773 - protein, 15 aa, Artificial sequence
  Synthetic peptide
LLIVNNATNVVIKVC

SEQ ID NO: 774 - protein, 15 aa, Artificial sequence
  Synthetic peptide
LLTDEMIAQYTSALL

SEQ ID NO: 775 - protein, 15 aa, Artificial sequence
  Synthetic peptide
LNRALTGIAVEQDKN

SEQ ID NO: 776 - protein, 15 aa, Artificial sequence
  Synthetic peptide
LTDEMIAQYTSALLA

SEQ ID NO: 777 - protein, 15 aa, Artificial sequence
  Synthetic peptide
NFLLQYGSFCTQLNR

SEQ ID NO: 778 - protein, 15 aa, Artificial sequence
  Synthetic peptide
NITRFQTLLALHRSY

SEQ ID NO: 779 - protein, 15 aa, Artificial sequence
  Synthetic peptide
NQKLIANQFNSAIGK

SEQ ID NO: 780 - protein, 15 aa, Artificial sequence
  Synthetic peptide
NQKLIANQFNSVIGK

SEQ ID NO: 781 - protein, 15 aa, Artificial sequence
  Synthetic peptide
PFGEVFNATRFASVY

SEQ ID NO: 782 - protein, 15 aa, Artificial sequence
  Synthetic peptide
PRRARSVASQSIIAY

SEQ ID NO: 783 - protein, 15 aa, Artificial sequence
  Synthetic peptide
QKLIANQFNSAIGKI

SEQ ID NO: 784 - protein, 15 aa, Artificial sequence
  Synthetic peptide
QLNRALTGIAVEQDK

SEQ ID NO: 785 - protein, 15 aa, Artificial sequence
  Synthetic peptide
QTLLALHRSYLTPGD

SEQ ID NO: 786 - protein, 15 aa, Artificial sequence
  Synthetic peptide
RGVYYPDKVFRSSVL

SEQ ID NO: 787 - protein, 15 aa, Artificial sequence
  Synthetic peptide
RRARSVASQSIIAYT

SEQ ID NO: 788 - protein, 15 aa, Artificial sequence
  Synthetic peptide
SGWTAGAAAYYVGYL

SEQ ID NO: 789 - protein, 15 aa, Artificial sequence
  Synthetic peptide
SLLIVNNATNVVIKV

SEQ ID NO: 790 - protein, 15 aa, Artificial sequence
  Synthetic peptide
SNFLLQYGSFCTQLN

SEQ ID NO: 791 - protein, 15 aa, Artificial sequence
  Synthetic peptide
SPRRARSVASQSIIA

SEQ ID NO: 792 - protein, 15 aa, Artificial sequence
  Synthetic peptide
SSGWTAGAAAYYVGY

SEQ ID NO: 793 - protein, 15 aa, Artificial sequence
  Synthetic peptide
SSSGWTAGAAAYYVG

SEQ ID NO: 794 - protein, 15 aa, Artificial sequence
  Synthetic peptide
STQDLFLPFFSNVTW

SEQ ID NO: 795 - protein, 15 aa, Artificial sequence
  Synthetic peptide
TECSNFLLQYGSFCT

SEQ ID NO: 796 - protein, 15 aa, Artificial sequence
  Synthetic peptide
TLLALHRSYLTPGDS

SEQ ID NO: 797 - protein, 15 aa, Artificial sequence
  Synthetic peptide
TQLNRALTGIAVEQD

SEQ ID NO: 798 - protein, 15 aa, Artificial sequence
  Synthetic peptide
TRFQTLLALHRSYLT

SEQ ID NO: 799 - protein, 15 aa, Artificial sequence
  Synthetic peptide
TRGVYYPDKVFRSSV

SEQ ID NO: 800 - protein, 15 aa, Artificial sequence
  Synthetic peptide
VADYSVLYNSASFST

SEQ ID NO: 801 - protein, 15 aa, Artificial sequence
  Synthetic peptide
VVVLSFELLHAPATV

SEQ ID NO: 802 - protein, 15 aa, Artificial sequence
  Synthetic peptide
YENQKLIANQFNSAI

SEQ ID NO: 803 - protein, 15 aa, Artificial sequence
  Synthetic peptide
YENQKLIANQFNSVI

SEQ ID NO: 804 - protein, 15 aa, Artificial sequence
  Synthetic peptide
DSSSGWTAGAAAYYV

SEQ ID NO: 805 - protein, 15 aa, Artificial sequence
  Synthetic peptide
NGLTVMPPLLTDEMI

SEQ ID NO: 806 - protein, 15 aa, Artificial sequence
  Synthetic peptide
AAEIRASANLAATKM

SEQ ID NO: 807 - protein, 15 aa, Artificial sequence
  Synthetic peptide
AALQIPFAMQMAYRF

SEQ ID NO: 808 - protein, 15 aa, Artificial sequence
  Synthetic peptide
ADSFVIRGDEVRQIA

SEQ ID NO: 809 - protein, 15 aa, Artificial sequence
  Synthetic peptide
AEIRASANLAATKMS

SEQ ID NO: 810 - protein, 15 aa, Artificial sequence
  Synthetic peptide
AIPTNFTISVTTEIL

SEQ ID NO: 811 - protein, 15 aa, Artificial sequence
  Synthetic peptide
ALQIPFAMQMAYRFN

SEQ ID NO: 812 - protein, 15 aa, Artificial sequence
  Synthetic peptide
AMQMAYRFNGIGVTQ

SEQ ID NO: 813 - protein, 15 aa, Artificial sequence
  Synthetic peptide
ANNCTFEYVSQPFLM

SEQ ID NO: 814 - protein, 15 aa, Artificial sequence
  Synthetic peptide
AQKFNGLTVLPPLLT

SEQ ID NO: 815 - protein, 15 aa, Artificial sequence
  Synthetic peptide
AQKFNGLTVMPPLLT

SEQ ID NO: 816 - protein, 15 aa, Artificial sequence
  Synthetic peptide
CDIPIGAGICASYQT

SEQ ID NO: 817 - protein, 15 aa, Artificial sequence
  Synthetic peptide
CDVVIGIVNNTVYDP

SEQ ID NO: 818 - protein, 15 aa, Artificial sequence
  Synthetic peptide
DFGGFNFSQILPDPS

SEQ ID NO: 819 - protein, 15 aa, Artificial sequence
  Synthetic peptide
DGYFKIYSKHTPINL

SEQ ID NO: 820 - protein, 15 aa, Artificial sequence
  Synthetic peptide
DSFVIRGDEVRQIAP

SEQ ID NO: 821 - protein, 15 aa, Artificial sequence
  Synthetic peptide
DVVIGIVNNTVYDPL

SEQ ID NO: 822 - protein, 15 aa, Artificial sequence
  Synthetic peptide
DYSVLYNSASFSTFK

SEQ ID NO: 823 - protein, 15 aa, Artificial sequence
  Synthetic peptide
EFVFKNIDGYFKIYS

SEQ ID NO: 824 - protein, 15 aa, Artificial sequence
  Synthetic peptide
EGVFVSNGTHWFVTQ

SEQ ID NO: 825 - protein, 15 aa, Artificial sequence
  Synthetic peptide
EIRASANLAATKMSE

SEQ ID NO: 826 - protein, 15 aa, Artificial sequence
  Synthetic peptide
FELLHAPATVCGPKK

SEQ ID NO: 827 - protein, 15 aa, Artificial sequence
  Synthetic peptide
FIEDLLFNKVTLADA

SEQ ID NO: 828 - protein, 15 aa, Artificial sequence
  Synthetic peptide
FKNIDGYFKIYSKHT

SEQ ID NO: 829 - protein, 15 aa, Artificial sequence
  Synthetic peptide
FKNLREFVFKNIDGY

SEQ ID NO: 830 - protein, 15 aa, Artificial sequence
  Synthetic peptide
FVFKNIDGYFKIYSK

SEQ ID NO: 831 - protein, 15 aa, Artificial sequence
  Synthetic peptide
FVIRGDEVRQIAPGQ

SEQ ID NO: 832 - protein, 15 aa, Artificial sequence
  Synthetic peptide
GAALQIPFAMQMAYR

SEQ ID NO: 833 - protein, 15 aa, Artificial sequence
  Synthetic peptide
GNFKNLREFVFKNID

SEQ ID NO: 834 - protein, 15 aa, Artificial sequence
  Synthetic peptide
GTHWFVTQRNFYEPQ

SEQ ID NO: 835 - protein, 15 aa, Artificial sequence
  Synthetic peptide
GVFVSNGTHWFVTQR

SEQ ID NO: 836 - protein, 15 aa, Artificial sequence
  Synthetic peptide
IAIPTNFTISVTTEI

SEQ ID NO: 837 - protein, 15 aa, Artificial sequence
  Synthetic peptide
IDGYFKIYSKHTPIN

SEQ ID NO: 838 - protein, 15 aa, Artificial sequence
  Synthetic peptide
IEDLLFNKVTLADAG

SEQ ID NO: 839 - protein, 15 aa, Artificial sequence
  Synthetic peptide
IKDFGGFNFSQILPD

SEQ ID NO: 840 - protein, 15 aa, Artificial sequence
  Synthetic peptide
IPTNFTISVTTEILP

SEQ ID NO: 841 - protein, 15 aa, Artificial sequence
  Synthetic peptide
KNIDGYFKIYSKHTP

SEQ ID NO: 842 - protein, 15 aa, Artificial sequence
  Synthetic peptide
KQLSSNFGAISSVLN

SEQ ID NO: 843 - protein, 15 aa, Artificial sequence
  Synthetic peptide
KQLSSNSGAISSVLN

SEQ ID NO: 844 - protein, 15 aa, Artificial sequence
  Synthetic peptide
KRSFIEDLLFNKVTL

SEQ ID NO: 845 - protein, 15 aa, Artificial sequence
  Synthetic peptide
KSNLKPFERDISTEI

SEQ ID NO: 846 - protein, 15 aa, Artificial sequence
  Synthetic peptide
LQIPFAMQMAYRFNG

SEQ ID NO: 847 - protein, 15 aa, Artificial sequence
  Synthetic peptide
LREFVFKNIDGYFKI

SEQ ID NO: 848 - protein, 15 aa, Artificial sequence
  Synthetic peptide
LSFELLHAPATVCGP

SEQ ID NO: 849 - protein, 15 aa, Artificial sequence
  Synthetic peptide
LVKQLSSNFGAISSV

SEQ ID NO: 850 - protein, 15 aa, Artificial sequence
  Synthetic peptide
LVKQLSSNSGAISSV

SEQ ID NO: 851 - protein, 15 aa, Artificial sequence
  Synthetic peptide
MQMAYRFNGIGVTQN

SEQ ID NO: 852 - protein, 15 aa, Artificial sequence
  Synthetic peptide
NCDVVIGIVNNTVYD

SEQ ID NO: 853 - protein, 15 aa, Artificial sequence
  Synthetic peptide
NCTFEYVSQPFLMDL

SEQ ID NO: 854 - protein, 15 aa, Artificial sequence
  Synthetic peptide
NFKNLREFVFKNIDG

SEQ ID NO: 855 - protein, 15 aa, Artificial sequence
  Synthetic peptide
NIDGYFKIYSKHTPI

SEQ ID NO: 856 - protein, 15 aa, Artificial sequence
  Synthetic peptide
NLREFVFKNIDGYFK

SEQ ID NO: 857 - protein, 15 aa, Artificial sequence
  Synthetic peptide
NNCTFEYVSQPFLMD

SEQ ID NO: 858 - protein, 15 aa, Artificial sequence
  Synthetic peptide
NTLVKQLSSNFGAIS

SEQ ID NO: 859 - protein, 15 aa, Artificial sequence
  Synthetic peptide
NTLVKQLSSNSGAIS

SEQ ID NO: 860 - protein, 15 aa, Artificial sequence
  Synthetic peptide
NYLYRLFRKSNLKPF

SEQ ID NO: 861 - protein, 15 aa, Artificial sequence
  Synthetic peptide
NYNYLYRLFRKSNLK

SEQ ID NO: 862 - protein, 15 aa, Artificial sequence
  Synthetic peptide
PIKDFGGFNFSQILP

SEQ ID NO: 863 - protein, 15 aa, Artificial sequence
  Synthetic peptide
PSKRSFIEDLLFNKV

SEQ ID NO: 864 - protein, 15 aa, Artificial sequence
  Synthetic peptide
QGNFKNLREFVFKNI

SEQ ID NO: 865 - protein, 15 aa, Artificial sequence
  Synthetic peptide
QIPFAMQMAYRFNGI

SEQ ID NO: 866 - protein, 15 aa, Artificial sequence
  Synthetic peptide
QKFNGLTVLPPLLTD

SEQ ID NO: 867 - protein, 15 aa, Artificial sequence
  Synthetic peptide
QKFNGLTVMPPLLTD

SEQ ID NO: 868 - protein, 15 aa, Artificial sequence
  Synthetic peptide
QMAYRFNGIGVTQNV

SEQ ID NO: 869 - protein, 15 aa, Artificial sequence
  Synthetic peptide
QYGSFCTQLNRALTG

SEQ ID NO: 870 - protein, 15 aa, Artificial sequence
  Synthetic peptide
REFVFKNIDGYFKIY

SEQ ID NO: 871 - protein, 15 aa, Artificial sequence
  Synthetic peptide
REGVFVSNGTHWFVT

SEQ ID NO: 872 - protein, 15 aa, Artificial sequence
  Synthetic peptide
RSFIEDLLFNKVTLA

SEQ ID NO: 873 - protein, 15 aa, Artificial sequence
  Synthetic peptide
SFELLHAPATVCGPK

SEQ ID NO: 874 - protein, 15 aa, Artificial sequence
  Synthetic peptide
SFIEDLLFNKVTLAD

SEQ ID NO: 875 - protein, 15 aa, Artificial sequence
  Synthetic peptide
SFVIRGDEVRQIAPG

SEQ ID NO: 876 - protein, 15 aa, Artificial sequence
  Synthetic peptide
SKRSFIEDLLFNKVT

SEQ ID NO: 877 - protein, 15 aa, Artificial sequence
  Synthetic peptide
THWFVTQRNFYEPQI

SEQ ID NO: 878 - protein, 15 aa, Artificial sequence
  Synthetic peptide
TNFTISVTTEILPVS

SEQ ID NO: 879 - protein, 15 aa, Artificial sequence
  Synthetic peptide
VEGFNCYFPLQSYGF

SEQ ID NO: 880 - protein, 15 aa, Artificial sequence
  Synthetic peptide
VFKNIDGYFKIYSKH

SEQ ID NO: 881 - protein, 15 aa, Artificial sequence
  Synthetic peptide
VFVSNGTHWFVTQRN

SEQ ID NO: 882 - protein, 15 aa, Artificial sequence
  Synthetic peptide
VKQLSSNFGAISSVL

SEQ ID NO: 883 - protein, 15 aa, Artificial sequence
  Synthetic peptide
VKQLSSNSGAISSVL

SEQ ID NO: 884 - protein, 15 aa, Artificial sequence
  Synthetic peptide
VQKFNGLTVLPPLLT

SEQ ID NO: 885 - protein, 15 aa, Artificial sequence
  Synthetic peptide
YNYLYRLFRKSNLKP

SEQ ID NO: 886 - protein, 15 aa, Artificial sequence
  Synthetic peptide
AQKFNGLTLLPPLLT

SEQ ID NO: 887 - protein, 15 aa, Artificial sequence
  Synthetic peptide
ECDIPIGAGICASYQ

SEQ ID NO: 888 - protein, 15 aa, Artificial sequence
  Synthetic peptide
GWTFGAGAALQIPFA

SEQ ID NO: 889 - protein, 15 aa, Artificial sequence
  Synthetic peptide
IVNNATNVVIKVCEF

SEQ ID NO: 890 - protein, 15 aa, Artificial sequence
  Synthetic peptide
KFNGLTLLPPLLTDE

SEQ ID NO: 891 - protein, 15 aa, Artificial sequence
  Synthetic peptide
KFNGLTVLPPLLTDE

SEQ ID NO: 892 - protein, 15 aa, Artificial sequence
  Synthetic peptide
KFNGLTVMPPLLTDE

SEQ ID NO: 893 - protein, 15 aa, Artificial sequence
  Synthetic peptide
LIVNNATNVVIKVCE

SEQ ID NO: 894 - protein, 15 aa, Artificial sequence
  Synthetic peptide
QKFNGLTLLPPLLTD

SEQ ID NO: 895 - protein, 15 aa, Artificial sequence
  Synthetic peptide
SGNCDVVIGIVNNTV

SEQ ID NO: 896 - protein, 15 aa, Artificial sequence
  Synthetic peptide
WTFGAGAALQIPFAM

SEQ ID NO: 897 - protein, 15 aa, Artificial sequence
  Synthetic peptide
YADSFVIRGDEVRQI

SEQ ID NO: 898 - protein, 15 aa, Artificial sequence
  Synthetic peptide
YECDIPIGAGICASY

SEQ ID NO: 899 - protein, 15 aa, Artificial sequence
  Synthetic peptide
ADYSVLYNSASFSTF

SEQ ID NO: 900 - protein, 15 aa, Artificial sequence
  Synthetic peptide
AEVQIDRLITGRLQS

SEQ ID NO: 901 - protein, 15 aa, Artificial sequence
  Synthetic peptide
ALNTLVKQLSSNFGA

SEQ ID NO: 902 - protein, 15 aa, Artificial sequence
  Synthetic peptide
ALNTLVKQLSSNSGA

SEQ ID NO: 903 - protein, 15 aa, Artificial sequence
  Synthetic peptide
AQALNTLVKQLSSNF

SEQ ID NO: 904 - protein, 15 aa, Artificial sequence
  Synthetic peptide
CGGFNFSQILPDPSK

SEQ ID NO: 905 - protein, 15 aa, Artificial sequence
  Synthetic peptide
DCGGFNFSQILPDPS

SEQ ID NO: 906 - protein, 15 aa, Artificial sequence
  Synthetic peptide
DIPIGAGICASYQTQ

SEQ ID NO: 907 - protein, 15 aa, Artificial sequence
  Synthetic peptide
EAEVQIDRLITGRLQ

SEQ ID NO: 908 - protein, 15 aa, Artificial sequence
  Synthetic peptide
EDLLFNKVTLADAGF

SEQ ID NO: 909 - protein, 15 aa, Artificial sequence
  Synthetic peptide
ENSVAYSNNSIAIPT

SEQ ID NO: 910 - protein, 15 aa, Artificial sequence
  Synthetic peptide
ESIVRFPNITNLCPF

SEQ ID NO: 911 - protein, 15 aa, Artificial sequence
  Synthetic peptide
FGGFNFSQILPDPSK

SEQ ID NO: 912 - protein, 15 aa, Artificial sequence
  Synthetic peptide
FPREGVFVSNGTHWF

SEQ ID NO: 913 - protein, 15 aa, Artificial sequence
  Synthetic peptide
GGFNFSQILPDPSKP

SEQ ID NO: 914 - protein, 15 aa, Artificial sequence
  Synthetic peptide
HTPINLVRDLPQGFS

SEQ ID NO: 915 - protein, 15 aa, Artificial sequence
  Synthetic peptide
IGINITRFQTLLALH

SEQ ID NO: 916 - protein, 15 aa, Artificial sequence
  Synthetic peptide
IIAYTMSLGAENSVA

SEQ ID NO: 917 - protein, 15 aa, Artificial sequence
  Synthetic peptide
INITRFQTLLALHRS

SEQ ID NO: 918 - protein, 15 aa, Artificial sequence
  Synthetic peptide
INLVRDLPQGFSALE

SEQ ID NO: 919 - protein, 15 aa, Artificial sequence
  Synthetic peptide
KHTPINLVRDLPQGF

SEQ ID NO: 920 - protein, 15 aa, Artificial sequence
  Synthetic peptide
KLIANQFNSAIGKIQ

SEQ ID NO: 921 - protein, 15 aa, Artificial sequence
  Synthetic peptide
KLIANQFNSVIGKIQ

SEQ ID NO: 922 - protein, 15 aa, Artificial sequence
  Synthetic peptide
KTQSLLIVNNATNVV

SEQ ID NO: 923 - protein, 15 aa, Artificial sequence
  Synthetic peptide
KVEAEVQIDRLITGR

SEQ ID NO: 924 - protein, 15 aa, Artificial sequence
  Synthetic peptide
LHSTQDLFLPFFSNV

SEQ ID NO: 925 - protein, 15 aa, Artificial sequence
  Synthetic peptide
LNTLVKQLSSNFGAI

SEQ ID NO: 926 - protein, 15 aa, Artificial sequence
  Synthetic peptide
LNTLVKQLSSNSGAI

SEQ ID NO: 927 - protein, 15 aa, Artificial sequence
  Synthetic peptide
LYENQKLIANQFNSV

SEQ ID NO: 928 - protein, 15 aa, Artificial sequence
  Synthetic peptide
MAYRFNGIGVTQNVL

SEQ ID NO: 929 - protein, 15 aa, Artificial sequence
  Synthetic peptide
NGLTVLPPLLTDEMI

SEQ ID NO: 930 - protein, 15 aa, Artificial sequence
  Synthetic peptide
NSVAYSNNSIAIPTN

SEQ ID NO: 931 - protein, 15 aa, Artificial sequence
  Synthetic peptide
PIGINITRFQTLLAL

SEQ ID NO: 932 - protein, 15 aa, Artificial sequence
  Synthetic peptide
PINLVRDLPQGFSAL

SEQ ID NO: 933 - protein, 15 aa, Artificial sequence
  Synthetic peptide
PRTFLLKYNENGTIT

SEQ ID NO: 934 - protein, 15 aa, Artificial sequence
  Synthetic peptide
PTESIVRFPNITNLC

SEQ ID NO: 935 - protein, 15 aa, Artificial sequence
  Synthetic peptide
QALNTLVKQLSSNFG

SEQ ID NO: 936 - protein, 15 aa, Artificial sequence
  Synthetic peptide
QALNTLVKQLSSNSG

SEQ ID NO: 937 - protein, 15 aa, Artificial sequence
  Synthetic peptide
QDLFLPFFSNVTWFH

SEQ ID NO: 938 - protein, 15 aa, Artificial sequence
  Synthetic peptide
QKLIANQFNSVIGKI

SEQ ID NO: 939 - protein, 15 aa, Artificial sequence
  Synthetic peptide
QSLLIVNNATNVVIK

SEQ ID NO: 940 - protein, 15 aa, Artificial sequence
  Synthetic peptide
RFQTLLALHRSYLTP

SEQ ID NO: 941 - protein, 15 aa, Artificial sequence
  Synthetic peptide
SIIAYTMSLGAENSV

SEQ ID NO: 942 - protein, 15 aa, Artificial sequence
  Synthetic peptide
SIVRFPNITNLCPFG

SEQ ID NO: 943 - protein, 15 aa, Artificial sequence
  Synthetic peptide
SKTQSLLIVNNATNV

SEQ ID NO: 944 - protein, 15 aa, Artificial sequence
  Synthetic peptide
SVAYSNNSIAIPTNF

SEQ ID NO: 945 - protein, 15 aa, Artificial sequence
  Synthetic peptide
TESIVRFPNITNLCP

SEQ ID NO: 946 - protein, 15 aa, Artificial sequence
  Synthetic peptide
TLVKQLSSNSGAISS

SEQ ID NO: 947 - protein, 15 aa, Artificial sequence
  Synthetic peptide
TPINLVRDLPQGFSA

SEQ ID NO: 948 - protein, 15 aa, Artificial sequence
  Synthetic peptide
TQDLFLPFFSNVTWF

SEQ ID NO: 949 - protein, 15 aa, Artificial sequence
  Synthetic peptide
TQSLLIVNNATNVVI

SEQ ID NO: 950 - protein, 15 aa, Artificial sequence
  Synthetic peptide
VEAEVQIDRLITGRL

SEQ ID NO: 951 - protein, 15 aa, Artificial sequence
  Synthetic peptide
CVNFNFNGLTGTGVL

SEQ ID NO: 952 - protein, 15 aa, Artificial sequence
  Synthetic peptide
FNFNGLTGTGVLTES

SEQ ID NO: 953 - protein, 15 aa, Artificial sequence
  Synthetic peptide
FNGLTLLPPLLTDEM

SEQ ID NO: 954 - protein, 15 aa, Artificial sequence
  Synthetic peptide
FNGLTVLPPLLTDEM

SEQ ID NO: 955 - protein, 15 aa, Artificial sequence
  Synthetic peptide
FNGLTVMPPLLTDEM

SEQ ID NO: 956 - protein, 15 aa, Artificial sequence
  Synthetic peptide
ITSGWTFGAGAALQI

SEQ ID NO: 957 - protein, 15 aa, Artificial sequence
  Synthetic peptide
KCVNFNFNGLTGTGV

SEQ ID NO: 958 - protein, 15 aa, Artificial sequence
  Synthetic peptide
NFNFNGLTGTGVLTE

SEQ ID NO: 959 - protein, 15 aa, Artificial sequence
  Synthetic peptide
NGLTLLPPLLTDEMI

SEQ ID NO: 960 - protein, 15 aa, Artificial sequence
  Synthetic peptide
QPTESIVRFPNITNL

SEQ ID NO: 961 - protein, 15 aa, Artificial sequence
  Synthetic peptide
RTFLLKYNENGTITD

SEQ ID NO: 962 - protein, 15 aa, Artificial sequence
  Synthetic peptide
SGWTFGAGAALQIPF

SEQ ID NO: 963 - protein, 15 aa, Artificial sequence
  Synthetic peptide
TFLLKYNENGTITDA

SEQ ID NO: 964 - protein, 15 aa, Artificial sequence
  Synthetic peptide
TITSGWTFGAGAALQ

SEQ ID NO: 965 - protein, 15 aa, Artificial sequence
  Synthetic peptide
TSGWTFGAGAALQIP

SEQ ID NO: 966 - protein, 15 aa, Artificial sequence
  Synthetic peptide
VNFNFNGLTGTGVLT

(19) United States
(12) Patent Application Publication
(10) Pub. No.: US 2024/0009299 A1
(43) Pub. Date: Jan. 11, 2024

(54) MULTI-EPITOPE VACCINE

(71) Applicant: The Cleveland Clinic Foundation, Cleveland, OH (US)

(72) Inventors: Feixiong Cheng, Cleveland, OH (US); William Martin, Cleveland, OH (US)

(21) Appl. No.: 18/040,531

(22) PCT Filed: Aug. 4, 2021
(86) PCT No.: PCT/US21/44460

§ 371 Date: Feb. 3, 2023

Related U.S. Application Data
(60) Provisional application No. 63/061,404, filed on Aug. 5, 2020.

(51) Int. Cl. A61K 39/215; A61P 31/14

(52) U.S. Cl. CPC A61K 39/215; A61P 31/14; A61K 2039/55516

(57) ABSTRACT

The present invention provides compositions comprising polypeptides comprising a plurality of epitopes from the spike glycoprotein of SARS-CoV-2, systems, and methods of using thereof for the treatment of viral infections (e.g., coronavirus disease 2019 (COVID-19)).

### CROSS-REFERENCE TO RELATED APPLICATIONS

This application claims the benefit of U.S. Provisional Application No. 63/061,404, filed Aug. 5, 2020, the content of which is herein incorporated by reference in its entirety.

### STATEMENT REGARDING FEDERAL FUNDING

This invention was made with government support under HL138272 and AG066707 awarded by the National Institutes of Health. The government has certain rights in the invention.

### SEQUENCE LISTING STATEMENT

The text of the computer readable sequence listing filed herewith, titled “38679-601_SEQUENCE_LISTING_ST25”, created Aug. 4, 2021, having a file size of 281,202 bytes, is hereby incorporated by reference in its entirety.

### FIELD

The present invention provides compositions comprising one or more polypeptides having epitopes from the spike glycoprotein of SARS-CoV-2, systems, and methods of using thereof.

### BACKGROUND

Coronavirus disease 2019 (COVID-19) is an infectious disease caused by severe acute respiratory syndrome coronavirus 2 (SARS-CoV-2). It was first identified in December 2019 in Wuhan, China, and proceeded to spread globally, resulting in an ongoing pandemic. As of the end of June 2020, over 10 million cases of COVID-19 were confirmed in over 200 countries, with complications of COVID-19 cited as the cause of death in over 500,000 individuals.

Common symptoms include fever, cough, fatigue, shortness of breath, and loss of smell and taste. While the majority of cases result in mild symptoms, some progress to an unusual form of acute respiratory distress syndrome (ARDS) likely precipitated by cytokine storm, multi-organ failure, septic shock, vascular inflammation, and blood clots. The time from exposure to onset of symptoms is typically around five days but may range from two to fourteen days. The virus is primarily spread between people during close contact, most often via small droplets produced by coughing, sneezing, and talking. According to the World Health Organization, there are no available vaccines nor specific antiviral treatments for COVID-19.

### SUMMARY

Disclosed herein are polypeptides, compositions, vaccine, and methods for treating a subject with a viral infection (e.g., coronavirus infection).

Disclosed herein is a composition comprising one or more polypeptides comprising a plurality of epitopes from the spike glycoprotein of SARS-CoV-2, wherein the plurality of epitopes comprises at least one of each of: a linear B lymphocyte (LBL) epitope; a cytotoxic T lymphocyte (CTL) epitope; and a helper T lymphocyte (HTL) epitope. In some embodiments, at least a portion of the plurality of epitopes are non-contiguous epitopes from the spike glycoprotein of SARS-CoV-2.

The one or more polypeptides may comprise between one and five (e.g., 1, 2, 3, 4, or 5). LBL epitopes. In some embodiments, the one or more polypeptides comprise five LBL epitopes. In some embodiments, each LBL epitope comprises an amino acid sequence selected from the group consisting of: SEQ ID NOs: 1-5 and 24-446. In certain embodiments, each LBL epitope comprises an amino acid sequence selected from the group consisting of: SEQ ID NOs: 1-5.

The one or more polypeptides may comprise between one and six (e.g., 1, 2, 3, 4, 5, or 6) HTL epitopes. In some embodiments, the one or more polypeptides comprise six HTL epitopes. In some embodiments, each HTL epitope comprises an amino acid sequence selected from the list consisting of: SEQ ID NOs: 6-11 and 447-700. In certain embodiments, each HTL epitope comprises an amino acid sequence selected from the list consisting of: SEQ ID NOs: 6-11.

The one or more polypeptides may comprise between one and six CTL epitopes. In some embodiments, the one or more polypeptides comprise six CTL epitopes. In some embodiments, each CTL epitope comprises an amino acid sequence selected from the list consisting of: SEQ ID NOs: 12-17 and 701-966. In some embodiments, each CTL epitope comprises an amino acid sequence selected from the list consisting of: SEQ ID NOs: 12-17.

In some embodiments, the one or more polypeptides comprise overlapping, partially overlapping, and non-overlapping epitopes. The one or more polypeptides may further comprise linkers between non-overlapping epitopes. In some embodiments, the linker comprises an amino acid sequence of AAY, KK, or GPGPG (SEQ ID NO: 20).

The composition may further comprise an adjuvant. In some embodiments, the adjuvant comprises a peptide adjuvant. In some embodiments, the adjuvant comprises 50S ribosomal L7/L12 protein. In some embodiments, the adjuvant is conjugated to the one or more polypeptides (e.g., at the N-terminus) with a linker. In certain embodiments, the linker comprises an amino acid sequence of SEQ ID NO: 19.

In some embodiments, the composition comprises one polypeptide comprising the plurality of epitopes. In some embodiments, the one polypeptide comprises or consists of an amino acid sequence with at least 70% similarity (e.g., 70% . . . 80% . . . 90% . . . 95% . . . or 99% sequence identity) to SEQ ID NO: 21, SEQ ID NO: 22 or SEQ ID NO: 23.

In some embodiments, the composition further comprises at least one carrier. In certain embodiments the carrier comprises a physiological tolerable buffer.

Also disclosed are methods for reducing or preventing a viral infection in a subject in need thereof or inducing an immune response in a subject. The methods comprise administering to the subject an effective amount of the composition disclosed herein. The administration may comprise an initial immunization and at least one subsequent immunization. In some embodiments, the viral infection comprises a coronavirus infection. In some embodiments, the coronavirus is severe acute respiratory syndrome coronavirus-2 (SARS-CoV-2). In some embodiments, the subject is human.

Further disclosed are nucleic acids, including expression vectors, encoding a polypeptides comprising a plurality of epitopes from the spike glycoprotein of SARS-CoV-2.

The disclosure also provides systems comprising the composition disclosed herein and a delivery device of a container. In some embodiments, the delivery device comprises a syringe. In some embodiments, the composition is pre-loaded in the delivery device. In some embodiments, the contain comprises a syringe vial. The composition may be in the syringe vial. The system may further comprise a packaging component. In some embodiments, the packaging component contains the container and the composition is inside the container.

Other aspects and embodiments of the disclosure will be apparent in light of the following detailed description and accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic of the overall workflow used during development of some of the embodiments described herein: i) selection of the systems used to generate conformations to be used in the linear B lymphocyte prediction; ii) epitope predictions, including linear B lymphocyte, cytotoxic T lymphocyte, and helper T lymphocytes—predicted epitopes were assessed for multiple immune-relevant properties, as well as their ability to be accessed by a simulated antibody (antibody accessible surface area, AbASA); iii) final selected epitopes were linked together, along with an N-terminal adjuvant, using linkers—sequence was assessed for immune-relevant properties and simulated immune response; and iv) secondary and tertiary structures were predicted and refined, and the final 3D structure was docked using protein-protein docking to toll-like receptor 2 and 4 (TLR2/4).

FIG. 2 shows antibody-accessible surface area (AbASA) of the spike glycoprotein. In the left image, regions of the spike glycoprotein which have at least 0.25 Å² of AbASA are shown in blue, regions with less AbASA are shown in red, and the glycosylation is shown in gray. Percent change in AbASA due to glycosylation (right panel) was shown as blue for no change, green for a 50% reduction in AbASA, and yellow for a 75% reduction in AbASA. A value of 100% (red) was assigned for any residue with less than 0.25 Å² of AbASA.

FIG. 3 shows a schematic representation of the final multi-epitope vaccine construct (left side) and the location of the selected epitopes on the spike glycoprotein (right side). White residues indicate they are not in the final multi-epitope vaccine construct, with glycosylation in gray. Epitopes are labeled as in Table 1.

FIG. 4 is a graph of cytokine levels induced by repeated injection of the vaccine construct. Levels were modeled in C-ImmSim based on three injections given 4 weeks apart. D in the inset plot is the danger signal (dotted line).

FIGS. 5A-5D show the construction and refinement of the multi-epitope vaccine construct. FIG. 5A is a final 3D model of the vaccine construct after modeling with I-TASSER. FIG. 5B is a refined model after refinement with ModRefiner and GalaxyRefine. FIG. 5C shows the structure validation with ProSA-web, indicating the structural properties are in line with other proteins of similar size (Z-score −7.41). FIG. 5D is a Ramachandran plot indicating 92.7% of residues are in favored regions, and 2 residues are in outlier regions.

FIGS. 6A-6D are the protein-protein docking results of adjuvant or vaccine construct with TLR2 or TLR4. Results are from HADDOCK (High Ambiguity Driven protein-protein DOCKing) for the adjuvant and TLR2 (FIG. 6A), adjuvant and TLR4 (FIG. 6B), vaccine and TLR2 (FIG. 6C), and vaccine and TLR4 (FIG. 6D).

FIG. 7 is a schematic of the in silico cloning of the vaccine construct using the pET30a (+) expression vector. The vaccine insertion is denoted with a gray bar. The His-tag is located at the C-terminal end. MCS: multiple cloning site; KanR: kanamycin resistance protein.

FIGS. 8A-8I are graphs of the immune simulation results from C-ImmSim. Injections were simulated to occur at T=0, 4, and 8 weeks. Antigen and immunoglobulin levels, subdivided per isotype (FIG. 8A); B lymphocytes by total count (FIG. 8B) and population per entity state (active, presenting, internalized, duplicating, or anergic) (FIG. 8C); CD4 T-helper lymphocytes by count (FIG. 8D) and population per entity state (FIG. 8E); CD8 T-cytotoxic lymphocytes by count (FIG. 8F) and population per entity state (FIG. 8G); total count of macrophages (FIG. 8H) and dendritic cells (FIG. 8I).

FIG. 9 is a root-mean squared deviation plot over 500 nanoseconds of molecular dynamics simulation for all systems assessed for B-cell epitopes.

FIG. 10 is a graph of the antibody-accessible surface area for the spike glycoprotein when the glycosylation is removed.

FIG. 11 is a graph of the antibody-accessible surface area for the spike glycoprotein when the glycosylation is taken into account. Surface area for the glycans is not included for clarity.

FIG. 12 is a representation of the predicted secondary structure for a multi-epitope vaccine construct (SEQ ID NO: 21). PSIPRED predicted a protein with secondary structure composed of 42.6% alpha helix, 9.4% beta sheet, and 48.0% coil.

FIG. 13 is a graph of the predicted disordered residue profile. 50 of the 331 residues (17%) were predicted to be disordered by RaptorX Property.

### DETAILED DESCRIPTION

Described herein are compositions comprising multi-epitope polypeptides comprising epitopes across linear B lymphocytes (LBL), cytotoxic T lymphocytes (CTL) and helper T lymphocytes (HTL) derived from both mutant and wild-type spike glycoproteins from SARS-CoV-2 with diverse protein conformations. In some embodiments, the polypeptide (35.9 kDa), referred to as COVCCF, comprises 5 LBL, 6 HTL, and 6 CTL epitopes from the spike glycoprotein of SARS-CoV-2. COVCCF induced elevated levels of immunoglobulin activity (IgM, IgG1, IgG2), induced strong responses from B lymphocytes, CD4 T-helper lymphocytes, and CD8 T-cytotoxic lymphocytes, and induced cytokines important to innate immunity, including IFN-γ, IL4, and IL10. Additionally, COVCCF has ideal pharmacokinetic properties and low immune-related toxicities.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “epitope,” as used herein, refers to antigenic peptide fragments, typically derived from a pathogen protein, that when presented by a major histocompatibility complex (MHC) molecule, interact with specific cell receptors (e.g. B cells or T cells) after transport to the surface of an antigen-presenting cell. In some embodiments, the epitopes may be linear or continuous, such that the epitopes correspond to a contiguous amino acid sequence or peptide fragment. In some embodiments, the epitopes are conformational or discontinuous epitopes, such that the epitope contains amino acids that are not contiguous in the sequence of the peptide fragment but are brought into close proximity within the entirety of the folded protein structure.

As used herein, the term “linker” refers to a short polypeptide sequence interposed between any two non-overlapping epitopes or a terminal epitope and an adjuvant. The linker is preferably a polypeptide linker of 1-10, e.g., 2, 3, 4, or 6 amino acids.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide,” “polypeptide” or “protein” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The “peptide,” “polypeptide” or “protein” may be modified by the addition of sugars, lipids or other moieties not included in the amino acid chain. Chains of less than ten or fifteen amino acids are generally referred to oligopeptides, whereas chains of greater than about fifteen amino acids are generally referred to polypeptides or proteins. The terms “polypeptide” and “protein,” are used interchangeably herein.

The term “vaccine,” as used herein, refers to any pharmaceutical composition containing at least one immunogen, which composition can be used to prevent or treat a disease or condition in a subject.

As used herein, “treat,” “treating,” and the like means a slowing, stopping, or reversing of progression of a disease or disorder when provided in a composition described herein to an appropriate subject. The term also includes a reversing of the progression of such a disease or disorder to a point of eliminating or greatly reducing the disease. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease.

As used herein, the terms “providing”, “administering,” and “introducing,” are used interchangeably herein and refer to the placement of the active agents or compositions of the disclosure into a subject by a method or route which results in at least partial localization of the composition to a desired site. The compositions can be administered by any appropriate route which results in delivery to a desired location in the subject.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment, the mammal is a human.

### 2. Compositions

Disclosed herein are compositions comprising one or more polypeptides comprising a plurality of epitopes from the spike glycoprotein of SARS-CoV-2. The plurality of epitopes comprises at least one of each of: a linear B lymphocyte (LBL) epitope; a cytotoxic T lymphocyte (CTL) epitope; and a helper T lymphocyte (HTL) epitope. In some embodiments, at least a portion of the epitopes are non-contiguous.

The one or more polypeptides may comprise between one and five LBL epitopes (e.g., 1, 2, 3, 4, or 5). In some embodiments, the one or more polypeptides comprise five LBL epitopes. The LBL epitopes may each comprise an amino acid sequence selected from the group consisting of SEQ ID NOs: 1-5 and 24-446. In some embodiments, the LBL epitopes may each comprise an amino acid sequence selected from the group consisting of SEQ ID NOs: 1-5.

The one or more polypeptides may comprise between one and six HTL epitopes (e.g., 1, 2, 3, 4, 5, or 6). In some embodiments, the one or more polypeptides comprise six HTL epitopes. The HTL epitopes may each comprise an amino acid sequence selected from the group consisting of SEQ ID NOs: 6-11 and 447-700. In some embodiments, the HTL epitopes may each comprise an amino acid sequence selected from the group consisting of SEQ ID NOs: 6-11 and 447-700.

The one or more polypeptides may comprise between one and six CTL epitopes (e.g., 1, 2, 3, 4, 5, or 6). In some embodiments, the one or more polypeptides comprise six CTL epitopes. The CTL epitopes may each comprise an amino acid sequence selected from the group consisting of SEQ ID NOs: 12-17 and 701-966. In some embodiments, CTL epitopes may each comprise an amino acid sequence selected from the group consisting of SEQ ID NOs: 12-17.

The plurality of epitopes may be fully overlapping, such that an entire epitope is encompassed in one of the other epitopes (e.g., a CTL or HTL epitope may be encompassed in an LBL epitope). The plurality of epitopes may be partially overlapping, such that the C-terminal residues of an epitope correspond to the N-terminal residues of another epitope. In some embodiments, the epitopes may be non-overlapping or have no sequence similarity with another epitope.

The one or more polypeptides may further comprise a linker between non-overlapping epitopes. In some embodiments, the linker comprises an amino acid sequence of AAY. In some embodiments, the linker comprises an amino acid sequence of KK. In some embodiments, the linker comprises an amino acid sequence of GPGPG (SEQ ID NO: 20).

The compositions described herein may be used to prepare vaccines. Vaccine preparation is a well-developed art and general guidance in the preparation and formulation of vaccines is readily available from any of a variety of sources. For example, New Trends and Developments in Vaccines, edited by Voller et al., University Park Press, Baltimore, Md., U.S.A. 1978 and Powell and Newman, eds., Vaccine Design (the subunit and adjuvant approach), Plenum Press (NY, 1995), incorporated herein by reference.

In some embodiments, the composition further comprises an adjuvant or immunostimulant. Adjuvants and immunostimulants are compounds that either directly or indirectly stimulate the immune system's response to a co-administered vaccine or antigen. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); mineral salts (for example, aluminum, silica, kaolin, and carbon); aluminum salts such as aluminum hydroxide gel (alum), AlK(SO₄)₂, AlNa(SO₄)₂, AlNH₄(SO₄), and Al(OH)₃; salts of calcium (e.g., Ca₃(PO₄)₂), iron or zinc; an insoluble suspension of acylated tyrosine; acylated sugars; cationically or anionically derivatized polysaccharides; polynucleotides (for example, poly IC and poly AU acids); polyphosphazenes; cyanoacrylates; polymerase-(DL-lactide-co-glycoside); biodegradable microspheres; liposomes; lipid A and its derivatives; monophosphoryl lipid A; wax D from Mycobacterium tuberculosis, as well as substances found in Corynebacterium parvum, Bordetella pertussis, and members of the genus Brucella); bovine serum albumin; diphtheria toxoid; tetanus toxoid; edestin; keyhole-limpet hemocyanin; Pseudomonal Toxin A; choleragenoid; cholera toxin; pertussis toxin; viral proteins; and Quil A Aminoalkyl glucosamine phosphate compounds can also be used (see, e.g., WO 98/50399, U.S. Pat. No. 6,113,918 (which issued from U.S. Ser. No. 08/853,826), and U.S. Ser. No. 09/074,720). In addition, adjuvants such as cytokines (e.g., GM-CSF or interleukin-2, -7, or -12), interferons, or tumor necrosis factor, may also be used as adjuvants. Protein and polypeptide adjuvants may be obtained from natural or recombinant sources according to methods well known to those skilled in the art. When obtained from recombinant sources, the adjuvant may comprise a protein fragment comprising at least the immunostimulatory portion of the molecule. Other known immunostimulatory macromolecules which can be used include, but are not limited to, polysaccharides, tRNA, non-metabolizable synthetic polymers such as polyvinylamine, polymethacrylic acid, polyvinylpyrrolidone, mixed polycondensates (with relatively high molecular weight) of 4′,4-diaminodiphenylmethane-3,3′-dicarboxylic acid and 4-nitro-2-aminobenzoic acid (See, Sela, M., Science 166: 1365-1374 (1969)) or glycolipids, lipids or carbohydrates.

In some embodiments, the adjuvant comprises a protein or peptide adjuvant. Protein and peptide adjuvants may be obtained from natural or recombinant sources according to methods well known to those skilled in the art. The peptide adjuvant may be synthetic and designed to mimic natural adjuvants. When obtained from recombinant sources, the adjuvant may comprise a protein fragment comprising at least the immunostimulatory portion of the molecule. The adjuvant polypeptide can be any peptide adjuvant known in art including, but not limited to, flagellin, human papillomavirus (HPV) L1 or L2 proteins, herpes simplex glycoprotein D (gD), complement C4 binding protein, synthetic and natural peptide TLR agonists (e.g., toll-like receptor-4 (TLR4) ligand), IL-1(3, and immunomodulating peptides (e.g., defensins, LL37). In some embodiments, the adjuvant comprises the 50S ribosomal L7/L12 protein. In some embodiments, the amino acid sequence of the 50S ribosomal L7/L12 protein comprises SEQ ID NO: 18.

SEQ ID NO: 18
MAKLSTDELLDAFKEMTLLELSDFVKKFEETFEVTAAAPV
AVAAAGAAPAGAAVEAAEEQSEFDVILEAAGDKKIGVIKV
VREIVSGLGLKEAKDLVDGAPKPLLEKVAKEAADEAKAKL
EAAGATVTVK

The adjuvant may be conjugated to the N- or to the C-terminal end of the one or more polypeptides of the composition. In some embodiments, the adjuvant is conjugated to the N-terminus of the one or more polypeptides. In the case of a peptide adjuvant, the N-terminus of the one or more polypeptides may be conjugated to the C-terminus of the peptide adjuvant. In some embodiments, the adjuvant is conjugated to the one or more polypeptides with a linker. The linker may comprise any flexible linker, including by not limited to, glycine rich linkers, serine-rich linkers, or the like. In some embodiments, the linker comprises and amino acid sequence EAAAK (SEQ ID NO: 19). In instances in which the one or more polypeptides comprise an adjuvant, an additional adjuvant may or may not be included in the composition or vaccine.

The composition may comprise any number of polypeptides encoding the plurality of epitopes. The epitopes may be arranged in any order within the one or more polypeptides. In some embodiments, the composition comprises one polypeptide comprising the plurality of epitopes (e.g., non-contiguous epitopes). In some embodiments, the composition comprises one polypeptide comprising amino acid sequences of SEQ ID NOs: 1-17.

In some embodiments, the polypeptide comprises an amino acid sequence with at least 70% similarity to SEQ ID NO: 21, SEQ ID NO: 22 or SEQ ID NO: 23. The polypeptide may comprise an amino acid sequence with at least 70%, at least 75%, at least 80%, at least 85%, at least 90%, at least 95%, or at least 98% similarity to SEQ ID NO: 21, SEQ ID NO: 22 or SEQ ID NO: 23.

SEQ ID NO: 21
MAKLSTDELLDAFKEMTLLELSDFVKKFEETFEVTAAAPV
AVAAAGAAPAGAAVEAAEEQSEFDVILEAAGDKKIGVIKV
VREIVSGLGLKEAKDLVDGAPKPLLEKVAKEAADEAKAKL
EAAGATVTVKEAAAKFEYVSQPFLMDLEGKGPGPGFNATR
FASVYAWNRKAAYSPRRARSVAKKNSNNLDSKVGGNYNYL
YKKIYSKHTPINLVRDLPQGFSALEPLVDLPIGKKLPFFS
NVTWFHAIHVSGTNGTKRFDNPVLPFKKTPCNGVEGENCY
FPLQSYGFQPTNGKKFQTLLALHRSYLTPGDSSSGWTAGA
AAYYVHHHHHH
SEQ ID NO: 22
MAKLSTDELLDAFKEMTLLELSDFVKKFEETFEVTAAAPV
AVAAAGAAPAGAAVEAAEEQSEFDVILEAAGDKKIGVIKV
VREIVSGLGLKEAKDLVDGAPKPLLEKVAKEAADEAKAKL
EAAGATVTVKEAAAKFEYVSQPFLMDLEGKGPGPGFNATR
FASVYAWNRKAAYSPRRARSVAKKNSNNLDSKVGGNYNYL
YKKIYSKHTPINLVRDLPQGFSALEPLVDLPIGKKLPFFS
NVTWFHAIHVSGTNGTKRFDNPVLPFKKTPCNGVEGENCY
FPLQSYGFQPTNGKKFQTLLALHRSYLTPGDSSSGWTAGA
AAYYV
SEQ ID NO: 23
FEYVSQPFLMDLEGKGPGPGFNATRFASVYAWNRKAAYSP
RRARSVAKKNSNNLDSKVGGNYNYLYKKIYSKHTPINLVR
DLPQGFSALEPLVDLPIGKKLPFFSNVTWFHAIHVSGTNG
TKRFDNPVLPFKKTPCNGVEGFNCYFPLQSYGFQPTNGKK
FQTLLALHRSYLTPGDSSSGWTAGAAAYYV

The compositions may further comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, coatings, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, coating agents, sweetening, flavoring and perfuming agents, preservatives and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

The compositions may be formulated for any appropriate manner of administration, and thus administered, including for example, topical, oral, nasal, intravenous, intravaginal, epicutaneous, sublingual, intracranial, intradermal, intraperitoneal, subcutaneous, intramuscular administration, or via inhalation. Techniques and formulations may generally be found in “Remington's Pharmaceutical Sciences,” (Meade Publishing Co., Easton, Pa.). Therapeutic compositions must typically be sterile and stable under the conditions of manufacture and storage. The route or administration and the form of the composition will dictate the type of carrier to be used.

The compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, solutes that render the formulation isotonic, hypotonic or weakly hypertonic with the blood of a recipient, suspending agents, thickening agents and/or preservatives, commonly found in vaccine compositions.

The compositions may also contain other compounds, which may be biologically active or inactive. For example, one or more immunogenic portions of other antigens may be present in any known form. The compositions may generally be used for prophylactic and therapeutic purposes.

The present disclosure also provides nucleic acids encoding polypeptides comprising a plurality of non-contiguous epitopes from the spike glycoprotein of SARS-CoV-2. The description provided above for the polypeptides and epitopes is relevant to the nucleic acids disclosed here. In some embodiments, the nucleic acids disclosed herein can be introduced into an expression vector, such that the expression vector comprises a promoter and the nucleic acids encoding the polypeptides described herein. The expression vector may allow expression of the polypeptide in a suitable expression system using techniques well known in the art, followed by isolation or purification of the expressed polypeptide of interest. A variety of bacterial, yeast, plant, mammalian, and insect expression systems are available in the art and any such expression system can be used. Alternatively, a nucleic acids encoding a peptide of the invention can be translated in a cell-free translation system.

### 3. Methods of Use

The present disclosure provides methods for reducing or preventing a viral infection in a subject in need thereof. The disclosure also provides methods of inducing an immune response in a subject. The methods include administering to the subject an effective amount of the compositions, disclosed herein. An “effective amount” of the compositions, is an amount that is delivered to a subject, either in a single dose or as part of a series, which is effective for inducing an immune response against the viral infection in the subject. The viral infection may be a coronavirus infection. In some embodiments, the coronavirus is severe acute respiratory syndrome coronavirus-2 (SARS-CoV-2).

The compositions disclosed herein can be administered in a wide variety of therapeutic dosage forms in the conventional vehicles for topical, oral, systemic, local, and parenteral administration.

The route and regimen of administration will vary depending upon the population and the indication for vaccination and is to be determined by the skilled practitioner. For example, the compositions disclosed herein may be administered in such oral dosage forms for example as tablets, capsules (each including timed release and sustained release formulations), pills, powders, granules, elixirs, tinctures, solutions, suspensions, syrups and emulsions, or by injection. Similarly, they may also be administered parentally, e.g., in intravenous (either by bolus or infusion methods), intraperitoneal, subcutaneous, topical with or without occlusion, or intramuscular form.

The administration may comprise an initial immunization or dose and at least one subsequent immunization or booster dose, following known standard immunization protocols. The boosting doses will be adequately spaced at such times where the levels of circulating antibody fall below a desired level. Boosting may comprise alternative carriers and/or adjuvants. The booster dosage levels may be the same or different that those of the initial immunization dosage.

The specific dose level may depend upon a variety of factors including the activity of the peptide, composition or vaccine, the age, body weight, general health, and diet of the subject, time of administration, and route of administration. For prophylaxis purposes, the amount of polypeptide in each dose is an amount which induces an immunoprotective response without significant adverse side effects.

The compositions may be prepared, packaged, or sold in a form suitable for bolus administration or sold in unit dosage forms, such as in ampules or multi-dose containers containing a preservative. Also disclosed herein is a system comprising the compositions disclosed herein and a delivery device or container. In some embodiments, the delivery device or container comprises a syringe or syringe vial. In some embodiments, the delivery device or container is pre-filled with the composition.

### 4. Examples

### Materials and Methods

System Selection Prior to epitope prediction, a total of 10 systems were simulated using the GROMACS 2020.1¹¹ molecular dynamics package, including 9 mutated systems and wild type. The selected mutants (A852V, A930V, F797C, F970S, L752F, L861M, V615L, V860D, V860L) were selected³² based on conservation of the residue between SARS-CoV-2, SARS-CoV-1, and MERS-CoV. Each of the 9 mutations were added in silico using CHARMM-GUI^{33,34} using PDB ID 6VSB35 at the time of system creation. Following a processing step involving the addition of hydrogen atoms and completion of missing loops, a water box with edges at least 10 angstroms from any part of the protein was added. The systems were neutralized and brought to a total ionic strength of 0.15 M using sodium and chloride ions. Parameterization of the protein, ions, and TIP3P water molecules was accomplished using the CHARMM36m force field³⁶. Each of these systems used the glycosylation state as in the crystal structure with no modifications. An additional wild type system with high mannose N-glycans was constructed in order to assess the change in the proteins immune accessibility due to the glycan shield.

Simulation Parameters All systems were simulated using GROMACS 2020.1 on the AiMOS Supercomputer at the Rensselaer Polytechnic Institute Center for Computational Innovations in a three-step process. Initial minimization of the systems was run until changes in the potential energy of the system reached machine precision. Following minimization, an NVT equilibration step was completed with a 2 fs timestep for 500,000 steps using 400 kJ mol⁻¹ nm⁻² and 40 kJ mol⁻¹ nm⁻² positional restraints on the backbone and sidechains, respectively. A 500 ns production step was completed using the NPT ensemble with no position restraints and a 2 fs timestep.

Hydrogen atoms were constrained using the LINCS37 algorithm. Temperature for the system was held at 300 K using a Nose-Hoover thermostat³⁸ with a 1 ps coupling constant. For the production simulation, pressure was coupled isotropically using a Parrinello-Rahman barostat³⁹ with a 5.0 ps coupling constant and compressibility of 4.5e⁻⁰⁵ bar⁻¹ to maintain a pressure of 1 bar. The pair-list cutoff was constructed using the Verlet scheme⁴⁰ with a cutoff distance of 1.2 nm. Particle mesh Ewald electrostatics⁴¹ were used to describe coulombic interactions with a 1.2 nm cutoff, while van der Waals forces were smoothly switched to using between 1.0 and 1.2 nm using a force-switch modifier to the cut-off scheme. Linear center of mass translation was removed every 100 steps for the entire system.

Antibody-Accessible Surface Area Determination To determine which predicted epitopes are most likely to be capable of eliciting a useful immune response, antibody-accessible surface area (AbASA) was determined the using a method similar to that outlined previously (Grant, 0. C., et al., bioRxiv 2020.04.07.030445 (2020), incorporated herein by reference in its entirety). Two calculations for AbASA were completed using the built in SASA tool in GROMACS 2020.1, selecting a probe size of 0.72 nanometers as opposed to the standard 0.14 nanometer probe used for a standard SASA calculation. The first calculation determined the AbASA for the bare protein, not accounting for glycosylation, while the second determined the AbASA for the protein while also taking the extensive glycosylation into account. When selecting an epitope for COVCCF, a residue was deemed to be not antibody accessible if its AbASA was lower than 0.25 Å². As the spike glycoprotein is a homotrimer, an average of the AbASA across the three domains was used for this determination. Additionally, residues with a drop in AbASA when considering the glycan shield were inspected on a case-by-case basis with the knowledge that the 0.72 nm probe radius would only account for accessibility for an average loop in an antibody and did not account for accessibility of an entire antibody. Regions which had a large change in AbASA were determined to be shielded, and predicted epitopes for these regions were not included in COVCCF.

Linear B-cell Epitope Prediction The above simulations were sampled at t=0, 100, 200, 300, 400, and 500 nanoseconds for the prediction of linear B lymphocyte (LBL) epitopes, yielding a total of 60 structures. These structure-based predictions were made using ElliPro¹² using the default minimum score of 0.5 and the default maximum distance of 6 angstroms. ElliPro implements three algorithms in its predictions: 1) an approximation of the shape of the protein as an ellipsoid; 2) calculation of the protrusion index for each residue, which is a quantification of the extent to which a residue protrudes from the surface of a protein based on the ellipsoid approximations; and 3) a clustering of neighboring residues based on protrusion index. While ElliPro is able to predict both linear and conformational epitopes, only linear epitopes are used in vaccine design⁴³. Since only structural epitopes were generated, only residues 27 through 1146 were included in any of the epitope predictions, as those are the only residues crystallized in the pdb used.

Cytotoxic T Lymphocytes (CTL) Epitope Prediction Cytotoxic T lymphocyte (CTL) epitopes were predicted for each of the 10 sequences noted above using the NetCTL 1.2 server⁴⁴. The default threshold for epitopes was retained at 0.75, and all 12 of the available MHC class I supertypes was used for the predictions. NetCTL uses artificial neural networks to predict major histocompatibility class (MHC) I binding and proteasomal cleavage, while TAP transport efficiency is predicted using a weight matrix. Additionally, the CTL epitopes were each evaluated for their immunogenicity using the MHC-1 immunogenicity tool on the IEDB server⁴⁵.

Helper T Lymphocytes (HTL) Epitope Prediction Helper T cells help activate B cells to secrete antibodies and macrophages, and also help activate cytotoxic T cells, indicating their importance to adaptive immunity. Prediction of these HTL epitopes as peptides that bind MHC II molecules is therefore key to rational vaccine design⁴⁶. HTL epitopes of length 15 were predicted using the IEDB MHC-II binding predictions tool. The IEDB recommended prediction method was selected, which uses the consensus approach⁴⁷, combining NN-align⁴⁸, SMM-align⁴⁶, CombLib⁴⁹, and Sturniolo⁵⁰ when possible, otherwise using NetMHCIIpan⁵¹. The full HLA reference set was used for the prediction, and predictions with a percentile rank ≤2 were chosen; a lower percentile rank indicates a higher affinity.

Assessment of CTL/LBL Epitopes for Antigenicity, Allergenicity, and Toxicity To ensure their ability to illicit an immune response, the antigenicity of each of the CTL and LBL epitopes was evaluated using the VaxiJen 2.0 server¹⁵. VaxiJen uses an alignment-free approach based on auto cross covariance (ACC) transformation, a protein sequence mining method which has been applied to quantitative structure-activity relationships (QSAR) studies and protein classification⁵². This application of ACC to the principal component analysis (PCA) of 29 properties of each of the 20 amino acids allows for the removal of irrelevant information, amplifying class-discriminating properties. Allergenicity of epitopes was determined using the AllerTOP 2.0 server¹⁶, which in addition to ACC uses a k-nearest neighbor algorithm based on a training set consisting of 2427 each of known allergens and non-allergens from different species. Toxicity of epitopes was predicted using the ToxinPred⁵³ server, which uses the Support Vector Machine (SVM) algorithm, with a main dataset including 1805 sequences as positive training data and 3593 negative sequences from Swissprot⁵⁴, and an independent dataset comprising of 303 positive and 300 negative sequences, also from Swissprot.

Identification of Cytokine-Inducing HTL epitopes The ability of an HTL epitope to induce cytokines (specifically, interferon-gamma [IFN-γ], interleukin-4 [IL-4], and interleukin-10 [IL-10]) is key to a vaccine's ability to illicit an effective immune response; the release of these cytokines helps in the activation of cytotoxic T-cells and other immune cells¹³. To determine the ability of our predicted HTL epitopes to induce these cytokines, the IFNepitope server¹³ (IFN-γ) using the motif and SVM hybrid approach with the IFN-gamma versus non IFN-gamma model; the IL4pred server⁵⁵ (IL-4) using the hybrid (SVM+motif) and the default SVM threshold of 0.2; and the IL10pred server⁵⁶ (IL-10) using the SVM based method with the default SVM threshold of −0.3 were used. These prediction servers, like ToxinPred above, use the SVM algorithm for their predictions.

Construction of the Multi-Epitope Polypeptide Sequence The CTL, HTL, and LBL epitopes which passed the above tests were used to generate the full vaccine sequence. In the event of overlap between epitopes, the sequence was not duplicated. Epitopes were linked together using GPGPG, AAY, and KK linkers; GPGPG and AAY linkers were used to connect the HTL and CTL epitopes, while KK linkers were used for B-cell epitopes, allowing them to preserve their independent immunogenic properties⁵⁷. A 50 S ribosomal protein L7/L12 (locus RL7_MYCTU, UniProtKB ID: P9WHE3) was chosen as an adjuvant, and inserted on the N-terminus using an EAAAK linker.

Prediction of Physiochemical Properties, Solubility, Allergenicity, Antigenicity, and IFN-γ induction The full sequence for the multi-epitope vaccine construct COVCCF was tested for its allergenicity using the AllerTOP 2.0 server, and its antigenicity using the VaxiJen 2.0 server. The IFNepitope server was used to scan the full sequence for predicted IFN-γ inducing epitopes using the SVM based method for score prediction only. The ProtParam web server⁵⁸ allows for the prediction of various physiochemical properties, including amino acid composition, theoretical isoelectric point (pI), instability index, half-life (both in vivo and in vitro) aliphatic index, molecular weight, and grand average of hydropathicity (GRAVY). Solubility of the final protein sequence was predicted using the CamSol server^{17,59}, which allows for a pdb structure as input, taking into account the 3D conformation of the protein as opposed to only the sequence.

Prediction of Secondary Structure The generated sequence for the full-length vaccine was submitted to the PSIPRED 4.0 server¹⁸ to predict its secondary structure. PSIPRED uses a deep neural network architecture with two hidden layers and rectifier activations; the current version has a Q3 secondary structure prediction accuracy of 84.2%. The RaptorX Property server¹⁹ was additionally employed as a second validation. RaptorX Property employs a new machine learning model, Deep Convolutional Neural Fields (DeepCNF), which implements both conditional neural fields (CNF) and deep convolutional neural networks (DCNN).

Tertiary Structure Prediction Homology modeling of the final multi-epitope polypeptide was completed using the I-TASSER server²⁰. I-TASSER (Iterative Threading ASSEmbly Refinement) uses a three-step process to model the tertiary structure of a protein. First, the server tries to retrieve template proteins from the PDB library using LOMETS (Local Meta-Threading Server), which generates protein structure predictions by ranking and selecting models from multiple state of the art threading programs⁶⁰. The second step involves assembling fragments excised from the PDB templates determined in step 1 using replica-exchange Monte Carlo simulations, with unaligned regions generated using ab initio modeling. The third step integrates spatial restraints to remove steric clashes, finally generating full atomic models. The secondary structure predictions generated by PSIPRED were submitted along with the primary structure of the multi-epitope polypeptide.

Tertiary Structure Refinement. The selected model generated by I-TASSER was further refined first using ModRefiner²¹, followed by GalaxyRefine²². ModRefiner uses a two-step process, first a low-resolution step followed by a high-resolution step. The low-resolution step begins with a C-alpha trace of the initial structure, adding main chain atoms for an energy minimization. This structure is then passed to the high-resolution step, which adds side chain atoms and does a full atomic energy minimization, yielding a final model. GalaxyRefine begins by rebuilding all side chains, placing the highest probability rotamers starting from the core and extending to the surface, layer by layer. Upon reaching a steric clash, the next highest probability rotamer is selected. After being rebuilt, the model is refined using a two-step relaxation process, of which the lowest energy model is returned as model 1, and additional models are returned based on the closest clustered models.

Structural Validation Multiple servers were implemented to validate the tertiary structure generated. ProSAweb²³ gives an assessment of the overall model quality, displayed in the context of all Xray and NMR structures, with a Z-score falling outside that of known structures generally implies errors in the structure. The ERRAT server⁶¹ was used to assess non-bonded interactions in comparison to high-resolution crystallographic structures. Finally, the MolProbity⁶² server was used to generate a Ramachandran plot, which gives a visualization of energetically allowed and disallowed dihedral angles of amino acids, calculated based on the van der Waal radius of the side chain.

Prediction of LBL Epitopes in the Vaccine Protein The presence of both linear and discontinuous B-cell epitopes was predicted using ElliPro as above. It has been estimated that greater than 90% of B-cell epitopes are discontinuous; that is, their segments are distant from each other in their primary structure but are brought close to each other upon the folding of the protein^{63,64}.

In Silico Cloning of Designed Multi-Epitope Vaccine The vaccine protein sequence was submitted to the JAVA Codon Adaptation Tool (JCAT)²⁷ to adapt the codon usage to E. coli K12. The options to avoid rho-independent transcription termination, prokaryote ribosome binding site, and restriction enzyme cleavage sites were selected. XhoI and XbaI restriction sites were added to the C and N termini of the sequence, respectively. The final nucleotide sequence was then cloned into the pET-30a (+) vector using the SnapGene 5.1.3 software.

Molecular Docking of the Vaccine Construct with TLR2/TLR4 The ability for COVCCF to generate an immune response depends on its ability to interact with immune receptors. Toll-like receptors 2 and 4 (TLR2, TLR4) are members of the TLR family, which play a role in pathogen recognition and activation of innate immunity. Therefore, the ability for COVCCF to interact with these receptors is key to the immune response. The adjuvant was selected as the region of interest, as it has been shown to be a TLR agonist²⁶. CPORT²⁵ was used to initially predict residues which could be involved in the protein-protein interaction. The results from this initial prediction were imported into the HADDOCK 2.4 server²⁴ for data-driven protein-protein docking. HADDOCK (High Ambiguity Driven protein-protein DOCKing) uses a collection of python scripts which make use of crystal and NMR structures for structure calculations. The best structure from the best cluster was submitted to the PRODIGY (PROtein binDIng enerGY prediction) server⁶⁵ to predict the binding affinities of each of the protein-protein complexes.

Immune Simulation The immunogenicity of COVCCF was further characterized using the C-ImmSim server⁶⁶. C-ImmSim uses position-specific scoring matrix (PSSM) for immune epitope prediction and machine learning to predict immune interactions. It simulates hematopoietic stem cells in the bone marrow, T-cells in the thymus, and tertiary lymphatic organs, for their immune response. It has been determined computationally that an interval of several weeks between the prime (first) and boost (all subsequent) doses of a vaccine is required to obtain optimal antibody response¹⁴. Therefore, the simulation was set to administer three injections at timesteps 1, 84, and 168, corresponding to time=0, 4 weeks, and 8 weeks with a total of 1050 simulation steps. Each injection contained 1000 vaccine proteins, and all other parameters were set to their defaults. A further simulation with 12 injections setting 4 weeks apart was also carried out, which would simulate repeated exposure as typically seen in an endemic area, probing the clonal selection. The Simpson Index D, a measure of diversity, was interpreted from the plot.

### Example 1

### SARS-CoV-2 Virus-Host Interactome

The spike glycoprotein (S protein)¹ in SARS-CoV-2 interacts with angiotensin-converting enzyme 2 (ACE2) via its receptor binding domain (RBD)². The S protein is a 180 kDa homotrimer consisting of two subunits, S1 and S2, which mediate attachment to ACE2 and membrane fusion, respectively³. The 51 subunit consists of an N-terminal domain (NTD) and the RBD, while the S2 subunit is composed of a fusion protein (FP), two heptad repeat domains (HR1 and HR2), a transmembrane domain (TM), and a cytoplasmic domain (CP)⁴. In order to fuse its viral membrane with the host cell, the S protein is activated at the S1/S2 boundary⁵.

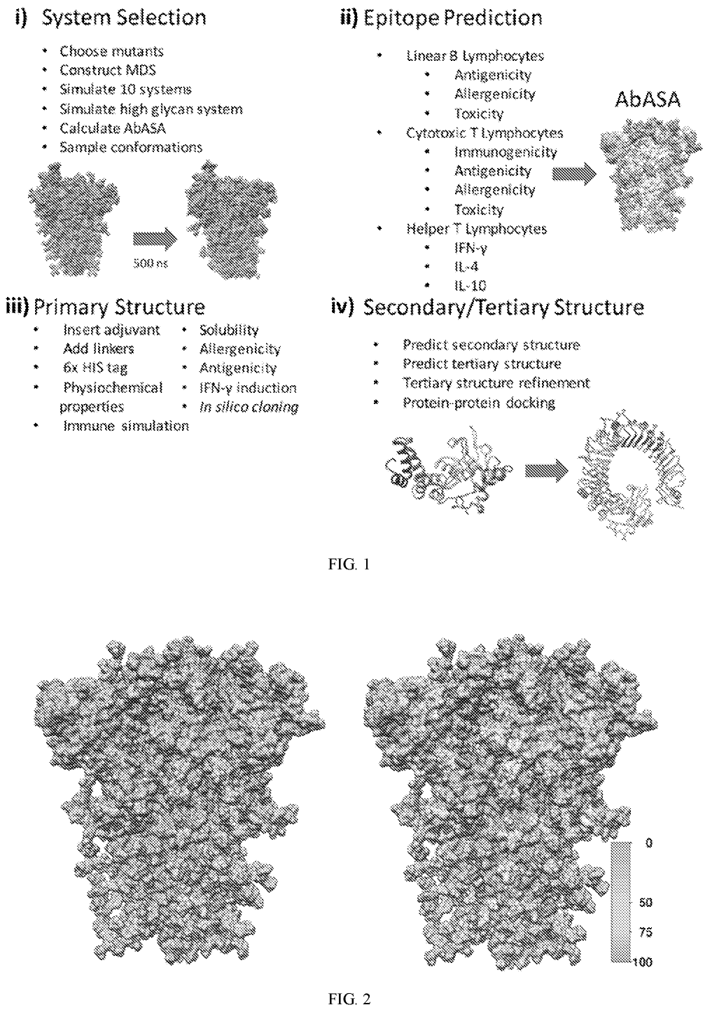

The key to the S protein's ability to ward off an immune response is its considerable glycan shield^{8,9}. The glycosylation of the S glycoprotein creates somewhat of a barrier around the spike, preventing immune molecules from reaching the protein surface. Herein, a multi-epitope vaccine was constructed using molecular dynamics simulations and immunoinformatics techniques while considering the impact of the glycan shield on the ability for a particular epitope to elicit an immune response (FIG. 1). Multiple conformations for the spike glycoprotein were included, both in the wild-type as well as in 9 different mutated states, allowing for a broader reach with respect to B-cell epitope prediction; nearly 75% of the predicted epitopes were exclusive to the predictions from the mutated systems. Additionally, this method was superior to a sequence-based prediction; a preliminary test for linear B-lymphocyte epitopes over the 1,120 amino acid sequence available via crystal structure using BepiPred 2.0¹⁰ yielded only 27 potential epitopes of length 6 or longer.

Each of the simulated systems, including the 9 mutants, wild type, and the high mannose N-glycan substituted wild type system, were assessed for stability along the entire 500 nanosecond simulation using the RMSD of all backbone atoms after least squares fitting to the same using standard GROMACS¹¹ tools (FIG. 9). A total of 5 μs of simulation time was used for linear B lymphocyte prediction. No system was deemed to have any stability issues, so each system was sampled at its initial conformation (after equilibration but before production dynamics simulation) and every 100 nanoseconds of simulation, yielding a total of 6 conformations for each of the 9 mutant and 1 wild type system. The high mannose system was not sampled in this way and was processed separately.

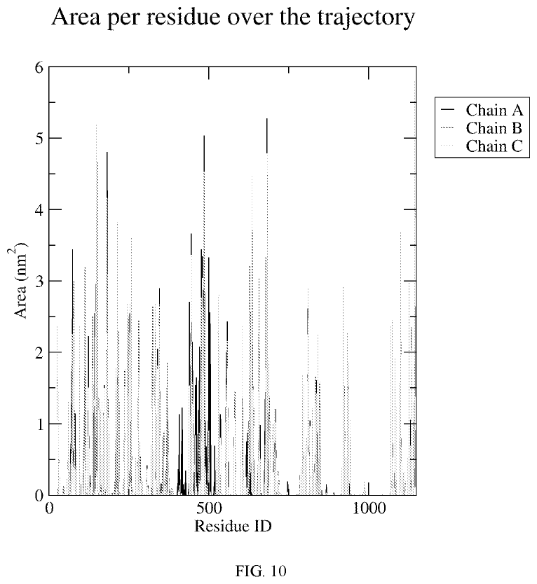

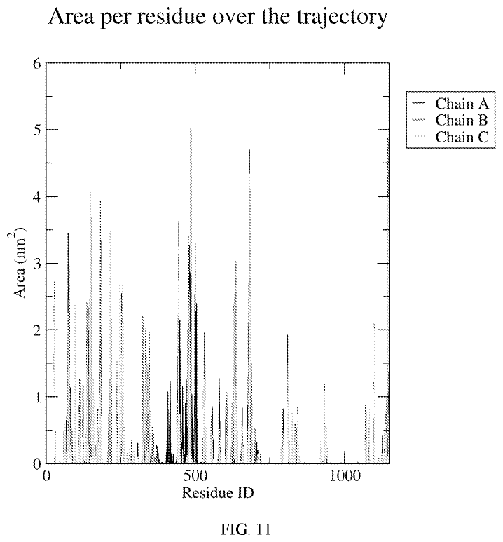

The high mannose system was further assessed for its antibody accessible surface area (AbASA). Using the built in GROMACS tool SASA, with a probe of size 0.72 nm, the surface area was determined for the protein alone (FIGS. 2 and 10) while ignoring the glycosylation, and again while taking the glycosylation into account (FIG. 11). The percent change in the AbASA was determined as the change in the AbASA due to glycosylation (FIG. 2B). This change in accessibility to an immune response was used to dictate which of the predicted epitopes would be included in COVCCF; an epitope was rejected if there was a large change in the AbASA due to the glycosylation, or if there were no residues with greater than 0.25 Å² of accessible surface area. Glycosylation of the spike glycoprotein creates a shield against immune response, reducing the AbASA for many surface residues by over 50%, protecting otherwise targetable epitopes.

### Example 2

### Identification of Epitopes

Antigenic Linear B-cell Epitopes ElliPro¹² was used to predict the linear B-lymphocyte (LBL) epitopes for each of the 6 conformations for each of the nine mutant systems and the wild type. In total, this yielded 3,311 epitopes, of which 428 epitopes were unique (SEQ ID NOs: 1-5; 24-446). Sequences were tested for allergenicity, antigenicity, and toxicity; the sequences which passed these tests were then aligned to the full-length sequence of the viral protein to determine which of the epitopes did not have significant impedance due to the glycosylation. The LBL epitopes included in the final construct are given in Table 1. A total of 5 LBL epitopes were chosen for COVCCF.

Cytotoxic T Lymphocyte Epitopes Using all 10 sequences from the mutated and wild type proteins, a total of 3,844 nonunique CTL epitopes were generated; 260 of these were unique (SEQ ID NOs: 6-11; 447-700). The epitopes which were predicted as immunogenic, antigenic, non-allergenic, and non-toxic were further assessed for their accessibility, yielding 6 total CTL epitopes (Table 1) in the final construct. Epitopes which were either non-antigenic, allergenic, or toxic were not considered; accessibility was determined in the same fashion as for the LBL epitopes.

Helper T Lymphocyte Epitopes As with the CTL prediction, all 10 sequences were submitted to the prediction server, with a total of 3,938 non-unique, and 272 unique, HTL epitopes (SEQ ID NOs: 12-17; 701-966). After predictions for their ability to induce cytokines, and assessment for antibody accessibility, 6 HTL epitopes were included in the final vaccine (Table 1). Epitopes which did not elicit a response from IFN-γ, IL-4, and IL-10 were not considered; accessibility was determined in the same fashion as for the LBL epitopes.

TABLE 1
Predicted epitopes in the final
vaccine construct

| No.* | LBL Epitopes | HTL Epitope | CTL Epitope |
|---|---|---|---|
| 1 | | FEYVSQPFL | |
| | | MDLEGK | |
| | | (SEQ ID | |
| | | NO: 6) | |
| 2 | | FNATRFAS | |
| | | VYAWNRK | |
| | | (SEQ ID | |
| | | NO: 7) | |
| 3 | | | SPRRARSVA |
| | | | (SEQ ID |
| | | | NO: 12) |
| 4 | NSNNLDSKVGGN | | SKVGGNYNY |
| | YNYLY | | (SEQ ID |
| | (SEQ ID | | NO: 13) |
| | NO: 1) | | |
| 5 | IYSKHTPINLVR | | |
| | DLPQGFSALEPLV | | |
| | DLPIG | | |
| | (SEQ ID | | |
| | NO: 2) | | |
| 6 | LPFFSNVTWFHA | LPFFSNVT | HVSGTNGTK |
| | IHVSGTNGTKRF | WFHAIHV | (SEQ ID |
| | DNPVLPF | (SEQ ID | NO: 14) |
| | (SEQ ID | NO: 8) | VTWFHAIHV |
| | NO: 3) | PFFSNVT | (SEQ ID |
| | | WFHAIHVS | NO: 15) |
| | | (SEQ ID | YSKHTPINL |
| | | NO: 9) | (SEQ ID |
| | | | NO: 16) |
| 7 | TPCNGVEGENCY | NGVEGENC | |
| | FPLQSYGFQPT | YFPLQSY | |
| | NG (SEQ ID | (SEQ ID | |
| | NO: 4) | NO: 10) | |
| | | GVEGFNCY | |
| | | FPLQSYG | |
| | | (SEQ ID | |
| | | NO: 11) | |
| 8 | FQTLLALHR | | WTAGA |
| | SYLTPGDSS | | AAYY |
| | SGWTA | | (SEQ ID |
| | GAAAYYV | | NO: 17) |
| | (SEQ ID | | |
| | NO: 5) | | |

*Epitopes are numbered based on their order in the final vaccine construct. Epitopes with a shared sequence also share serial numbers; some LBL epitopes overlapped CTL/HTL epitopes, and some HTL epitopes overlapped CTL epitopes

### Example 3

### Multi-Epitope Polypeptide

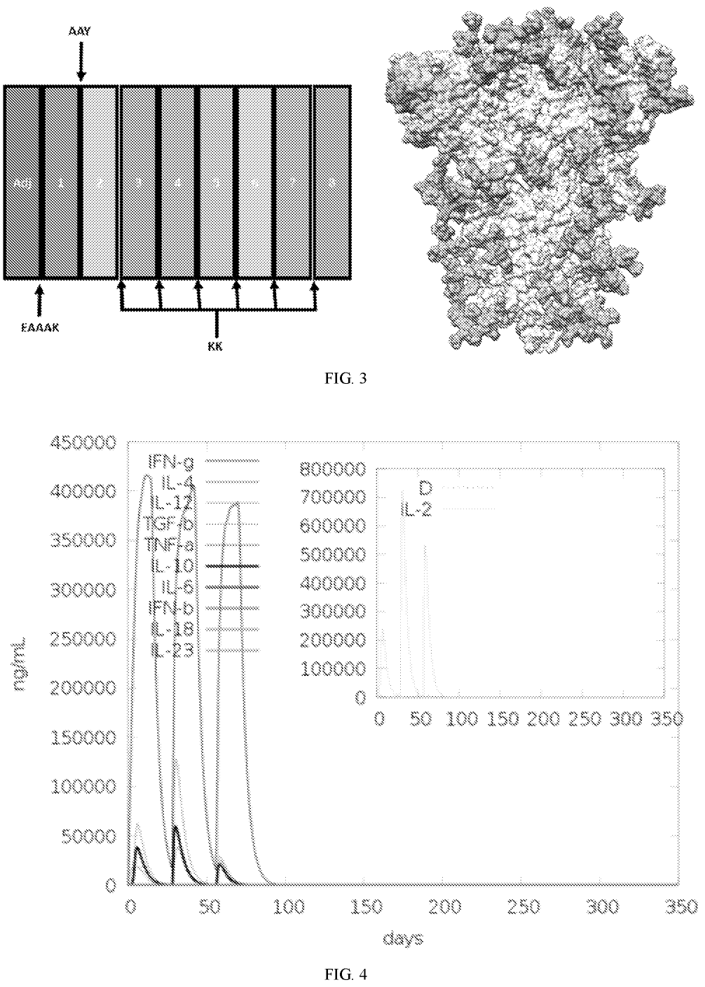

In total, 5 LBL, 6 HTL, and 6 CTL epitopes were selected for COVCCF. Epitopes for which there was overlap were not duplicated in the final construct and were instead merged. The 50S ribosomal L7/L12 protein (UniProt accession ID P9WHE3; SEQ ID NO: 18), a TLR4 agonist, was added as an adjuvant on the N-terminus of the construct and linked to the vaccine peptide using an EAAAK (SEQ ID NO: 19) linker. The GPGPG (SEQ ID NO: 20) linker was chosen between the two HTL epitopes, an AAY linker between the HTL and CTL epitope, and KK linkers between the remaining epitopes. A 6×His tag was added to the C-terminus to aid in purification. COVCCF comprises 331 amino acids and 9 separate linked protein sequences (FIG. 3).

### Example 4

### IFN-γ Inducing Epitope Prediction, Antigenicity and Allergenicity

A total of 323 IFN-γ inducing epitopes were predicted using the scan function of the IFNepitope server¹³. Of these 323 predicted epitopes, 132 were predicted to have positive scores. These results are in line with the simulated levels predicted by C-ImmSim¹⁴ (FIG. 4). The prediction for antigenicity from VaxiJen 2.0¹⁵ indicated it was antigenic under both a bacterial (0.5341) and viral model (0.4709) using the default threshold. COVCCF alone was also determined to be antigenic under both the bacterial (0.6180) and viral (0.5332) models using the default threshold of 0.4. Both the full construct and the vaccine peptide without adjuvant were predicted as nonallergenic using AllerTOP 2.0¹⁶. COVCCF was predicted to be non-allergenic, antigenic, and to elicit IFN-γ induction.

### Example 5

### Physiochemical and Solubility Properties

The physiochemical properties of COVCCF are outlined in Table 2. COVCCF was predicted to have a molecular weight of 35.9 kDa, with a theoretical isoelectric point of 8.75, indicating a slightly basic protein. The half-life was predicted to be 30 hours in mammalian reticulocytes, >20 hours in yeast, and >10 hours in E. coli. The predicted instability index of 27.57 indicated a stable protein (>40 indicates instability), while the aliphatic index of 79.09 indicated thermostability; a larger aliphatic index indicated higher stability. The predicted grand average of hydropathicity was −0.237, which indicated the protein is hydrophilic; this value was calculated as an average over the entire protein of the hydropathicity of each amino acid, where hydrophilic amino acids have a negative value and hydrophobic amino acids have a positive value. The solubility score as determined by CamSol¹⁷ was 0.788 based on the sequence, with a corrected score of 1.994. Altogether, COVCCF was determined to exhibit ideal solubility and physiochemical properties.

TABLE 2
Predicted Physiochemical Properties of COVCCF

| Property | Result | |
|---|---|---|
| Amino acid count | 331 | |
| Molecular Weight | 35906.93 | Da |
| Chemical Formula | C₁₆₃₃H₂₅₂₆N₄₃₂O₄₇₁S₅ | |
| Predicted pI | 8.75 | |
| Estimated half-life | | |
| Mammalian reticulocytes | 30 | hours |
| Yeast Cells | >20 | hours |
| E. coli | >10 | hours |

| | |
|---|---|
| Instability Index | 27.57 |
| Aliphatic Index | 79.89 |
| Grand average of hydropathicity (GRAVY) | −0.237 |
| Solubility | 1.994 |

### Example 6

### Structure Prediction

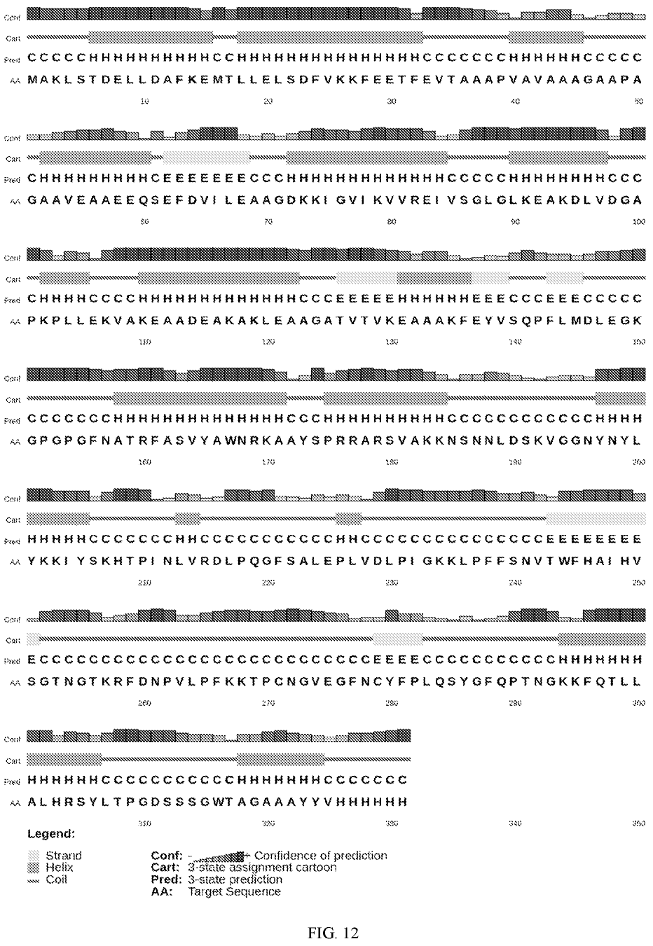

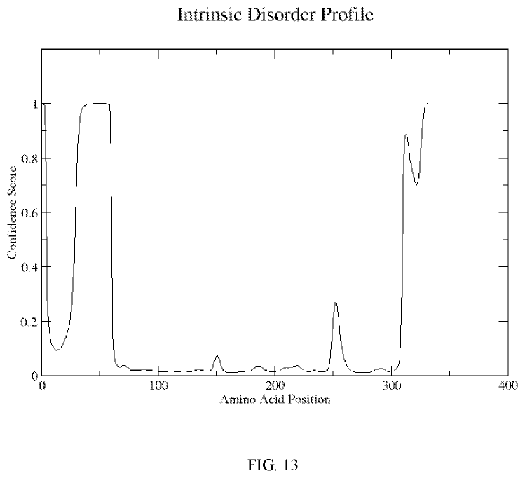

Secondary Structure Prediction The final vaccine sequence was predicted to be 42.6% alpha helix, 9.4% beta sheet, and 48.0% coil by PSIPRED 4.0¹⁸ (FIG. 12), while RaptorX property 1° predicted 37%, 6%, and 56%, respectively. 57% of residues were predicted to be solvent exposed, 24% medium exposed, and 18% buried; a total of 58 residues (17%) were predicted as disordered by RaptorX (FIG. 13). The secondary structure predictions were exported from PSIPRED for use in the tertiary structure modeling.

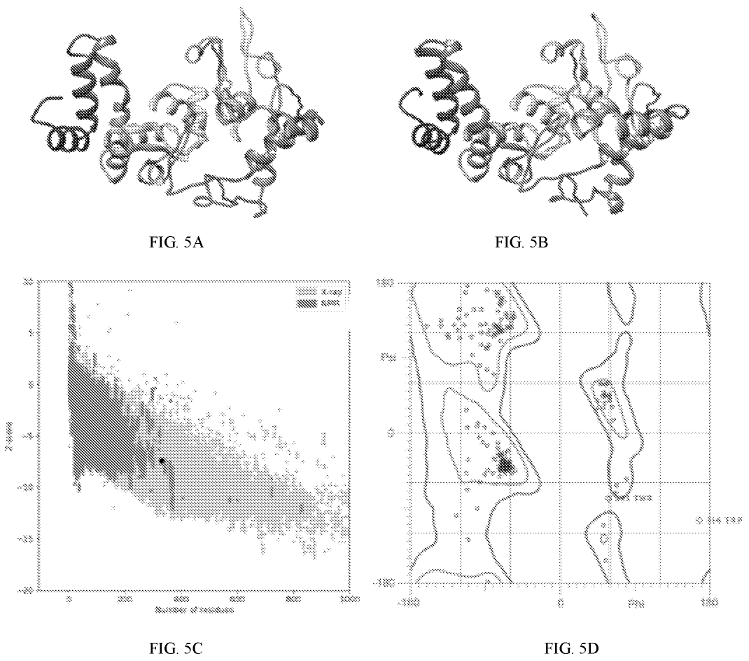

Tertiary Structure Prediction, Refinement, and Validation Five models were predicted using the I-TASSER webserver 2° based on alignments predicted by various threading programs. Z-scores for template alignments ranged from 0.84 to 5.61, with 1rquA, 3qtdA, 1dd3A, 1dd4A, and 2ftc as the top 5 ranking templates. Model 1 (FIG. 5A) was selected for further refinement. A local installation of ModRefiner²¹ was used for the initial refinement of the model in the two-step process outlined in the methods. The model refined using ModRefiner was then submitted to a local installation of GalaxyRefine²², where 10 models were generated for further assessment. The ERRAT server was used to assess the generated model, with model 1 (FIG. 5B) having the highest quality factor of 81.013. Furthermore, ProSA-web²³ was additionally used for validation, indicating a Z-score of −7.41, well within the range of native proteins of comparable size (FIG. The Ramachandran plot indicated 92.7% of residues were in favored regions, 99.4% were in favored or allowed regions, and only two residues were in outlier regions (FIG. 5D). These results indicated the predicted structure was likely close to the actual 3D structure.

Prediction of LBL Epitopes in Final Vaccine Construct A total of 8 discontinuous and 13 linear epitopes were found in COVCCF. The discontinuous epitopes ranged in length from 10 to 46 amino acids, encompassing a total of 213 of the 331 residues in the protein construct. The 13 linear epitopes were nonoverlapping and encompassed 186 residues. Scores ranged from 0.508 to 0.832 for the linear epitopes, and 0.558 to 0.809 for the discontinuous epitopes. This result indicated COVCCF has the ability to induce an immune response not only from the selected epitopes, but from conformational discontinuous epitopes based on its 3D structure.

### Example 7

### Protein-Protein Docking to TLR2 and TLR4

Protein-protein docking was performed using the HADDOCK 2.4 webserver 24 with a data-driven approach. First, CPORT²⁵ was implemented to determine the predicted residues in a protein-protein interaction. Residues from the adjuvant were selected as part of the interaction with both toll-like receptors, since it has been shown as able to induce an immune response²⁶. In the vaccine, residues F32, V34, T35, A36, A38, P39, V42, A43, A45, G46, A48, P49, and A50 were selected to drive the docking, while in the adjuvant alone residues T35, A36, A38, P39, A41, V42, A43, A45, G46, A47, and P49 were chosen. In TLR4, residues 148, D50, NM, L52, P53, F54, S55, D60, P65, H68, G70, S71, Y72, S73, F75, S76, P78, D84, S86, D95, Q99, and S100 were chosen, and residues R63, T65, S85, G87, Y109, Y111 were chosen for TLR2. CPORT predicted many more residues as active, but they were not chosen (Data not shown). Surrounding residues were not entered as input in HADDOCK; instead, the default selection for passive residue selection was used, defining a 6.5 angstrom radius around active residues as the passive region.

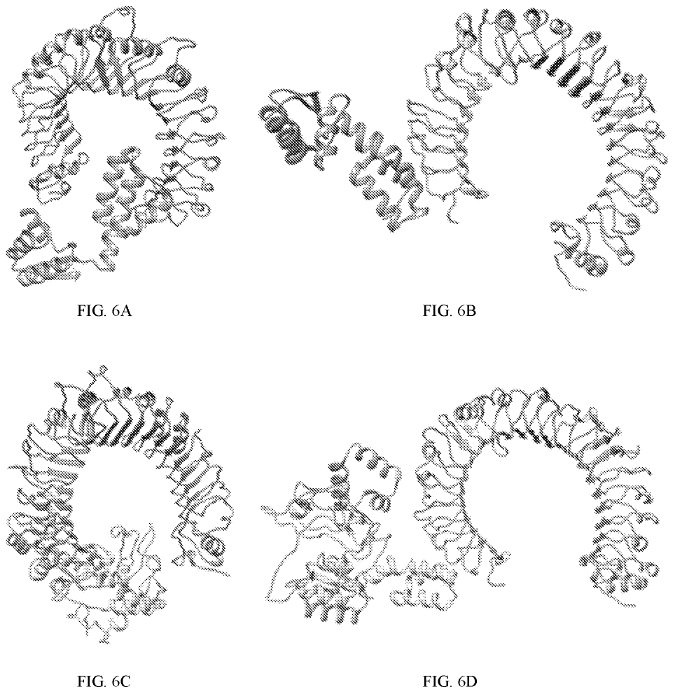

The predicted scores for each of the resulting structures (FIGS. 6A-6D) are outlined in Table 3. The best predicted binding poses for both the TLR2 and TLR4 constructs indicated the vaccine construct induced a conformational change in the adjuvant region which was beneficial to the interaction; the Kd for the vaccine-TLR complexes was comparatively lower in both cases. Without being bound by theory, this was likely due to a predicted increase in the number of interfacial contacts between the complexes. For example, in the TLR4 complexes, four of the six interfacial contact types increased (charged-charged from 3 to 4, charged-polar from 5 to 10, charged-apolar from 5 to 10, and apolar-apolar from 23 to 27), one remained the same (polar-polar at 2) and one decreased (polar-apolar from 13 to 12). Altogether, the docking results indicated that COVCCF is capable of binding to TLR2/TLR4 and inducing an immune response.

TABLE 3
Protein-Protein Docking and Binding Affinity Predictions

| | Cluster | | Best | |
|---|---|---|---|---|
| | Score | | Structure | PRODIGY K_{d} |
| Property | (±)^{‡} | Z-score^{‡} | Score^{†} | Prediction [symbol] |
| Adjuvant - TLR2 | −76.7 (5.0) | −1.4 | −83.422 | 1.3E−07 |
| Vaccine - TLR2 | −84.6 (1.4) | −1.7 | −86.138 | 3.3E−08 |
| Adjuvant - TLR4 | −81.7 (2.9) | −1.4 | −85.447 | 2.6E−07 |
| Vaccine - TLR4 | −84.3 (2.3) | −1.9 | −87.720 | 2.2E−07 |

^{‡}The cluster score and Z-score are the aggregate scores for all proteins within the best cluster.
^{†}Best structure score is for the structure with the lowest HADDOCK score. [symbol]
The PRODIGY prediction is for the predicted best structure by HADDOCK score.

### Example 8

### Codon Optimization

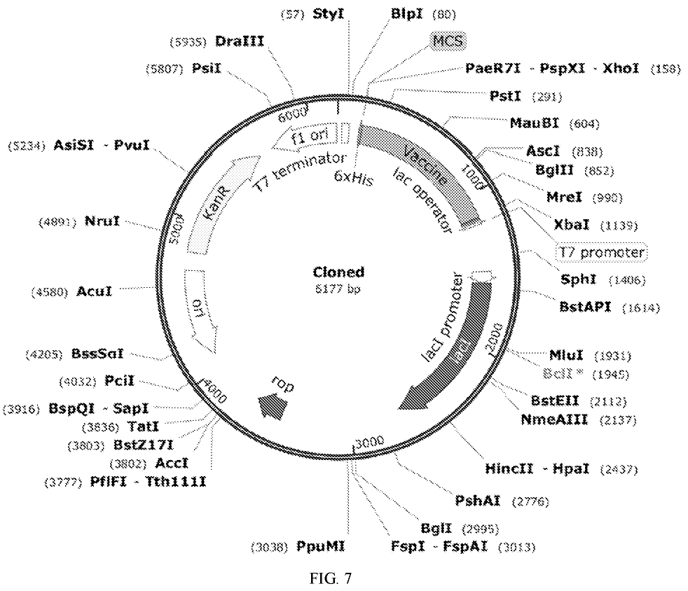

The Java Codon Adaptation Tool²⁷ was used to optimize codon usage of the vaccine construct, to be expressed in E. coli (K12). This optimization would allow for maximal protein expression. A 993 base pair sequence was generated with a Codon Adaptation Index (CAI) value of 0.916, and a GC content of 50.25%, which compared favorably with the 50.73% GC content in the chosen E. coli strain. The sequence of the recombinant plasmid was then inserted in a pET30a (+) vector using SnapGene software (FIG. 7).

### Example 9

### Immune Simulation

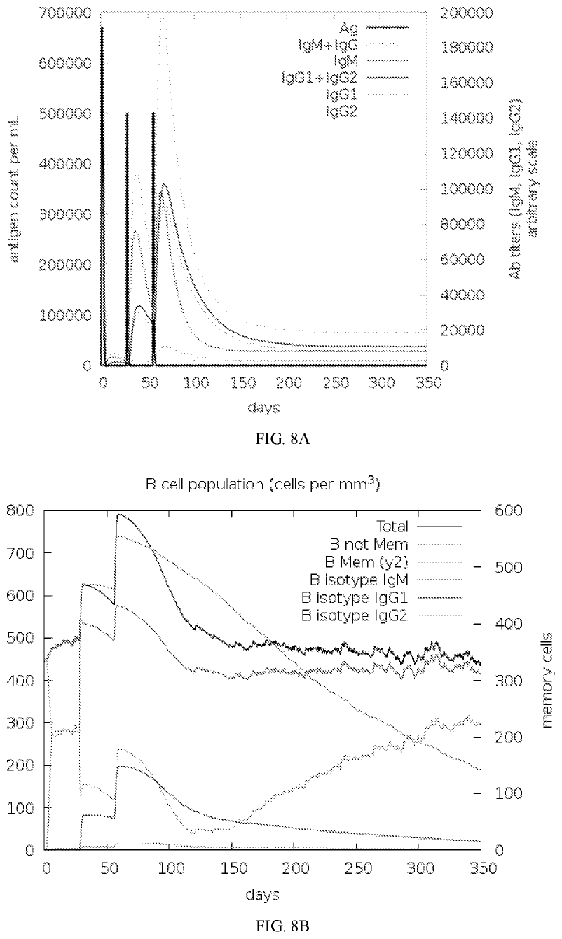

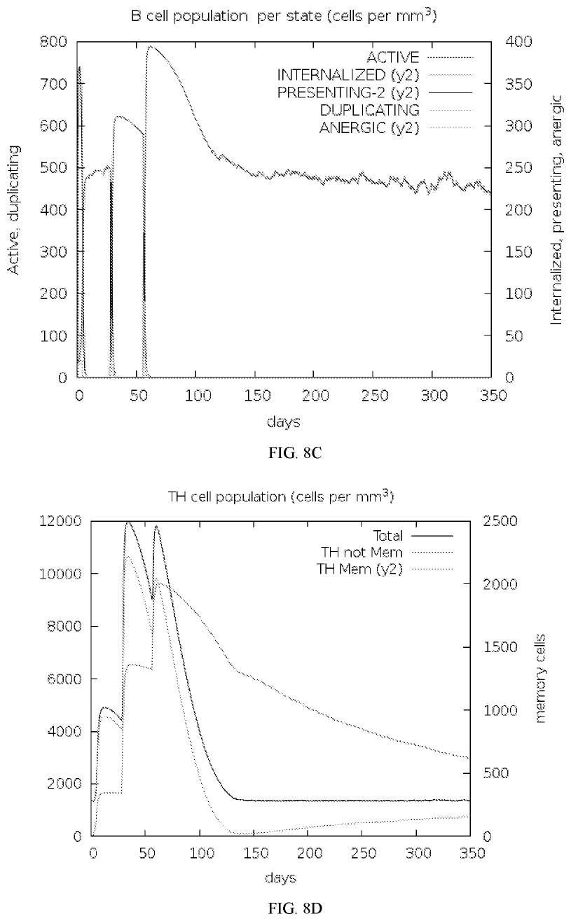

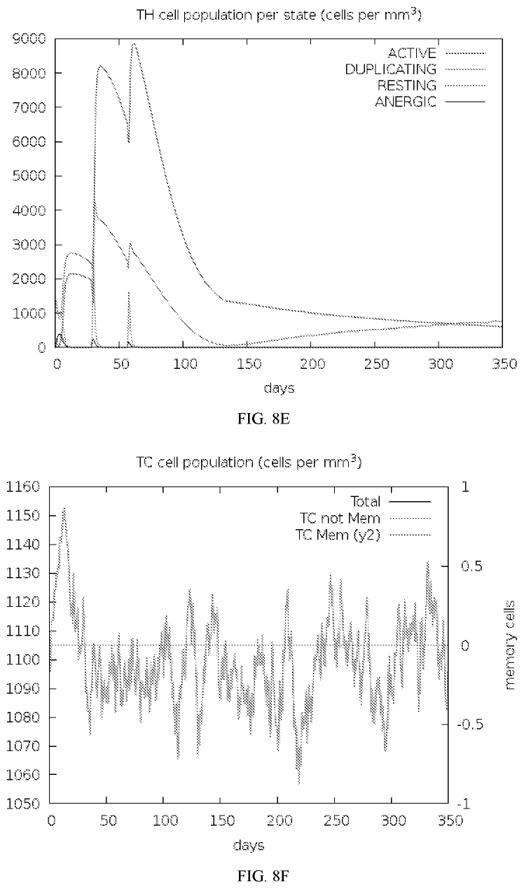

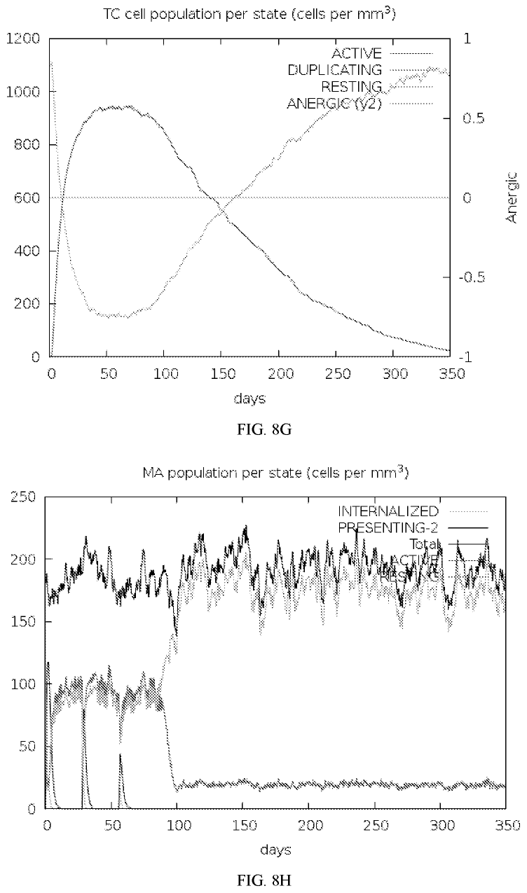

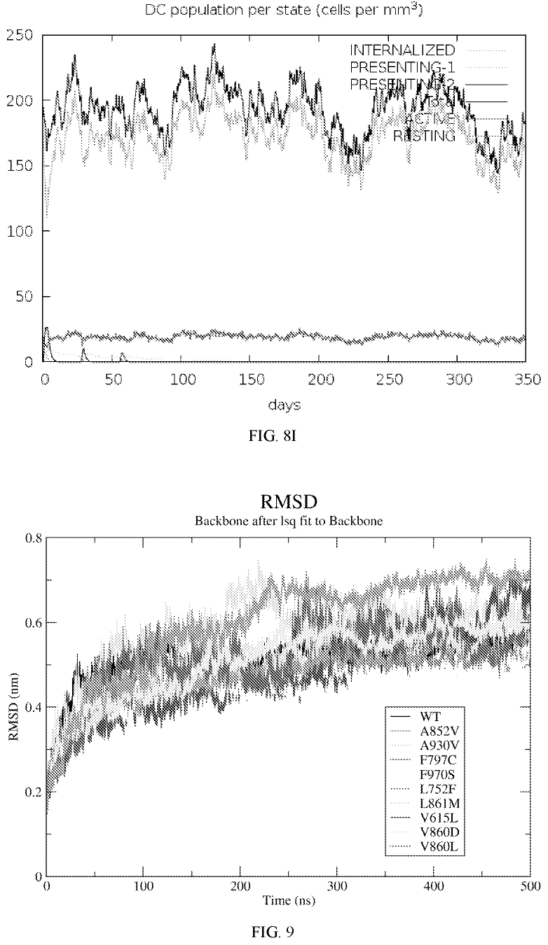

The immune simulations carried out on the C-ImmSim 14 server gave results consistent with an actual immune response, highlighted by the increased secondary response when compared to the primary response. High levels of immunoglobulin activity (IgM, IgG1, IgG2) in the secondary and tertiary response were matched with a corresponding decrease in antigen concentration (Ag, FIG. 8A). B-cell population also increased with each injection (FIGS. 8B-8C), while a corresponding T-helper and cytotoxic T-cell response was evident as well (FIG. 8D-8G). Additionally, macrophage activity was increased, while dendritic cell activity remained consistent during exposure (FIGS. 8H-8I).

As described above, computational techniques, including molecular dynamics simulations and immunoinformatics techniques, were used to design and generate a potential multi-epitope vaccine, coding for multiple LBL, CTL, and HTL epitopes. The full multi-epitope vaccine sequence was predicted to contain 132 IFN-γ positive epitopes; in agreement with the predicted induction of IFN-γ from C-ImmSim, which predicted levels over 400,000 ng/mL for both the primary and secondary doses (FIG. 3) Immune simulation results were consistent with an expected immune response to a vaccine, based on the general increase in the immune response upon secondary and tertiary doses of vaccine. Protein-protein docking comparisons between the full vaccine construct with the adjuvant alone indicated a stronger interaction with both TLR2 and TLR4 in the vaccine than the adjuvant alone, indicating a potential shift in conformation which allows for better binding and potentially a quicker immune response. The inclusion of 9 mutated spike glycoproteins, along with long timescale molecular dynamics simulations, allowed for greater sampling of conformations for the spike glycoprotein; this sampling improved the ability to select LBL epitopes which are immunogenic, antigenic, and are not shrouded by the glycan shield. Thus, the vaccine includes epitopes effective against the SARS-CoV-2 spike glycoprotein in both its unglycosylated and fully glycosylated states

The selected epitopes were effective against the SARS-CoV-2 spike glycoprotein in both its unglycosylated and fully glycosylated states. Some epitopes that may elicit a strong immune response were not included due to the inability of reaching target due to glycan shield. An example of this was predicted LBL epitope from A701 through 1720. It was predicted in all nine mutant systems and the wild type. However, there were no residues in this region which have antibody-accessible surface area in a non-glycosylated protein; glycosylation of residues N709 and N1074 abolished accessibility. As an example, 5704 has 11.4 Å² of AbASA when glycosylation was not accounted for (using a probe size of 0.72 nm) but was reduced to 1.04 Å² of AbASA when glycosylation was taken into account.

As opposed to taking a sequence-based approach to the prediction of LBL epitopes, conformational changes were included which allowed identification of more epitopes. Additionally, multiple mutated systems were included; thereby expanding the predictions. To do this, 500 nanosecond molecular dynamics simulations of 10 different systems, which included 9 mutated systems and the wild-type system. For example, the 9 mutated systems added another 309 unique LBL epitopes not predicted in the 6 conformations used for the wild-type system. In fact, only one out of the five LBL epitopes included in the final vaccine construct was predicted in any of the conformations of the wild-type system.

The multi-epitope polypeptide (COVCCF) consisted of antigenic, nontoxic, non-allergenic, and antibody accessible B-cell and T-cell epitopes; in addition, multiple helper T-cell epitopes, all of which were determined to induce cytokines important to innate immunity, such as IFN-γ, IL4, and IL10, were included. The 35.9 kDa protein was predicted to be soluble upon overexpression in an E. coli host, with a theoretical pI of 8.75, implying its best stability would be in a slightly basic environment. The instability index indicated a protein likely to be stable in a test tube; a protein with an instability index (II) greater than 40 was not predicted to be stable, whereas the II of COVCCF is 27.57. Additionally, the aliphatic index was a positive factor for the increase of thermostability, for which the protein was scored at 79.09. Finally, the negative value for the grand average of hydropathy (GRAVY), -0.237, indicated a hydrophilic protein, allowing it to properly interact with water molecules. The in vivo half-life was predicted using the “Nend rule”; the “N-end rule” relates the half-life of a protein to the identity of the N-terminal residue, which for this protein is a methionine. Outside of an N-terminal valine, this yielded the highest predicted half-life for the vaccine construct, which was a measure of how long it would take for half of the amount of protein in the cell to disappear, based on host.

All publications and patents mentioned in the present application are herein incorporated by reference. Various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

### REFERENCES

1. Tortorici, M. A. & Veesler, D. Structural insights into coronavirus entry. in Advances in Virus Research vol. 105 93-116 (Academic Press Inc., 2019).
2. Hoffmann, M. et al. The novel coronavirus 2019 (2019-nCoV) uses the SARS coronavirus receptor ACE2 and the cellular protease TMPRSS2 for entry into target cells. bioRxiv 2020.01.31.929042 (2020) doi:10.1101/2020.01.31.929042.
3. Ou, X. et al. Characterization of spike glycoprotein of SARS-CoV-2 on virus entry and its immune cross-reactivity with SARS-CoV. Nature Communications 11, 1-12 (2020).
4. Xia, S. et al. Inhibition of SARS-CoV-2 (previously 2019-nCoV) infection by a highly potent pan-coronavirus fusion inhibitor targeting its spike protein that harbors a high capacity to mediate membrane fusion. Cell Research 30, 343-355 (2020).
5. Belouzard, S., Millet, J. K., Licitra, B. N. & Whittaker, G. R. Mechanisms of Coronavirus Cell Entry Mediated by the Viral Spike Protein. Viruses 4, 1011-1033 (2012).
6. Martin, W. R. & Cheng, F. Repurposing of FDA-Approved Toremifene to Treat COVID-19 by Blocking the Spike Glycoprotein and NSP14 of SARS-CoV-2. (2020) doi:10.26434/CHEMRXIV.12431966.V1.
7. Amanat, F. & Krammer, F. SARS-CoV-2 Vaccines: Status Report. Immunity vol. 52 583-589 (2020).
8. Watanabe, Y., Allen, J. D., Wrapp, D., McLellan, J. S. & Crispin, M. Site-specific glycan analysis of the SARS-CoV-2 spike. Science eabb9983 (2020) doi:10.1126/science.abb9983.
9. Watanabe, Y. et al. Vulnerabilities in coronavirus glycan shields despite extensive glycosylation. Nature Communications 11, 2688 (2020).
10. Jespersen, M. C., Peters, B., Nielsen, M. & Marcatili, P. BepiPred-2.0: improving sequence-based B-cell epitope prediction using conformational epitopes. Nucleic Acids Research 45, (2017).
11. Abraham, M. J. et al. GROMACS: High performance molecular simulations through multi-level parallelism from laptops to supercomputers. SoftwareX 1-2, 19-25 (2015).
12. Ponomarenko, J. et al. ElliPro: A new structure-based tool for the prediction of antibody epitopes. BMC Bioinformatics 9, 514 (2008).
13. Dhanda, S. K., Vir, P. & Raghava, G. P. S. Designing of interferon-gamma inducing MHC class-II binders. Biology Direct 8, 30 (2013).
14. Castiglione, F., Mantile, F., de Berardinis, P. & Prisco, A. How the interval between prime and boost injection affects the immune response in a computational model of the immune system. Computational and mathematical methods in medicine 2012, 842329 (2012).
15. Doytchinova, I. A. & Flower, D. R. VaxiJen: A server for prediction of protective antigens, tumour antigens and subunit vaccines. BMC Bioinformatics 8, 4 (2007).
16. Dimitrov, I., Bangov, I., Flower, D. R. & Doytchinova, I. AllerTOP v.2—A server for in silico prediction of allergens. Journal of Molecular Modeling 20, (2014).
17. Sormanni, P, Aprile, F. A. & Vendruscolo, M. The CamSol method of rational design of protein mutants with enhanced solubility. Journal of Molecular Biology 427, 478-490 (2015).
18. Buchan, D. W. A. & Jones, D. T. The PSIPRED Protein Analysis Workbench: 20 years on. Web Server issue Published online 47, (2019).
19. Wang, S., Li, W., Liu, S. & Xu, J. RaptorX-Property: a web server for protein structure property prediction. Nucleic Acids Research 44, (2016).
20. Yang, J. et al. The I-TASSER suite: Protein structure and function prediction. Nature Methods vol. 12 7-8 (2014).
21. Xu, D. & Zhang, Y. Improving the physical realism and structural accuracy of protein models by a two-step atomic-level energy minimization. Biophysical Journal 101, 2525-2534 (2011).
22. Heo, L., Park, H. & Seok, C. GalaxyRefine: protein structure refinement driven by side-chain repacking. doi:10.1093/nar/gkt458.
23. Wiederstein, M. & Sippl, M. J. ProSA-web: interactive web service for the recognition of errors in three-dimensional structures of proteins. Nucleic Acids Research 35, 407-410 (2007).
24. van Zundert, G. C. P. et al. The HADDOCK2.2 Web Server: User-Friendly Integrative Modeling of Biomolecular Complexes. Journal of Molecular Biology 428, 720-725 (2016).
25. de Vries, S. J. & Bonvin, A. M. J. J. CPORT: A Consensus Interface Predictor and Its Performance in Prediction-Driven Docking with HADDOCK. PLoS ONE 6, e17695 (2011).
26. Khatoon, N., Pandey, R. K. & Prajapati, V. K. Exploring Leishmania secretory proteins to design B and T cell multi-epitope subunit vaccine using immunoinformatics approach. Scientific Reports 7, 1-12 (2017).
27. Grote, A. et al. JCat: a novel tool to adapt codon usage of a target gene to its potential expression host. doi:10.1093/nar/gki376.
28. Gori, A., Longhi, R., Peri, C. & Colombo, G. Peptides for immunological purposes: Design, strategies and applications. Amino Acids vol. 45 257-268 (2013).
29. Kim, Y. it et al. Infection and Rapid Transmission of SARS-CoV-2 in Ferrets. Cell Host and Microbe 27, 704-709.e2 (2020).
30. Park, S. J. et al. Antiviral efficacies of FDA-approved drugs against SARS-COV-2 infection in ferrets. mBio 11, (2020).
31. Daniloski, Z., Guo, X. & Sanjana, N. E. The D614G mutation in SARS-CoV-2 Spike increases transduction of multiple human cell types. bioRxiv 2020.06.14.151357 (2020) doi:10.1101/2020.06.14.151357.
32. Koyama, T., Weeraratne, D., Snowdon, J. L. & Parida, L. Emergence of Drift Variants That May Affect COVID-19 Vaccine Development and Antibody Treatment. Pathogens 9, 324 (2020).
33. Lee, J. et al. CHARMM-GUI Input Generator for NAMD, GROMACS, AMBER, OpenMM, and CHARMM/OpenMM Simulations Using the CHARMM36 Additive Force Field. Journal of Chemical Theory and Computation 12, 405-413 (2016).
34. Jo, S., Kim, T., Iyer, V. G. & Im, W. CHARMM-GUI: A web-based graphical user interface for CHARMM. Journal of Computational Chemistry 29, 1859-1865 (2008).
35. Wrapp, D. et al. Cryo-EM structure of the 2019-nCoV spike in the prefusion conformation. Science (New York, N.Y) 367, 1260-1263 (2020).
36. Best, R. B. et al. Optimization of the Additive CHARMM All-Atom Protein Force Field Targeting Improved Sampling of the Backbone ϕ, φ and Side-Chain χ₁ and χ₂ Dihedral Angles. Journal of Chemical Theory and Computation 8, 3257-3273 (2012).
37. Hess, B., Bekker, H., Berendsen, H. J. C. & Fraaije, J. G. E. M. LINCS: A linear constraint solver for molecular simulations. Journal of Computational Chemistry 18, 1463-1472 (1997).
38. Braga, C. & Travis, K. P. A configurational temperature Nose-Hoover thermostat. The Journal of Chemical Physics 123, 134101 (2005).
39. Parrinello, M. & Rahman, A. Polymorphic transitions in single crystals: A new molecular dynamics method. Journal of Applied Physics 52, 7182-7190 (1981).
40. Verlet, L. Computer “Experiments” on Classical Fluids. I. Thermodynamical Properties of Lennard-Jones Molecules. Physical Review 159, 98-103 (1967).
41. Darden, T., York, D. & Pedersen, L. Particle mesh Ewald: An N log(N) method for Ewald sums in large systems. The Journal of Chemical Physics 98, 10089-10092 (1993).
42. Grant, O. C., Montgomery, D., Ito, K. & Woods, R. J. Analysis of the SARS-CoV-2 spike protein glycan shield: implications for immune recognition. bioRxiv 2020.04.07.030445 (2020) doi:10.1101/2020.04.07.030445.
43. Nain, Z. et al. Proteome-wide screening for designing a multi-epitope vaccine against emerging pathogen Elizabethkingia anophelis using immunoinformatic approaches. Journal of Biomolecular Structure and Dynamics (2019) doi:10.1080/07391102.2019.1692072.
44. Larsen, M. v. et al. Large-scale validation of methods for cytotoxic T-lymphocyte epitope prediction. BMC Bioinformatics 8, (2007).
45. Calis, J. J. A. et al. Properties of MHC Class I Presented Peptides That Enhance Immunogenicity. PLoS Computational Biology 9, (2013).
46. Nielsen, M., Lundegaard, C. & Lund, 0. Prediction of MHC class II binding affinity using SMM-align, a novel stabilization matrix alignment method. BMC Bioinformatics 8, 238 (2007).
47. Wang, P. et al. Peptide binding predictions for HLA DR, DP and DQ molecules. BMC Bioinformatics 11, (2010).
48. Nielsen, M. & Lund, 0. NN-align. An artificial neural network-based alignment algorithm for MHC class II peptide binding prediction. BMC Bioinformatics 10, 296 (2009).
49. Sidney, J. et al. Quantitative peptide binding motifs for 19 human and mouse MHC class i molecules derived using positional scanning combinatorial peptide libraries. Immunome Research 4, (2008).
50. Sturniolo, T. et al. Generation of tissue-specific and promiscuous HLA ligand databases using DNA microarrays and virtual HLA class II matrices. Nature Biotechnology 17, 555-561 (1999).
51. Andreatta, M. et al. Accurate pan-specific prediction of peptide-MHC class II binding affinity with improved binding core identification. Immunogenetics 67, 641-650 (2015).
52. Wold, S., Jonsson, J., Sjorstrom, M., Sandberg, M. & Rannar, S. DNA and peptide sequences and chemical processes multivariately modelled by principal component analysis and partial least-squares projections to latent structures. Analytica Chimica Acta 277, 239-253 (1993).
53. Gupta, S. et al. In Silico Approach for Predicting Toxicity of Peptides and Proteins. PLoS ONE 8, e73957 (2013).
54. Luckheeram, R. V., Zhou, R., Verma, A. D. & Xia, B. CD4+ T Cells: Differentiation and Functions. Clinical and Developmental Immunology 2012, 12 (2012).
55. Dhanda, S. K., Gupta, S., Vir, P. & Raghava, G. P. S. Prediction of IL4 Inducing Peptides. Clinical and Developmental Immunology 2013, 263952 (2013).
56. Nagpal, G. et al. Computer-aided designing of immunosuppressive peptides based on IL-10 inducing potential. Scientific Reports 7, (2017).
57. Gu, Y. et al. Vaccination with a paramyosin-based multi-epitope vaccine elicits significant protective immunity against Trichinella spiralis infection in mice. Frontiers in Microbiology 8, (2017).
58. Gasteiger, E. et al. Protein Identification and Analysis Tools on the ExPASy Server. http://www.expasy.org/tools/.
59. Sormanni, P., Amery, L., Ekizoglou, S., Vendruscolo, M. & Popovic, B. Rapid and accurate in silico solubility screening of a monoclonal antibody library. Scientific Reports 7, 1-9 (2017).
60. Zheng, W. et al. LOMETS2: improved meta-threading server for fold-recognition and structure-based function annotation for distant-homology proteins. Nucleic Acids Research 47, 429-436 (2019).
61. Colovos, C. & Yeates, T. O. Verification of protein structures: Patterns of nonbonded atomic interactions. Protein Science 2, 1511-1519 (1993).
62. Chen, V. B. et al. MolProbity: All-atom structure validation for macromolecular crystallography. Acta Crystallographica Section D: Biological Crystallography 66, 12-21 (2010).
63. Barlow, D. J., Edwards, M. S. & Thornton, J. M. Continuous and discontinuous protein antigenic determinants Nature 322, 747-748 (1986).
64. van Regenmortel, M. H. V. Mapping epitope structure and activity: From one-dimensional prediction to four-dimensional description of antigenic specificity. Methods: A Companion to Methods in Enzymology 9, 465-472 (1996).
65. Xue, L. C., Rodrigues, J. P., Kastritis, P. L., Bonvin, A. M. & Vangone, A. PRODIGY: a web server for predicting the binding affinity of protein-protein complexes. Bioinformatics (Oxford, England) 32, 3676-3678 (2016).
66. Rapin, N., Lund, O., Bernaschi, M. & Castiglione, F. Computational immunology meets bioinformatics: The use of prediction tools for molecular binding in the simulation of the immune system. PLoS ONE 5, e9862 (2010).

What is claimed is:

1. A composition comprising one or more polypeptides comprising a plurality of epitopes from the spike glycoprotein of SARS-CoV-2,
wherein the plurality of epitopes comprises at least one of each of: a linear B lymphocyte (LBL) epitope; a cytotoxic T lymphocyte (CTL) epitope; and a helper T lymphocyte (HTL) epitope.

2. The composition of claim 1, wherein at least a portion of the plurality of epitopes are non-contiguous epitopes from the spike glycoprotein of SARS-CoV-2.

3. The composition of claim 1 or claim 2, wherein the one or more polypeptides comprise between one and five LBL epitopes.

4. The composition of any of claims 1-3, wherein the one or more polypeptides comprise five LBL epitopes.

5. The composition of any of claims 1-4, wherein each LBL epitope comprises an amino acid sequence selected from the group consisting of: SEQ ID NOs: 1-5 and 24-446.

6. The composition of any of claims 1-5, wherein each LBL epitope comprises an amino acid sequence selected from the group consisting of: SEQ ID NOs: 1-5.

7. The composition of any of claims 1-6, wherein the one or more polypeptides comprise between one and six HTL epitopes.

8. The composition of any of claims 1-7, wherein the one or more polypeptides comprise six HTL epitopes.

9. The composition of any of claims 1-8, wherein each HTL epitope comprises an amino acid sequence selected from the list consisting of: SEQ ID NOs: 6-11 and 447-700.

10. The composition of any of claims 1-9, wherein each HTL epitope comprises an amino acid sequence selected from the list consisting of: SEQ ID NOs: 6-11.

11. The composition of any of claims 1-10, wherein the one or more polypeptides comprise between one and six CTL epitopes.

12. The composition of any of claims 1-11, wherein the one or more polypeptides comprise six CTL epitopes.

13. The composition of any of claims 1-12, wherein each CTL epitope comprises an amino acid sequence selected from the list consisting of: SEQ ID NOs: 12-17 and 701-966.

14. The composition of any of claims 1-13, wherein each CTL epitope comprises an amino acid sequence selected from the list consisting of: SEQ ID NOs: 12-17.

15. The composition of any of claims 1-14, wherein the plurality of epitopes comprises fully overlapping, partially overlapping, and non-overlapping epitopes.

16. The composition of claim 15, wherein the one or more polypeptides further comprise a linker between non-overlapping epitopes.

17. The composition of any of claims 1-16, wherein the linker comprises an amino acid sequence of AAY, KK, or GPGPG (SEQ ID NO: 20).

18. The composition of any of claims 1-16, further comprising an adjuvant.

19. The composition of claim 18, wherein the adjuvant comprises a peptide adjuvant.

20. The composition of any of claims 18-19, wherein the adjuvant comprises 50S ribosomal L7/L12 protein.

21. The composition of any of claims 18-20, wherein the adjuvant is conjugated to the one or more polypeptides N-terminus with a linker.

22. The composition of claim 21, wherein the linker comprises an amino acid sequence of SEQ ID NO: 19.

23. The composition of any of claims 1-22, wherein the composition comprises one polypeptide comprising the plurality of non-contiguous epitopes.

24. The composition of claim 23, wherein the polypeptide comprises an amino acid sequence with at least 70% similarity to SEQ ID NO: 21, SEQ ID NO: 22 or SEQ ID NO: 23.

25. The composition of any of claims 1-24, further comprising at least one carrier.

26. The composition of claim 25, wherein said carrier comprises a physiological tolerable buffer.

27. A method for reducing or preventing a viral infection in a subject in need thereof, comprising administering to the subject an effective amount of the composition of any of claims 1-26.

28. The method of claim 27, wherein the viral infection comprises a coronavirus infection.

29. The method of claim 28, wherein the coronavirus is severe acute respiratory syndrome coronavirus-2 (SARS-CoV-2).

30. The method of any of claims 27-29, wherein the administering comprises an initial immunization and at least one subsequent immunization.

31. A method of inducing an immune response in a subject comprising administration of the composition of any of claims 1-26.

32. The method of any of claims 28-31, wherein the subject is human.

33. Use of the composition of any of claims 1-26 in the manufacture of a medicament for the treatment or prevention a viral infection.

34. The use of claim 33, wherein the viral infection comprises a coronavirus infection

35. The use of claim 34, wherein the coronavirus is severe acute respiratory syndrome coronavirus-2 (SARS-CoV-2).

36. The use of any of claims 33-35, wherein the administering comprises an initial immunization and at least one subsequent immunization.

37. The use of any of claims 33-36, wherein the subject is human.

38. A nucleic acid encoding a polypeptide comprising a plurality of non-contiguous epitopes from the spike glycoprotein of SARS-CoV-2,
wherein the plurality of epitopes comprises at least one of each of: a linear B lymphocyte (LBL) epitope; a cytotoxic T lymphocyte (CTL) epitope; and a helper T lymphocyte (HTL) epitope.

39. An expression vector comprising the nucleic acid of claim 38 in combination with a promoter.

40. A system comprising:
the composition of any one or claims 1-26; and
a delivery device or a container.

41. The system of claim 40, wherein the delivery device comprises a syringe.

42. The system of claim 40 or 41, wherein the composition is pre-loaded in the delivery device.

43. The system of claim 40, wherein said container comprises a syringe vial.

44. The system of claim 43, wherein said composition is in the syringe vial.

45. The system of any of claims 40-44, further comprising a packaging component.

46. The system of claim 45, wherein the packaging component contains the container, and the composition is inside the container.
